(19) United States
(12) Patent Application Publication
(10) Pub. No.: US 2023/0097123 A1
(43) Pub. Date: Mar. 30, 2023

(54) METHODS AND KITS FOR IDENTIFYING, DIAGNOSING AND TREATING PROSTATE CARCINOMAS

(71) Applicant: 10x Genomics, Inc., Pleasanton, CA (US)

(72) Inventors: Valeria GIANGARRA, Rome (IT); James Michael CHELL, Stockholm (SE); Stephen R. WILLIAMS, Crozet, VA (US); Mesruh TURKEKAL, Taby (SE); Paulius MIELINIS, Stockholm (SE); Caroline Julie GALLANT, Uppsala (SE); Sarah E. B. TAYLOR, Dublin, CA (US); Aleksandra JUREK, Stockholm (SE)

(73) Assignee: 10x Genomics, Inc., Pleasanton, CA (US)

(21) Appl. No.: 17/956,596

(22) Filed: Sep. 29, 2022

Related U.S. Application Data
(60) Provisional application No. 63/261,922, filed on Sep. 30, 2021.
(60) Provisional application No. 63/306,939, filed on Feb. 4, 2022.

(51) Int. Cl. C12Q 1/6886

(52) U.S. Cl. CPC C12Q 1/6886; C12Q 2600/106; C12Q 2600/158; C12Q 2600/118

(57) ABSTRACT

The present disclosure generally relates to methods, systems and compositions for determining the presence or absence of prostate cancer in a subject, treating prostate cancer in a subject, and monitoring the efficacy of a treatment regimen for prostate cancer in a subject. The methods of the present disclosure comprise determining the expression levels and spatial locations of two or more analytes in a sample from a subject and correlating the expression levels and spatial locations to a prostate sample from a subject, thereby determining the presence or absence of prostate cancer, determining a treatment regimen for prostate cancer, or monitoring the efficacy of an existing treatment regimen in a subject undergoing treatment for prostate cancer.

### CROSS-REFERENCE TO RELATED PATENT APPLICATIONS

This application claims priority to and the benefit of U.S Provisional Patent Application No. 63/261,922, filed Sep. 30, 2021, and U.S. Provisional Patent Application No. 63/306,939, filed Feb. 4, 2022, the disclosures of which are incorporated herein by reference in their entirety.

### FIELD

The present disclosure generally relates to methods of diagnosing the presence or absence of prostate cancer in a subject. The present disclosure further generally relates to methods for treating prostate cancer in a subject having prostate cancer and also to methods of preventing and/or reducing the incidence of prostate cancer in a subject having an increased likelihood of developing prostate cancer. In some aspects, the methods of the present disclosure comprise determining the expression level of one or more analytes in a sample from a subject and correlating the expression level to a stage of prostate cancer or a likelihood of developing prostate cancer.

### BACKGROUND

Prostate cancer is the second most common cancer diagnosis in males in the world and the fourth most common cancer overall according to the World Cancer Research Fund/American Institute for Cancer Research. In 2021 alone the American Cancer Society estimates that around 250,000 American men will be diagnosed with a form of prostate cancer, and around 34,000 American men will succumb to the disease. For further perspective, one in eight men in the United States will be diagnosed with a form of prostate cancer in his lifetime, and the disease remains the second leading cause of death in American males behind lung cancer as about 1 in 41 men will die of prostate cancer post-diagnosis, according to the American Cancer Society.

Early detection of prostate cancer is critical for survival as early detection can provide patients with multiple treatment options that may not be available for more advanced disease stages. However, current means of early detection, such as PSA tests and digital rectal exams (DREs), are often subject to false positive or false negative results. As such, clinical challenges have arisen due to the lack of a standalone test to assess for prostate cancer or prostate cancer risk. In fact, the aforementioned methods are so unreliable that an actual diagnosis for the presence of prostate cancer is made with a biopsy of the prostate.

A prostate biopsy generally involves multiple core needle biopsy samples being obtained, processed, and stained prior to analysis by a pathologist. A pathologist then reviews one or more slices from the multiple biopsies via histological and immunohistochemical (IHC) methods, and categorizes the biopsy samples by determining the presence, absence, amount, and/or location of tumor cells in the biopsied tissues. The pathologist also grades the biopsy tissues by applying a “Gleason score” as an indicator of the severity of any cancer that is present in the sample. Gleason scores of 5 or lower generally indicate a non-cancerous sample. The lowest Gleason score indicating a cancerous sample is 6, which indicates a low-grade cancer. A Gleason score of 7 is a medium-grade cancer, and a Gleason score of 8, 9, or 10 is a high-grade cancer. Based on the summation of these subjective analyses, i.e., the pathologist's annotations of the stained sample and the Gleason score assigned by the pathologist to the sample, the pathologist then diagnoses the stage (I-IV) of any cancerous tissue present in the sample. In spite of this pathological analysis, clinical false negatives and false positives still occur, further hindering the identification and treatment of prostate cancer.

As mentioned above, the tests used to determine the risk or stage of prostate cancer are subjective and often produce false positive or false negative results, thereby complicating accurate diagnosis and treatment of any prostate cancer that may be present in a subject, or accurate risk assessment of the likelihood of a subject developing prostate cancer. As such, there remains a need in the field for methods to accurately assess the stage of prostate cancer in a subject, as well as methods and kits for treating prostate cancer in a subject, or preventing, and/or reducing the incidence of prostate cancer in a subject having an increased likelihood of developing prostate cancer.

### SUMMARY OF THE INVENTION

Methods for accurately detecting, diagnosing, and monitoring for prostate disease are subjective and suffer from false positive and false negative results. The present disclosure provides methods for determining the expression levels correlated with the presence and/or absence of prostate cancer. Such methods provide for a diagnostician to more accurately determine the presence or absence of prostate disease that is specific to a subject, thereby advancing personalized medicine initiatives for prostate disease.

In some aspects, a method of treating prostate cancer in a subject comprises providing or having provided a biological sample from the subject and contacting or having contacted the biological sample with a spatial gene expression array, determining or having determined the expression level of two or more analytes of one or more of the groups of (i) through (iv); (i) S100B, TGM4, SMOC1, LTF, ANPEP, SELE, PSCA, MT1G, ACTG2, WFDC2, SOCS3, PCP4, SYNM, GADD45B, TNC, GAS1, CNN1, ZNF185, DES, HSPB6, MSMB, MYLK, IGHG1, FLNC, ADAMTS4, MYL9, ADAM33, KRT5, PGM5, IGHV3-35, NEFH, JPH2, KRT15, TRIM29, SRD5A2, HSPB8, C11orf96, SPEG, ALDH2, FLNA, OLFM4, IGKV1D-12, TAGLN, TPM2, CRYAB, SMTN, KCNMB1, PMP22, ACTA2, and SYNPO2, or a fragment thereof; (ii) ADGRF1, OR51C1P, KCNC2, PLA2G7, SPON2, CAMK2N1, KCNN2, OR51E2, AGTR1, GRIN3A, KHDRBS3, KCNH8, ATP8A2, TMEM26, LUZP2, HPN, PLA2G2A, COL2A1, UNC80, DNASE2B, HOXC6, SPOCK1, AKR7A3, LRRN1, GOLM1, GJB1, FGFRL1, GHR, ACSM3, MCCC2, TRGV9, ZNF385B, CYP39A1, SEC11C, H2AFJ, MYO6, GPR160, BMPR1B, MBOAT2, GALNT3, CPNE4, ACER3, PLEKHH1, C9orf152, and PAQR6, or a fragment thereof; (iii) OR51E1, FGFR3, SPON2, KCNG3, OR51C1P, ARG2, DEGS1, OR51E2, TRGJP2, CPNE4, GOLM1, RDH11, ACSM1, PLEKHH1, SLC30A4, TRGC1, GDF11, KHDRBS3, SPOCK1, GDF15, FGFRL1, TRGV9, LRRN1, PLA2G7, TMSB15A, KCNN2, MARC1, PTPRT, C5orf30, COLEC12, HPN, MYO6, ADGRF1, UBE2E3, HGD, NSD2, KIF21A, HIST1H2AC, LETM1, COL9A2, BEND4, GJB1, ARFGEF3, PRAC1, F3, and SAMD5, or a fragment thereof; (iv) IGHA1, JCHAIN, CD79A, IGHJ6, IGKC, SAA2, CCL19, IGLC1, SAA1, IGHG2, POU2AF1, DERL3, S100A9, IGHG1, LTB, C3, MZB1, IGKV4-1, RARRES1, LTF, IGHG3, IGHJ2, IGLV3-1, LYZ, LSP1, OLFM4, FCGBP, CFB, PDZK1IP1, KRT7, CORO1A, IL7R, PRDM1, CYBA, IGHM, LCN2, SERPINF1, HCLS1, CD53, PTPRC, PTGDS, NNMT, IGFBP4, WFDC2, CP, ITGB2, C1R, CLU, KRT15, and FHL2, or a fragment thereof; in the biological sample from the subject, correlating or having correlated the determined expression level to a stage of prostate cancer, and proceeding with one or more of (i) administering a prostate cancer treatment to the subject based on the determined expression level of the one or more analytes, (ii) adjusting a dosage of a current prostate cancer treatment for the subject based on the determined expression level of the one or more analytes, or (iii) adjusting a prostate cancer treatment for the subject based on the determined expression level of the one or more analytes.

In other aspects, a method of preventing or reducing the incidence of prostate cancer in a subject having an increased likelihood of developing prostate cancer, comprises providing or having provided a biological sample from the subject and contacting or having contacted the biological sample with a spatial gene expression array, determining or having determined the expression level of two or more analytes of one or more of the pre-defined groups (i) through (iv) in the biological sample from the subject, correlating or having correlated the determined expression level to the likelihood of developing prostate cancer, and proceeding with one or more of (i) administering a prophylactic prostate cancer treatment to the subject based on the determined expression level of the one or more analytes, (ii) adjusting a prophylactic prostate cancer treatment dosage of a current prostate cancer prophylactic treatment for the subject based on the determined expression level of the one or more analytes, or (iii) adjusting a prophylactic prostate cancer treatment for the subject based on the determined expression level of the one or more analytes.

In other aspects, a method of treating prostate cancer in a subject comprises providing or having provided a biological samples from the subject and contacting or having contacted the biological sample with a spatial gene expression array, determining or having determined the expression level of two or more analytes of one or more of the pre-defined groups (i) through (iv) in the biological sample from the subject, correlating or having correlated the determined expression level to a stage of prostate cancer, identifying or having identified one or more luminal cells or an analyte associated with a luminal cell in one or more spatial locations in the biological sample, determining or having determined the degree of luminal cell infiltration in the cancerous region of the biological sample obtained from the subject and proceeding with one or more of (d) administering a prostate cancer treatment to the subject, adjusting a dosage of a current prostate cancer treatment for the subject or adjusting a prostate cancer treatment for a subject based on the determined expression level of the one or more analytes and the degree of luminal cell infiltration. Disclosed herein is additionally a method of preventing or reducing the incidence of prostate cancer in a subject having an increased likelihood of developing prostate cancer by analyte expression level and luminal cell infiltration as previously described.

In some aspects, the methods described herein are related to treating prostate cancer in a subject or preventing or reducing the incidence of prostate cancer in a subject having an increased likelihood of developing prostate cancer, comprising providing or having provided a biological samples from the subject and contacting or having contacted the biological sample with a spatial gene expression array, determining or having determined the expression level of two or more analytes of one or more of the pre-defined groups (i) through (iv) in the biological sample from the subject, correlating or having correlated the determined expression level to a stage of prostate cancer, determining or having determined the degree of immune cell infiltration in the cancerous region of the biological sample obtained from the subject and proceeding with one or more of (d) administering a prostate cancer treatment to the subject, adjusting a dosage of a current prostate cancer treatment for the subject or adjusting a prostate cancer treatment for a subject based on the determined expression level of the one or more analytes and the degree of immune cell infiltration. Disclosed herein is additionally a method of preventing or reducing the incidence of prostate cancer in a subject having an increased likelihood of developing prostate cancer by analyte expression level and immune cell infiltration as previously described.

In some aspects, methods herein can be used to determine the presence or absence of prostate cancer in a subject comprising contacting a biological sample with a spatial gene expression array and determining the expression level of two or more analytes of one or more of the groups (i) through (iv) in the biological sample from the subject, and correlating the determined expression level of the one or more analytes with the presence or absence of prostate cancer in the sample from the subject. In some aspects, the expression level and/or spatial location of one or more luminal cell biomarkers such as CD24, KRT8 and KRT18, or combinations thereof can be used to determine the presence or absence of prostate cancer in a subject. In some aspects, the expression level and/or spatial location of one or more immune cell biomarkers such as CD3D, CD3E, CD4, CD8A, CD247, CD79A, CD79B, IGHA1, IGHG1, JCHAIN, IGKC, IGLC1, or combinations thereof can be used to determine the presence or absence of prostate cancer in a subject. In some aspects, immune cells that can be monitored for gene expression analytes include B cells, non-plasma B cells, plasma B cells, tumor infiltrating B cells, T cells, regulatory T cells, cytotoxic T cells, monocytes, macrophages, natural killer cells, or combinations thereof. For example, analytes for expression level analysis for use in methods described herein include, but are not limited to, CD3+, CD4+, CD8+T cell analytes, a regulatory T cell comprising two or more of CD4, FOXP3, IL17RB, CTLA4, FANK1, HAVCR1, CD25, GITR, LAG3, CD127 analytes, a TH1 cell comprising two or more of CD4, CD3D, S100A4, IL7R, IFNG analytes, a TH2 cell comprising two or more of CD4, IL7R, ICOS, CTLA4, TNFRSF4, TNFRS18 analytes, a TH17 cell comprising two or more of CD4, CD3D, IL17A, GZMA, S100A4 analytes, a cytotoxic T cell comprising two or more of CD8, CD3D, S00A4, IFNG, GZMB, GZMA, IL2RB analytes, a plasma B cell comprising two or more JCHAIN, IGLC1, IGHA1, IGHG1, IGKD analytes, a monocyte comprising CD14, CD16 analytes, a natural kill cell comprising NKG7 and NCAM1 analytes, non-plasma B cell CD79A, CD79B analytes, tumor infiltration B cell comprising two or more of MZB1, IGLL5, IGHA1, IGHG1, JCHAIN, IGKC, IGHA2, IGLC2, IGLV3-1, IGLV2-14 analytes, an IgB cell comprising two or more of IGHV3-74, SOCS3, JCHAIN, SPARC analytes, an activated B cell comprising two or more of CD79B, HMGB2, HMGB1, HMGN1, RGS13 analytes, a B cell comprising two or more of MEF2B, RGS13, MS4A1, CD79A, CD79B analytes, or any combinations thereof.

In some aspects, methods herein can be used to determine the presence or absence of prostate cancer in a subject, comprising: a. determining the expression level of two or more captured analytes or complements thereof from a biological sample from the subject, wherein the captured analytes or complements thereof were hybridized to capture probes on a spatial gene expression array, wherein the spatial gene expression array comprises a plurality of capture probes, a capture probe of the plurality comprising a spatial barcode and a capture domain, wherein the two or more captured analytes or complements thereof are from one of groups (i), (ii), (iii), or (iv) or a combination thereof: (i) S100B, TGM4, SMOC1, LTF, ANPEP, SELE, PSCA, MT1G, ACTG2, WFDC2, SOCS3, PCP4, SYNM, GADD45B, TNC, GAS1, CNN1, ZNF185, DES, HSPB6, MSMB, MYLK, IGHG1, FLNC, ADAMTS4, MYL9, ADAM33, KRT5, PGM5, IGHV3-35, NEFH, JPH2, KRT15, TRIM29, SRD5A2, HSPB8, C11orf96, SPEG, ALDH2, FLNA, OLFM4, IGKV1D-12, TAGLN, TPM2, CRYAB, SMTN, KCNMB1, PMP22, ACTA2, and SYNPO2, or a fragment thereof; (ii) ADGRF1, OR51C1P, KCNC2, PLA2G7, SPON2, CAMK2N1, KCNN2, OR51E2, AGTR1, GRIN3A, KHDRBS3, KCNH8, ATP8A2, TMEM26, LUZP2, HPN, PLA2G2A, COL2A1, UNC80, DNASE2B, HOXC6, SPOCK1, AKR7A3, LRRN1, GOLM1, GJB1, FGFRL1, GHR, ACSM3, MCCC2, TRGV9, ZNF385B, CYP39A1, SEC11C, H2AFJ, MYO6, GPR160, BMPR1B, MBOAT2, GALNT3, CPNE4, ACER3, PLEKHH1, C9orf152, and PAQR6, or a fragment thereof; (iii) OR51E1, FGFR3, SPON2, KCNG3, OR51C1P, ARG2, DEGS1, OR51E2, TRGJP2, CPNE4, GOLM1, RDH11, ACSM1, PLEKHH1, SLC30A4, TRGC1, GDF11, KHDRBS3, SPOCK1, GDF15, FGFRL1, TRGV9, LRRN1, PLA2G7, TMSB15A, KCNN2, MARC1, PTPRT, C5orf30, COLEC12, HPN, MYO6, ADGRF1, UBE2E3, HGD, NSD2, KIF21A, HIST1H2AC, LETM1, COL9A2, BEND4, GJB1, ARFGEF3, PRAC1, F3, and SAMD5, or a fragment thereof; (iv) IGHA1, JCHAIN, CD79A, IGHJ6, IGKC, SAA2, CCL19, IGLC1, SAA1, IGHG2, POU2AF1, DERL3, S100A9, IGHG1, LTB, C3, MZB1, IGKV4-1, RARRES1, LTF, IGHG3, IGHJ2, IGLV3-1, LYZ, LSP1, OLFM4, FCGBP, CFB, PDZK1IP1, KRT7, CORO1A, IL7R, PRDM1, CYBA, IGHM, LCN2, SERPINF1, HCLS1, CD53, PTPRC, PTGDS, NNMT, IGFBP4, WFDC2, CP, ITGB2, C1R, CLU, KRT15, and FHL2, or a fragment thereof; and b. identifying the presence or absence of prostate cancer in the subject based on the determined expression level of the two or more analytes or complements thereof.

In some aspects, the methods herein provide for the monitoring of a treatment regimen in a subject with prostate cancer, comprising contacting a biological sample with a spatial gene expression array and determining the expression level of two or more analytes of one or more of the groups (i) through (iv) in the biological sample from the subject, and correlating the determined expression level of the one or more analytes with the stage of prostate cancer in the subject, and proceeding or having proceeded with one or more of (i) administering a prostate cancer treatment to the subject based on the determined expression level of the two or more analytes, (ii) adjusting the dosage of a current prostate cancer treatment based on the determined expression level of the two or more analytes, or (iii) adjusting a prostate cancer treatment based on the determined expression level of the one or more analytes, re-assaying a subject at a later time point following one of the treatment regimens from (i), (ii) or (iii) for changes in gene expression levels, and proceeding with one or more of administering an additional treatment, adjusting the original treatment, or ceasing the treatment regimen, based on the re-assayed expression levels of the two or more analytes correlated with the stage of prostate cancer. In some aspects, luminal cell biomarkers as previously described can be used for monitoring and adjusting, if necessary, a treatment regimen for prostate cancer. In some aspects, basal cell markers such as TP63, KRT5, KRT14, or combinations thereof, can be used for monitoring a treatment regimen. In some aspects, immune cell analytes as previously described can be used for monitoring a treatment regimen. In some aspects, monitoring a treatment regimen in a subject can include determining the expression level of two or more of luminal cell, basal cell, and or immune cell analytes, or any combinations thereof, as previously described.

In some aspects, the methods described herein can be used to determine the presence or absence of invasive prostate cancer in a subj ect comprising contacting or having contacted the biological sample from a subject with a spatial gene expression array, determining or having determined the expression level and/or spatial location of two or more luminal cell analytes as previously described, determining or having determined the expression level and/or spatial location of two or more basal cell analytes as previously described, and correlating the determined expression levels for luminal and basal cell markers with the presence or absence of invasive prostate cancer in a subject.

In some aspects, the methods described herein can be used to determine the presence or absence of invasive prostate cancer in a subject comprising: a. determining the gene expression levels of two or more captured analytes or complements thereof associated with a luminal cell in a biological sample from the subject, wherein the two or more captured analytes or complements thereof associated with a luminal cell were hybridized to capture probes on a spatial gene expression array, wherein the spatial gene expression array comprises a plurality of capture probes, a capture probe of the plurality comprising a spatial barcode and a capture domain, wherein the two or more captured analytes associated with a luminal cell comprise CD24 or a fragment thereof, KRT8 or a fragment thereof, and KRT18 or a fragment thereof, or combinations thereof; b. determining the gene expression levels of two or more captured analytes or complements thereof associated with a basal cell in one or more locations in the biological sample, wherein the two or more captured analytes or complements thereof associated with a basal cell were hybridized to capture probes on the spatial gene expression array, wherein the two or more captured analytes associated with a basal cell comprise TP63, KRT5, KRT14, or combinations thereof; and c. identifying the presence or absence of invasive prostate cancer in the subject based on the determined gene expression levels of the two or more captured analytes or complements thereof associated with a luminal cell and the two or more captured analytes or complements thereof associated with a basal cell.

In some aspects, any of the methods described herein can further comprise assaying serially obtained biological samples from the subject at a plurality of time points and determining the expression level or the two or more analytes in the serially obtained biological samples.

In some aspects, the prostate cancer that is correlated with the expression level of two or more analytes from a sample from a subject is an adenocarcinoma, acinar cell carcinoma, a ductal adenocarcinoma, a transitional cell carcinoma, a squamous cell carcinoma or a small cell prostate carcinoma.

In some aspects, a biological sample can be further stained prior to determining the sequence of an analyte, for example by a histological or immunofluorescence staining method for identifying or having identified one or more cancerous regions in the biological sample prior to any administering and/or adjusting of any dosage of a prostate cancer treatment or medicament for a patient, using any of the methods described herein. For example, H&E histological stains, immunofluorescence stains, and the like are useful it identifying tissue structures and are used by pathologists for annotating biopsies for example. Further, acquiring stained images of prostate samples are used to correlate the locations of the analytes in a sample after the sequences of the analytes and associated spatial barcodes are determined, as described herein. Combinations of tissue staining can also be performed, for example a sample could be stained with H&E in combination with immunofluorescence stains. Additional optical labels which may be of use in sample imaging methods includes chemiluminescent, colorimetric labels, radioactivity, or combinations thereof.

In some aspects, determining the expression level of one or more analytes and/or the spatial location of the one or more analytes, comprises contacting a biological sample such as a tissue section with the spatial gene expression array, wherein the spatial array comprises capture probes that include a spatial barcode and a capture domain, applying RNA templated ligation (RTL) probes to the sample, allowing the RTL probes to hybridize to the analyte, ligating the RTL probes to generate a ligation product that serves as a proxy for the analyte, releasing the ligated ligation product from the analyte for example by enzymatic digestion (e.g. RNase H digestion), permeabilizing the tissue section thereby allowing for the capture of the ligation product by the capture domain, extending the capture domain using the ligation product as a template, amplifying the extended capture probe to produce a plurality of amplified nucleic acids that include a spatial barcode and a sequence present in the analyte or a complement thereof, determining the sequence of the spatial barcode or a complement thereof and the sequence of the analyte or a complement thereof, and using that information to determine the presence and abundance of the analyte and correlate the analyte to a location in the tissue section based on the location and sequence of the spatial barcode.

In some aspects, the analyte measured in the methods described herein is a nucleic acid, such as a mRNA and the expression level and/or location of the mRNA in the tissue is determined. In some aspects, the analyte measured in the methods described herein is a protein and the expression level and/or location of the protein is determined.

In some aspects, determining the expression level of any analyte, or copy, or proxy thereof comprises sequencing, such as sequence by synthesis, sequence by ligation, sequence by hybridization, nanopore sequencing as examples.

In some aspects, the biological samples used in methods described herein include tissue samples for example from a biopsy. The tissue sample can be further sectioned, for example via microtome, into tissue sections that are contacted with a spatial array as described herein. The samples can be fresh, frozen or fixed, for example a sample can be formalin fixed and paraffin embedded prior to contacting with a spatial array of the present methods. Further, the tissue samples can be serial tissue sections or tissue sections from serial biopsies, etc. In some aspects, the biological sample is or was disposed on a spatial gene expression array or on a substrate without the spatial gene expression array. In some aspects, the biological sample that is or was disposed on a substrate without the spatial gene expression array was aligned with a spatial gene expression array, such that at least a portion of the biological sample was aligned with at least a portion of the spatial gene expression array. In some aspects, one or more analytes, or proxies thereof, are migrated from a biological sample disposed on a substrate to the spatial gene expression array with which it is aligned, such that the migrated analytes, or proxies thereof, are captured by the capture probes on the gene expression spatial array. Gene expression analysis can be carried out from the gene expression array, and data correlated back to its relative location in the biological sample on the substrate. Methods for such migration, capture and gene expression analysis can be found in, for example, WO 2020/123320, which is incorporated herein by reference in its entirety.

In some aspects, a prostate sample used in methods described herein have a Gleason score of less than 6 which is typically indicative of a non-cancerous tissue, or a Gleason score of 7 or higher which is typically indicative of a prostate tissue which contains a cancer. In some aspects, a stage of prostate cancer is determined based on the correlation of analyte expression levels and/or their location in a tissue sample as described herein.

In some aspects, methods to determine the gene expression and/or location of an analyte, two or more analytes, in a sample from a subject can be useful in determining the type of treatment to provide a patient. Examples of treatments include drug regimens (e.g., chemotherapy, immunotherapy, bone modifying compounds, surgery, radiation therapy, cryosurgery, high intensity focused ultrasound, hormonal therapy (e.g., luteinizing hormone releasing hormone antagonists, androgen synthesis inhibitors, androgen receptor inhibitors, etc.), targeted therapies (e.g., olaparib or recaparib), or any combination thereof depending on the stage of prostate cancer as correlated with the expression levels of two or more analytes as previously listed. In some aspects, the frequency of treatment regimens and their administration is based on the expression level of the two or more analytes as previously described.

In some aspects of the methods described herein, the prostate sample, cancerous or non-cancerous, is obtained from a human patient, and the expression levels of the two or more analytes as previously described can be used to diagnose, prognose, determine a treatment regimen, and/or monitor a treatment regimen that is personalized to that patient.

In some aspects, the determining the expression levels of any of the two or more analytes from a prostate tissue sample further comprises generating a data set that includes, but is not limited to, analyte expression data, spatial barcode data associated with the analytes, image data from histological or immunofluorescence images, and registration data (e.g., fiducial data) linking the analyte data to the image data, and using the dataset to identify a pre-cancerous region, a cancerous region, the location of different cell types within the sample (e.g., luminal cells, basal cells, immune cells, etc.), or combinations thereof that can be used in the diagnosis, prognosis, determining a treatment regimen, monitoring a treatment regimen or a combination thereof for the patient from which the sample originates.

In some aspects, the present disclosure provides kits for practicing any of the methods described herein, for example a kit can comprise a stain (e.g., histological, immunofluorescent), a spatial gene expression array comprising a capture probe that includes a spatial barcode and a capture domain, a cell permeabilization reagent, RNA templated ligation probes capable of hybridizing to two or more analytes from previously described groups (i), (ii), (iii) (iv), immune cell analytes, luminal cell analytes, and/or basal cell analytes, and instructions for practicing any of the methods described herein. A kit can further include additional reagents that might be needed, for example enzymes, buffers, and the like, for practicing the instructions.

In some aspects, the present disclosure provides a composition that is used for identifying the presence or absence of prostate cancer in a subject, the composition comprising a substrate comprising capture probes that include a spatial barcode and a capture domain, a tissue overlaid on the substrate and two or more templated ligation probe sets that are hybridized to analytes within the tissue sample, wherein the analytes are indicative of the presence or absence of prostate cancer. In some aspects, the two or more probe sets target two or more analytes from previously defined groups (i), (ii), (iii), (iv), immune cells, luminal cells, and/or basal cell analytes. In some aspects, the probe sets target two or more of CD24, KRT8, KRT18, or combinations thereof, two or more of CD3D, CD3E, CD4, CD8A, CD247, CD79A, CD79B, IGHA1, IGHG1, JCHAIN, IGKC, IGLC1, or combinations thereof, two or more of TP63, KRT5, KRT14 or combinations thereof, two or more of CD4, FOXP3, IL17RB, CTLA4, FANK1, HAVCR1, CD24, GITR, LAG3, CD127 or combinations thereof, two or more targets of CD4, CD3D, S100A4, IL7R, IFNG or combinations thereof, two or more targets of CD4, IL7R, ICOS, CTLA4, TNFRSF4, TNFRS18 or combinations thereof, two or more targets of CD4, CD3D, IL17A, GZMA, GZMB, IL2RB or combinations thereof.

In some aspects, this disclosure further provides a system comprising a storage element operable to store a dataset of a plurality of biological samples, wherein the dataset comprises, for each biological sample, 1) analyte data for a plurality of analytes as previously identified captured at a plurality of spatial locations for a reference biological sample and a prostate biological sample, 2) image data for both the reference and a prostate biological sample, 3) registration data for the images, 4) linking data that correlates the image, registration, and analyte data as to the level of gene expression of the analyte and the spatial location in the tissue from which the analyte originated, and 5) a processor operable to process the dataset through a machine learning module to train the machine learning module to determine the presence of absence of prostate cancer in the prostate biological sample, to determine a course of treatment for a subject having prostate cancer, and/or for monitoring the effectiveness of a defined treatment regimen in a subject undergoing treatment for prostate cancer.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 presents a schematic representation of a barcoded capture probe in accordance with the present disclosure.

FIG. 2A-FIG. 2B present images of prostate cancer tissue samples in accordance with Example 1. FIG. 2A presents an image of graph-based spot clustering analysis of gene expression in a tissue section from a non-cancerous prostate tissue sample overlaid with a pathologist's annotation of the same non-cancerous prostate tissue sample in accordance with Example 1. FIG. 2B presents an image of graph-based spot clustering analysis of gene expression in a tissue section from an adenocarcinoma with invasive carcinoma prostate cancer tissue sample overlaid with a pathologist's annotation of the same invasive carcinoma prostate cancer tissue sample in accordance with Example 1.

FIG. 3A-FIG. 3B present images of an adenocarcinoma prostate tissue sample in accordance with Example 1. FIG. 3A presents an image of an adenocarcinoma prostate tissue sample with pathologist annotations in accordance with Example 1. FIG. 3B presents an image of K-means (K=2) clustering analysis of gene expression in the adenocarcinoma prostate tissue section in accordance with Example 1. In FIG. 3B, K=2 clusters.

FIG. 4A-FIG. 4C present images of an acinar carcinoma prostate tissue sample in accordance with Example 1. FIG. 4A presents an image of acinar carcinoma prostate tissue sample with pathologist annotations in accordance with Example 1. FIG. 4B presents an image of K-means (K=9) clustering analysis of gene expression showing nine clusters in accordance with Example 1. FIG. 4C presents an image of K-means (K=2) clustering analysis of gene expression showing two clusters in accordance with Example 1. Cluster 1 represents tumorous regions of the sample and cluster 2 represents a non-tumorous region.

FIG. 5A-FIG. 5E present images and analysis related to an acinar carcinoma prostate tissue sample analyzed in accordance with Example 1. FIG. 5A presents a violin plot of the relative expression level of AMACR in Cluster 1 and Cluster 2 in accordance with Example 1 and FIG. 4C. FIG. 5B presents a violin plot of the relative expression level of ERG in Cluster 1 and Cluster 2 in accordance with Example 1 and FIG. 4C. FIG. 5C presents a violin plot of the relative expression level of NPY in Cluster 1 and Cluster 2 in accordance with Example 1 and FIG. 4C. FIG. 5D presents a violin plot of the relative expression level of CRISP3 in Cluster 1 and Cluster 2 in accordance with Example 1 and FIG. 4C. FIG. 5E presents a violin plot of the relative expression level of TMEFF2 in Cluster 1 and Cluster 2 in accordance with Example 1 and FIG. 4C.

FIG. 6A-FIG. 6C present graphical representations of gene expression of AMACR (UMI counts shown) in various different prostate tissue samples in accordance with Example 1. FIG. 6A presents an image of graph-based spot clustering analysis of gene expression of AMACR in a normal, non-cancerous prostate tissue sample in accordance with Example 1. FIG. 6B presents an image of graph-based spot clustering analysis of gene expression of AMACR in an adenocarcinoma with invasive carcinoma prostate tissue sample in accordance with Example 1. FIG. 6C presents an image of graph-based spot clustering analysis of gene expression of AMACR in an acinar cell carcinoma prostate tissue sample in accordance with Example 1.

FIG. 7A-FIG. 7C present images of gene expression of ERG in various different prostate tissue samples in accordance with Example 1. FIG. 7A presents an image of gene expression of ERG in a normal, non-cancerous prostate tissue sample in accordance with Example 1. FIG. 7B presents an image of gene expression of ERG in an adenocarcinoma with invasive carcinoma prostate tissue sample in accordance with Example 1. FIG. 7C presents an image of gene expression of ERG in an acinar cell carcinoma prostate tissue sample in accordance with Example 1.

FIG. 8A-FIG. 8D present images of gene expression showing gene expression and colocalization of basal cells (FIG. 8A and FIG. 8C) and luminal cells (FIG. 8B and FIG. 8D) in normal, non-cancerous prostate tissue sample (FIG. 8A and FIG. 8B) or an adenocarcinoma with invasive carcinoma prostate tissue sample (FIG. 8C and FIG. 8D) in accordance with Example 1. FIG. 8A presents an image of gene expression of basal cells in a normal, non-cancerous prostate tissue sample in accordance with Example 1. FIG. 8B presents an image of gene expression of luminal cells in a normal, non-cancerous prostate tissue sample in accordance with Example 1. FIG. 8C presents an image of gene expression of basal cells in an adenocarcinoma with invasive carcinoma prostate tissue sample in accordance with Example 1. FIG. 8D presents an image of gene expression of luminal cells in an adenocarcinoma with invasive carcinoma prostate tissue sample in accordance with Example 1.

FIG. 9A-FIG. 9F present images of T cell gene expression and spatial distribution of the T cells in accordance with Example 1. FIG. 9A presents an image of T cell gene expression in a normal, non-cancerous prostate tissue sample in accordance with Example 1. FIG. 9B presents a violin plot of T cell gene expression in a normal, non-cancerous prostate tissue sample accordance with Example 1. FIG. 9C presents an image of gene expression in an adenocarcinoma with invasive carcinoma prostate tissue sample in accordance with Example 1. FIG. 9D presents a violin plot of T cell gene expression in an adenocarcinoma with invasive carcinoma prostate tissue sample accordance with Example 1. FIG. 9E presents an image of gene expression in an acinar cell carcinoma prostate tissue sample in accordance with Example 1. FIG. 9F presents a violin plot of T cell gene expression in an acinar cell carcinoma prostate tissue sample accordance with Example 1.

FIG. 10A-FIG. 10C present images of gene expression and spatial distribution of plasma B cells in accordance with Example 1. FIG. 10A presents an image of gene expression and spatial distribution of plasma B cells in a normal, non-cancerous prostate tissue sample in accordance with Example 1. FIG. 10B presents an image of gene expression and spatial distribution of plasma B cells in an adenocarcinoma with invasive carcinoma prostate tissue sample in accordance with Example 1. FIG. 10C presents an image of gene expression and spatial distribution of plasma B cells in an acinar cell carcinoma prostate tissue sample in accordance with Example 1.

FIG. 11A-FIG. 11B present images of B cell gene expression in tissue samples in accordance with Example 1. FIG. 11A presents an image of B cell gene expression in a normal, non-cancerous prostate tissue sample in accordance with Example 1. FIG. 11B presents an image of B cell gene expression in an adenocarcinoma with invasive carcinoma prostate tissue sample in accordance with Example 1.

FIG. 12A-FIG. 12F present images of comparisons of immune cell gene expression in normal, non-cancerous prostate tissue samples or in an adenocarcinoma prostate tissue sample in accordance with Example 1. FIG. 12A presents an image of gene expression of TAM immune cells in a normal, non-cancerous prostate tissue sample in accordance with Example 1. FIG. 12B presents an image of gene expression of TAM immune cells in an adenocarcinoma prostate tissue sample in accordance with Example 1. FIG. 12C presents an image of gene expression of CD14, preferentially expressed on monocytes and macrophage immune cells, in a normal, non-cancerous prostate tissue sample in accordance with Example 1. FIG. 12D presents an image of gene expression of CD14 in an adenocarcinoma prostate tissue sample in accordance with Example 1. FIG. 12E presents an image of gene expression of NK immune cells in a normal, non-cancerous prostate tissue sample in accordance with Example 1. FIG. 12F presents an image of gene expression of NK immune cells in an adenocarcinoma prostate tissue sample in accordance with Example 1.

FIG. 13A-FIG. 13F present images of IHC staining for AMACR protein expression in various prostate tissue samples in accordance with Example 2. FIG. 13A presents an image of IHC staining for AMACR protein expression in a normal, non-cancerous prostate tissue sample in accordance with Example 2. FIG. 13B presents the inset of the image of FIG. 13A, which presents IHC staining for AMACR protein expression in a normal, non-cancerous prostate tissue sample in accordance with Example 2. FIG. 13C presents an image of IHC staining for AMACR protein expression in an adenocarcinoma with invasive carcinoma prostate tissue sample in accordance with Example 2. FIG. 13D presents the inset of the image of FIG. 13C, which presents an image of IHC staining for AMACR protein expression in an adenocarcinoma with invasive carcinoma prostate tissue sample in accordance with Example 2. FIG. 13E presents an image of IHC staining for AMACR protein expression in an acinar cell carcinoma prostate tissue sample in accordance with Example 2. FIG. 13F presents the inset of the image of FIG. 13E, which presents an image of IHC staining for AMACR protein expression in an acinar cell carcinoma prostate tissue sample in accordance with Example 2.

FIG. 14A-FIG. 14F present images of IHC staining for ERG protein expression in various different prostate tissue samples in accordance with Example 2. FIG. 14A presents an image of IHC staining for ERG protein expression in a normal, non-cancerous prostate tissue sample in accordance with Example 2. FIG. 14B presents the inset of the image of FIG. 14A, which presents IHC staining for ERG protein expression in a normal, non-cancerous prostate tissue sample in accordance with Example 2. FIG. 14C presents an image of IHC staining for ERG protein expression in an adenocarcinoma with invasive carcinoma prostate tissue sample in accordance with Example 2. FIG. 14D presents the inset of the image of FIG. 14C, which presents an image of IHC staining for ERG protein expression in an adenocarcinoma with invasive carcinoma prostate tissue sample in accordance with Example 2. FIG. 14E presents an image of IHC staining for ERG protein expression in an acinar cell carcinoma prostate tissue sample in accordance with Example 2. FIG. 14F presents the inset of the image of FIG. 14E, which presents an image of IHC staining for ERG protein expression in an acinar cell carcinoma prostate tissue sample in accordance with Example 2.

### DETAILED DESCRIPTION

### 1. Terminology

As used in the present disclosure and claims, the singular forms “a,” “an,” and “the” include plural forms unless the context clearly dictates otherwise.

It is understood that wherever aspects are described herein with the language “comprising,” otherwise analogous aspects described in terms of “consisting of” and/or “consisting essentially of” are also provided. In this disclosure, “comprises,” “comprising,” “containing” and “having” and the like can mean “includes,” “including,” and the like; “consisting essentially of” or “consists essentially of” are open-ended, allowing for the presence of more than that which is recited so long as basic or novel characteristics of that which is recited is not changed by the presence of more than that which is recited, but excludes prior art aspects.

Unless specifically stated or obvious from context, as used herein, the term “or” is understood to be inclusive. The term “and/or” as used in a phrase such as “A and/or B” herein is intended to include both “A and B,” “A or B,” “A,” and “B.” Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

As used herein, the terms “about” and “approximately,” when used to modify a numeric value or numeric range, indicate that deviations of up to 10% above and down to 10% below the value or range remain within the intended meaning of the recited value or range. It is understood that wherever aspects are described herein with the language “about” or “approximately” a numeric value or range, otherwise analogous aspects referring to the specific numeric value or range are also provided.

A “splint oligonucleotide” is an oligonucleotide that, when hybridized to other polynucleotides, acts as a “splint” to position the polynucleotides next to one another so that they can be ligated together. In some embodiments, the splint oligonucleotide is DNA or RNA, or a combination thereof. The splint oligonucleotide can include a nucleotide sequence that is partially complimentary to nucleotide sequences from two or more different polynucleotides. In some embodiments, the splint oligonucleotide assists in ligating a “donor” oligonucleotide and an “acceptor” oligonucleotide. In general, an RNA ligase, a DNA ligase, or another other variety of ligase is used to ligate two splinted nucleotide sequences together.

The terms “nucleic acid” and “nucleotide” are intended to be consistent with their use in the art and to include naturally-occurring species or functional analogs thereof. Particularly useful functional analogs of nucleic acids are capable of hybridizing to a nucleic acid in a sequence-specific fashion (e.g., capable of hybridizing to two nucleic acids such that ligation can occur between the two hybridized nucleic acids) or are capable of being used as a template for replication of a particular nucleotide sequence. Naturally-occurring nucleic acids generally have a backbone containing phosphodiester bonds. An analog structure can have an alternate backbone linkage including any of a variety of those known in the art. Naturally-occurring nucleic acids generally have a deoxyribose sugar (e.g., found in deoxyribonucleic acid (DNA)) or a ribose sugar (e.g., found in ribonucleic acid (RNA)). A nucleic acid can contain nucleotides having any of a variety of analogs of these sugar moieties that are known in the art. A nucleic acid can include native or non-native nucleotides. In this regard, a native deoxyribonucleic acid can have one or more bases selected from the group consisting of adenine (A), thymine (T), cytosine (C), or guanine (G), and a ribonucleic acid can have one or more bases selected from the group consisting of uracil (U), adenine (A), cytosine (C), or guanine (G). Useful non-native bases that can be included in a nucleic acid or nucleotide are known in the art.

A “probe” or a “target,” when used in reference to a nucleic acid or sequence of a nucleic acids, is intended as a semantic identifier for the nucleic acid or sequence in the context of a method or composition, and does not limit the structure or function of the nucleic acid or sequence beyond what is expressly indicated.

The terms “oligonucleotide” and “polynucleotide” are used interchangeably to refer to a single-stranded multimer of nucleotides, for example from about 2 to about 500 nucleotides in length. Oligonucleotides can be synthetic, made enzymatically (e.g., via polymerization), or using a “split-pool” method. Oligonucleotides can include ribonucleotide monomers (i.e., can be oligoribonucleotides) and/or deoxyribonucleotide monomers (i.e., oligodeoxyribonucleotides). In some examples, oligonucleotides can include a combination of both deoxyribonucleotide monomers and ribonucleotide monomers in the oligonucleotide (e.g., random or ordered combination of deoxyribonucleotide monomers and ribonucleotide monomers). An oligonucleotide can be 4 to 10, 10 to 20, 21 to 30, 31 to 40, 41 to 50, 51 to 60, 61 to 70, 71 to 80, 80 to 100, 100 to 150, 150 to 200, 200 to 250, 250 to 300, 300 to 350, 350 to 400, or 400-500 nucleotides in length, for example. Oligonucleotides can include one or more functional moieties that are attached (e.g., covalently or non-covalently) to the multimer structure. For example, an oligonucleotide can include one or more detectable labels (e.g., a radioisotope or fluorophore).

A “subject” generally refers to an animal, such as a mammal (e.g., human or a non-human simian). In some aspects, a subject can be a mammal. In some aspects, a mammal can be a mouse. In some aspects, a mammal can be a rat. In some aspects, a mammal can be a nonhuman primate, such as a chimpanzee, a gorilla, an orangutan, a rhesus monkey, a cynomolgus monkey, a Taiwanese macaque, a green monkey, a squirrel monkey, tamarin, a marmoset, or a mouse lemur. In some aspects, a mammal can be a human. In some aspects, a subject can be an animal model of prostate cancer. In some aspects, a mammal can be a mammalian model of prostate cancer. In some aspects, an animal model of prostate cancer can express one or more human genes.

An “adaptor,” an “adapter,” and a “tag” are terms that are used interchangeably in this disclosure, and refer to species that can be coupled to a polynucleotide sequence (in a process referred to as “tagging”) using any one of many different techniques including (but not limited to) ligation, hybridization, and tagmentation. Adaptors can also be nucleic acid sequences that add a function, e.g., spacer sequences, primer sequences/sites, barcode sequences, unique molecular identifier sequences.

The terms “hybridizing,” “hybridize,” “annealing,” and “anneal” are used interchangeably in this disclosure, and refer to the pairing of substantially complementary or complementary nucleic acid sequences within two different molecules. Pairing can be achieved by any process in which a nucleic acid sequence joins with a substantially or fully complementary sequence through base pairing to form a hybridization complex. For purposes of hybridization, two nucleic acid sequences are “substantially complementary” if at least 60% (e.g., at least 70%, at least 80%, or at least 90%) of their individual bases are complementary to one another.

The terms “detectable label,” “optical label,” and “label” are used interchangeably herein to refer to a directly or indirectly detectable moiety that is associated with (e.g., conjugated to) a molecule to be detected, e.g., a capture probe or analyte. The detectable label can be directly detectable by itself (e.g., radioisotope labels or fluorescent labels) or, in the case of an enzymatic label, can be indirectly detectable, e.g., by catalyzing chemical alterations of a chemical substrate compound or composition, which chemical substrate compound or composition is directly detectable. Detectable labels can be suitable for small scale detection and/or suitable for high-throughput screening. As such, suitable detectable labels include, but are not limited to, radioisotopes, fluorophores, chemiluminescent compounds, bioluminescent compounds, and dyes.

The term “presence” as used herein refers to the existence and/or level(s) of any object(s) (e.g., an analyte) being measured, quantified, and/or compared in any of the methods described herein.

The terms “administer,” “administering,” “administration,” and the like, as used herein, refer to methods that may be used to deliver a drug to the desired site of biological action. Administration techniques that can be employed with the agents and methods described herein are found in e.g., Goodman and Gilman, The Pharmacological Basis of Therapeutics, current edition, Pergamon; and Remington's, Pharmaceutical Sciences, current edition, Mack Publishing Co., Easton, Pa.

The term “therapeutically effective amount” refers to an amount of a drug effective to treat a disease or condition in a subject. In the case of cancer, the therapeutically effective amount of the drug can reduce the number of cancer cells; reduce the tumor size or burden; inhibit (i.e., slow to some extent and in some aspects, stop) cancer cell infiltration into peripheral organs; inhibit (i.e., slow to some extent and in some aspects, stop) tumor metastasis; inhibit, to some extent, tumor growth; inhibit, to some extent, angiogenesis; relieve to some extent one or more of the symptoms associated with the cancer; and/or result in a favorable response such as increased progression-free survival (PFS), disease-free survival (DFS), or overall survival (OS), complete response (CR), partial response (PR), or, in some cases, stable disease (SD), a decrease in progressive disease (PD), a reduced time to progression (TTP), or any combination thereof. To the extent the drug can prevent growth and/or kill existing cancer cells, it can be cytostatic and/or cytotoxic.

Terms such as “treating” or “treatment” or “to treat” or “alleviating” or “to alleviate” refer to therapeutic measures that cure, slow down, lessen symptoms of, and/or halt progression of a diagnosed pathologic condition or disorder. Thus, those in need of treatment include those already diagnosed with or suspected of having the disorder. In some aspects, a subject is successfully “treated” for cancer according to the methods provided herein if the patient shows one or more of the following: a reduction in the number of or complete absence of cancer cells; a reduction in tumor size; inhibition of or an absence of cancer cell infiltration into peripheral organs including, for example, the spread of cancer into soft tissue and bone; inhibition of or an absence of tumor metastasis; inhibition or an absence of tumor growth; relief of one or more symptoms associated with the specific cancer; reduced morbidity and mortality; improvement in quality of life; reduction in tumorigenicity, tumorigenic frequency, or tumorigenic capacity, of a tumor; reduction in the number or frequency of cancer stem cells in a tumor; differentiation of tumorigenic cells to a non-tumorigenic state; increased progression-free survival (PFS), disease-free survival (DFS), or overall survival (OS), complete response (CR), partial response (PR), stable disease (SD), a decrease in progressive disease (PD), a reduced time to progression (TTP), or any combination thereof.

### 2. Introduction

As discussed supra, methods of accurately detecting and diagnosing prostate cancer are needed in the field. For instance, methods for diagnosing the presence and/or stage of prostate cancer are subjective and often suffer from false positive or false negative results. Such methods include PSA tests, digital rectal exams (DREs), and pathological analysis of a prostate tissue biopsy. Though prostate biopsies are generally used for an actual diagnosis of prostate cancer, the biopsies, too, are a subjective method that can suffer from false negatives or false positives. For instance, the pathologist's annotations depend on the pathologist visually inspecting a stained sample and grading it on a subjective scale to produce a Gleason score, which is an indicator of the severity of the prostate cancer. While more accurate than PSA tests and/or DRE, false positives and false negatives occur using pathological analysis. As discussed supra, early detection of prostate cancer is critical for subjects as early detection can provide patients with multiple treatment options that may not be available for more advanced disease stages. Moreover, accurate staging of prostate cancer is critical for administration of the appropriate treatment, as discussed further infra.

Therefore, provided herein are methods of determining the expression level of one or more analytes in a subject, identifying the expression level of the one or more analytes and correlating the identified expression level to a stage of prostate cancer, or to a likelihood of developing prostate cancer. The present disclosure includes aspects related to administering a prostate cancer treatment to the subject based on the expression level of the one or more analytes; adjusting a dosage of a current prostate cancer treatment for the subject based on the expression level of the one or more analytes; or adjusting a prostate cancer treatment for the subject based on the expression level of the one or more analytes. The present disclosure further includes aspects related to administering a prophylactic prostate cancer treatment to the subject based on the expression level of the one or more analytes; adjusting a prophylactic prostate cancer treatment dosage of a current prostate cancer prophylactic treatment for the subject based on the expression level of the one or more analytes; and adjusting a prophylactic prostate cancer treatment for the subject based on the expression level of the one or more analytes.

Moreover, the present disclosure generally relates to methods of determining the expression level of one or more analytes in a subject, identifying the expression level of the one or more analytes and correlating the identified expression level to a stage of prostate cancer or a likelihood of developing prostate cancer, identifying a cancerous or pre-cancerous region of a sample based on the identified expression level of the one or more analytes, identifying one or more luminal cells or an analyte associated with a luminal cell in one or more locations in the biological sample, and determining the degree of luminal cell infiltration in the cancerous or pre-cancerous region of the biological sample obtained from the subject. The present disclosure includes aspects related to administering a prostate cancer treatment to the subject based on the identified expression level of the one or more analytes and the degree of luminal cell infiltration; adjusting a dosage of a current prostate cancer treatment for the subject based on the identified expression level of the one or more analytes and the degree of luminal cell infiltration; adjusting a prostate cancer treatment for the subject based on the identified expression level of the one or more analytes and the degree of luminal cell infiltration; administering a prophylactic prostate cancer treatment to the subject based on the identified expression level of the one or more analytes and the degree of luminal cell infiltration; adjusting a prophylactic prostate cancer treatment of a current prostate cancer prophylactic treatment for the subject based on the identified expression level of the one or more analytes and the degree of luminal cell infiltration; or adjusting a prophylactic prostate cancer treatment for the subject based on the identified expression level of the one or more analytes and the degree of luminal cell infiltration.

Furthermore, the present disclosure generally relates to methods of determining the expression level of one or more analytes in a subject, identifying the expression level of the one or more analytes and correlating the identified expression level to a stage of prostate cancer or a likelihood of developing prostate cancer, identifying a cancerous or pre-cancerous region of a sample based on the identified expression level of the one or more analytes, identifying one or more immune cells or an analyte associated with an immune cell in one or more locations in the biological sample, and determining the degree of immune cell infiltration in the cancerous or pre-cancerous region of the biological sample obtained from the subject. The present disclosure includes aspects related to administering a prostate cancer treatment to the subject based on the identified expression level of the one or more analytes and the degree of immune cell infiltration; adjusting a dosage of a current prostate cancer treatment for the subject based on the identified expression level of the one or more analytes and the degree of immune cell infiltration; adjusting a prostate cancer treatment for the subject based on the identified expression level of the one or more analytes and the degree of immune cell infiltration; administering a prophylactic prostate cancer treatment to the subject based on the identified expression level of the one or more analytes and the degree of immune cell infiltration; adjusting a prophylactic prostate cancer treatment of a current prostate cancer prophylactic treatment for the subject based on the identified expression level of the one or more analytes and the degree of immune cell infiltration; or adjusting a prophylactic prostate cancer treatment for the subject based on the identified expression level of the one or more analytes and the degree of immune cell infiltration.

Moreover, the present disclosure generally relates to methods of preparing a tissue sample, which method comprises obtaining a tissue sample from a subject, staining the sample, adding probes capable of hybridizing to one or more analytes, permeabilizing the cells, and determining the expression level of one or more analytes in a stained tissue sample from a subject. The identified expression level of the one or more analytes can then be correlated with the stage of prostate cancer or likelihood of developing prostate cancer in the sample from the subject.

Furthermore, the present disclosure generally relates to a method for determining the presence or absence of prostate cancer in a subject, comprising: a. contacting a biological sample from a subject with a spatial gene expression array, wherein the spatial gene expression array comprises a plurality of capture probes, a capture probe of the plurality comprising a spatial barcode and a capture domain; b. hybridizing analytes from the biological sample or complements thereof to the capture probes on the spatial gene expression array; c. determining the expression level of two or more captured analytes from the biological sample from one of groups (i), (ii), (iii), or (iv) or a combination thereof: (i) S100B, TGM4, SMOC1, LTF, ANPEP, SELE, PSCA, MT1G, ACTG2, WFDC2, SOCS3, PCP4, SYNM, GADD45B, TNC, GAS1, CNN1, ZNF185, DES, HSPB6, MSMB, MYLK, IGHG1, FLNC, ADAMTS4, MYL9, ADAM33, KRT5, PGM5, IGHV3-35, NEFH, JPH2, KRT15, TRIM29, SRD5A2, HSPB8, C11orf96, SPEG, ALDH2, FLNA, OLFM4, IGKV1D-12, TAGLN, TPM2, CRYAB, SMTN, KCNMB1, PMP22, ACTA2, and SYNPO2, or a fragment thereof; (ii) ADGRF1, OR51C1P, KCNC2, PLA2G7, SPON2, CAMK2N1, KCNN2, OR51E2, AGTR1, GRIN3A, KHDRBS3, KCNH8, ATP8A2, TMEM26, LUZP2, HPN, PLA2G2A, COL2A1, UNC80, DNASE2B, HOXC6, SPOCK1, AKR7A3, LRRN1, GOLM1, GJB1, FGFRL1, GHR, ACSM3, MCCC2, TRGV9, ZNF385B, CYP39A1, SEC11C, H2AFJ, MYO6, GPR160, BMPR1B, MBOAT2, GALNT3, CPNE4, ACER3, PLEKHH1, C9orf152, and PAQR6, or a fragment thereof; (iii) OR51E1, FGFR3, SPON2, KCNG3, OR51C1P, ARG2, DEGS1, OR51E2, TRGJP2, CPNE4, GOLM1, RDH11, ACSM1, PLEKHH1, SLC30A4, TRGC1, GDF11, KHDRBS3, SPOCK1, GDF15, FGFRL1, TRGV9, LRRN1, PLA2G7, TMSB15A, KCNN2, MARC1, PTPRT, C5orf30, COLEC12, HPN, MYO6, ADGRF1, UBE2E3, HGD, NSD2, KIF21A, HIST1H2AC, LETM1, COL9A2, BEND4, GJB1, ARFGEF3, PRAC1, F3, and SAMD5, or a fragment thereof; (iv) IGHA1, JCHAIN, CD79A, IGHJ6, IGKC, SAA2, CCL19, IGLC1, SAA1, IGHG2, POU2AF1, DERL3, S100A9, IGHG1, LTB, C3, MZB1, IGKV4-1, RARRES1, LTF, IGHG3, IGHJ2, IGLV3-1, LYZ, LSP1, OLFM4, FCGBP, CFB, PDZK1IP1, KRT7, CORO1A, IL7R, PRDM1, CYBA, IGHM, LCN2, SERPINF1, HCLS1, CD53, PTPRC, PTGDS, NNMT, IGFBP4, WFDC2, CP, ITGB2, C1R, CLU, KRT15, and FHL2, or a fragment thereof; and (d) determining the presence or absence of prostate cancer in the subject based on the determined expression level of the two or more analytes. In some aspects, the hybridizing comprises: a. providing two or more sets of target analyte probes to the biological sample, wherein a first probe of the set comprises a capture probe binding domain and a second probe of the set comprises a functional sequence, and wherein the first and second probes hybridize adjacent to each other on the target analyte; b. ligating the first and second probe, thereby generated a ligation product; c. releasing the ligation product from the target analyte; d. permeabilizing the biological sample; thereby allowing the ligation product to hybridize to the capture domain of the capture probe on the spatial gene expression array. In some aspects, the determining the expression level of two or more analytes comprises: a. extending the capture probe using the ligation product as a template, thereby generating an extension product that includes the spatial barcode and target analyte sequence, or complements thereof; b. releasing the extended capture probe; c. amplifying the released extended capture probe to generate a plurality of nucleic acids comprising the spatial barcode and target analyte sequences, or complements thereof, and d. sequencing the amplified nucleic acids, thereby determining the expression level of the two or more analytes in the biological sample. In some aspects, sequencing comprises sequence by synthesis, sequence by hybridization, sequence by ligation, or nanopore sequencing. In some aspects, the determining the expression level of two or more analytes comprises: a. determining the expression level of two or more analytes associated with a luminal cell in one or more locations in the biological sample, wherein the one or more analytes is CD24 or a fragment thereof, KRT8 or a fragment thereof, and KRT18 or a fragment thereof, or combinations thereof; and b. correlating the presence or absence of prostate cancer in the subject based on the determined expression level of the analytes associated with the luminal cells.

In some aspects, the determining the expression level of two or more analytes comprises: determining the expression level of two or more analytes associated with an immune cell in one or more locations in the biological sample, wherein the two or more analytes is CD3D or a fragment thereof, CD3E or a fragment thereof, CD4 or a fragment thereof, CD8A or a fragment thereof, CD247 or a fragment thereof; CD79A or a fragment thereof, CD79B or a fragment thereof, IGHAl or a fragment thereof, IGHG1 or a fragment thereof, JCHAIN or a fragment thereof, IGKC or a fragment thereof, IGLC1 or a fragment thereof, or combinations thereof, in the biological sample from the subject; and b. correlating the presence or absence of prostate cancer in the subject based on the determined expression level of the analytes associated with the immune cells.

Moreover, the present disclosure generally relates to a method of monitoring a treatment regimen for prostate cancer in a subject, comprising: a. determining the gene expression levels of two or more captured analytes from the biological sample from one of groups (i), (ii), (iii), or (iv) or a combination thereof, wherein the expression levels of the two or more analytes had been previously determined for the subject prior to being treated for prostate cancer: (i) S100B, TGM4, SMOC1, LTF, ANPEP, SELE, PSCA, MT1G, ACTG2, WFDC2, SOCS3, PCP4, SYNM, GADD45B, TNC, GAS1, CNN1, ZNF185, DES, HSPB6, MSMB, MYLK, IGHG1, FLNC, ADAMTS4, MYL9, ADAM33, KRT5, PGM5, IGHV3-35, NEFH, JPH2, KRT15, TRIM29, SRD5A2, HSPB8, C11orf96, SPEG, ALDH2, FLNA, OLFM4, IGKV1D-12, TAGLN, TPM2, CRYAB, SMTN, KCNMB1, PMP22, ACTA2, and SYNPO2, or a fragment thereof; (ii) ADGRF1, OR51C1P, KCNC2, PLA2G7, SPON2, CAMK2N1, NPY, KCNN2, OR51E2, AGTR1, GRIN3A, KHDRBS3, KCNH8, ATP8A2, TMEM26, LUZP2, HPN, PLA2G2A, COL2A1, UNC80, DNASE2B, HOXC6, SPOCK1, AKR7A3, LRRN1, GOLM1, GJB1, FGFRL1, GHR, ACSM3, MCCC2, TRGV9, ZNF385B, CYP39A1, SEC11C, H2AFJ, MYO6, GPR160, BMPR1B, MBOAT2, GALNT3, CPNE4, ACER3, PLEKHH1, C9orf152, and PAQR6, or a fragment thereof; (iii) OR51E1, FGFR3, SPON2, KCNG3, OR51C1P, ARG2, DEGS1, OR51E2, TRGJP2, CPNE4, GOLM1, RDH11, ACSM1, PLEKHH1, SLC30A4, TRGC1, GDF11, KHDRBS3, SPOCK1, GDF15, FGFRL1, TRGV9, LRRN1, PLA2G7, TMSB15A, KCNN2, MARC1, PTPRT, C5orf30, COLEC12, HPN, MYO6, ADGRF1, UBE2E3, HGD, NSD2, KIF21A, HIST1H2AC, LETM1, COL9A2, BEND4, GJB1, ARFGEF3, PRAC1, F3, and SAMD5, or a fragment thereof; (iv) IGHA1, JCHAIN, CD79A, IGHJ6, IGKC, SAA2, CCL19, IGLC1, SAA1, IGHG2, POU2AF1, DERL3, S100A9, IGHG1, LTB, C3, MZB1, IGKV4-1, RARRES1, LTF, IGHG3, IGHJ2, IGLV3-1, LYZ, LSP1, OLFM4, FCGBP, CFB, PDZK1IP1, KRT7, CORO1A, IL7R, PRDM1, CYBA, IGHM, LCN2, SERPINF1, HCLS1, CD53, PTPRC, PTGDS, NNMT, IGFBP4, WFDC2, CP, ITGB2, C1R, CLU, KRT15, and FHL2, or a fragment thereof; (b) Comparing the determined gene expression levels of the two or more analytes before treatment of the subject with the determined gene expression levels of the two or more analytes during treatment of the subject for prostate cancer and based on that comparison, perform one or more of: (i) administering an additional treatment to the subject based on the comparative difference in the expression levels of the two or more analytes; (ii) adjusting the treatment based on the comparative difference in the expression levels of the two or more analytes; or (iii) ceasing the treatment based on the comparative difference in the expression levels of the two or more analytes. In some aspects, the determining gene expression levels for comparison comprises: a. determining the gene expression levels of two or more analytes associated with a luminal cell in one or more locations in the biological sample, wherein the two or more analytes is CD24 or a fragment thereof, KRT8 or a fragment thereof, and KRT18 or a fragment thereof, or combinations thereof, in the biological sample from the subject; and determining the gene expression levels of two or more analytes associated with a basal cell in one or more locations in the biological sample, wherein the two or more analytes is TP63, KRT5, KRT14, or combinations thereof, in the biological sample from the subject. In some aspects, the determining gene expression levels for comparison comprises determining the gene expression levels of two or more analytes associated with an immune cell in two or more locations in the biological sample, wherein the two or more analytes is CD3D or a fragment thereof, CD3E or a fragment thereof, CD4 or a fragment thereof, CD8A or a fragment thereof, CD247 or a fragment thereof; CD79A or a fragment thereof, CD79B or a fragment thereof, IGHAl or a fragment thereof, IGHG1 or a fragment thereof, JCHAIN or a fragment thereof, IGKC or a fragment thereof, IGLC1 or a fragment thereof. In some aspects, the determining comprises: a. providing two or more sets of target analyte probes to the biological sample, wherein a first probe of the set comprises a capture probe binding domain and a second probe of the set comprises a functional sequence, and wherein the first and second probes hybridize adjacent to each other on the target analyte; b. ligating the first and second probe, thereby generated a ligation product; c. releasing the ligation product from the target analyte; d. permeabilizing the biological sample; e. hybridizing the ligation product to the capture probe on the spatial gene expression array; f. extending the capture probe using the ligation product as a template, thereby generating an extension product that includes the spatial barcode and target analyte sequence, or complements thereof; g. releasing the extended capture probe; h. amplifying the released extended capture probe to generate a plurality of nucleic acids comprising the spatial barcode and target analyte sequences, or complements thereof, and i. sequencing the amplified nucleic acids, thereby determining the expression levels of the two or more analytes in the biological sample prior to and after the subject is treated for prostate cancer. In some aspects, sequencing comprises sequence by synthesis, sequence by hybridization, sequence by ligation, or nanopore sequencing.

Furthermore, the present disclosure generally relates to a method of identifying the presence or absence of invasive prostate cancer in a subject, comprising: a. contacting a biological sample from a subject with a spatial gene expression array, wherein the spatial gene expression array comprises a plurality of capture probes, a capture probe of the plurality comprising a spatial barcode and a capture domain; b. hybridizing analytes from the biological sample or complements thereof to the capture probes on the spatial gene expression array; c. determining the gene expression levels of two or more analytes associated with a luminal cell in the biological sample, wherein the two or more analytes is CD24 or a fragment thereof, KRT8 or a fragment thereof, and KRT18 or a fragment thereof, or combinations thereof; d. determining the gene expression levels of two or more analytes associated with a basal cell in one or more locations in the biological sample, wherein the two or more analytes is TP63, KRT5, KRT14, or combinations thereof; and e. identifying the presence or absence of invasive prostate cancer in the subject based on the determined gene expression levels of the two or more analytes. In some aspects, the hybridizing comprises: a. providing two or more sets of target analyte probes to the biological sample, wherein a first probe of the set comprises a capture probe binding domain and a second probe of the set comprises a functional sequence, and wherein the first and second probes hybridize adjacent to each other on the target analyte; b. ligating the first and second probe, thereby generated a ligation product; c. releasing the ligation product from the target analyte; d. permeabilizing the biological sample; thereby allowing the ligation product to hybridize to the capture domain of the capture probe on the spatial gene expression array. In some aspects, the determining the expression level of two or more analytes comprises: a. extending the capture probe using the ligation product as a template, thereby generating an extension product that includes the spatial barcode and target analyte sequence, or complements thereof; b. releasing the extended capture probe; c. amplifying the released extended capture probe to generate a plurality of nucleic acids comprising the spatial barcode and target analyte sequences, or complements thereof, and d. sequencing the amplified nucleic acids, thereby determining the expression level of the two or more analytes in the biological sample. In some aspects, sequencing comprises sequence by synthesis, sequence by hybridization, sequence by ligation, or nanopore sequencing. In some aspects, the determining the gene expression levels of two or more analytes comprises assaying serially obtained biological samples from the subject at a plurality of time points and determining the expression levels of the two or more analytes in the serially obtained biological samples from the subject. In some aspects, the determining the gene expression levels of the two or more analytes further comprises comparing the gene expression levels of the two or more analytes with the gene expression levels of the two or more analytes from a reference tissue sample. In some aspects, the prostate cancer is adenocarcinoma, acinar cell carcinoma, ductal adenocarcinoma, transitional cell (or urothelial) cancer, squamous cell cancer, or small cell prostate cancer. In some aspects, the prostate cancer is adenocarcinoma or acinar cell carcinoma. In some aspects, the method further comprises comprising imaging the biological sample. In some aspects, imaging comprising contacting the biological sample with one or more stains. In some aspects, the one or more stains comprises hematoxylin and eosin. In some aspects, the one or more stains comprise one or more optical labels. In some aspects, the one or more optical labels are selected from the group consisting of: fluorescent, radioactive, chemiluminescent, colorimetric labels, and combination thereof. In some aspects, the method further comprises identifying one or more cancerous regions in the biological sample using one or more stains. In some aspects, the biological sample is a tissue section or biopsy. In some aspects, the biological sample is a formalin-fixed, paraffin-embedded (FFPE) tissue sample, a frozen tissue sample, or a fresh tissue sample. In some aspects, the sample is a formalin-fixed, paraffin-embedded (FFPE) tissue sample. In some aspects, the biological sample comprises serial tissue sections or serial biopsies. In some aspects, the two or more analytes are protein analytes, or mRNA analytes, or a combination thereof. In some aspects, the expression level is a modulated expression level, an elevated expression level, or a decreased expression level compared to a reference expression level.

Moreover, the present disclosure generally relates to a system comprising: a. a storage element operable to store a dataset of a plurality of biological samples, wherein the dataset comprises, for each biological sample: analyte data for a plurality of analytes captured at a plurality of spatial locations from a biological sample; image data of the biological sample; and registration data of the imaged data, and wherein at least one biological sample is a reference sample and a second biological sample is a prostate tissue sample suspected of being cancerous; b. a computer program capable of linking the analyte data according to the spatial locations determined from a biological sample; and c. a processor operable to process the dataset through a machine learning module to determine the presence or absence of prostate cancer in the biological sample. In some aspects, the plurality of analytes comprise two or more analytes from one or more of (i), (ii), (iii), or (iv): (i) S100B, TGM4, SMOC1, LTF, ANPEP, SELE, PSCA, MT1G, ACTG2, WFDC2, SOCS3, PCP4, SYNM, GADD45B, TNC, GAS1, CNN1, ZNF185, DES, HSPB6, MSMB, MYLK, IGHG1, FLNC, ADAMTS4, MYL9, ADAM33, KRT5, PGM5, IGHV3-35, NEFH, JPH2, KRT15, TRIM29, SRD5A2, HSPB8, C11orf96, SPEG, ALDH2, FLNA, OLFM4, IGKV1D-12, TAGLN, TPM2, CRYAB, SMTN, KCNMB1, PMP22, ACTA2, and SYNPO2, or a fragment thereof; (ii) ADGRF1, OR51C1P, KCNC2, PLA2G7, SPON2, CAMK2N1, KCNN2, OR51E2, AGTR1, GRIN3A, KHDRBS3, KCNH8, ATP8A2, TMEM26, LUZP2, HPN, PLA2G2A, COL2A1, UNC80, DNASE2B, HOXC6, SPOCK1, AKR7A3, LRRN1, GOLM1, GJB1, FGFRL1, GHR, ACSM3, MCCC2, TRGV9, ZNF385B, CYP39A1, SEC11C, H2AFJ, MYO6, GPR160, BMPR1B, MBOAT2, GALNT3, CPNE4, ACER3, PLEKHH1, C9orf152, and PAQR6, or a fragment thereof; (iii) OR51E1, FGFR3, SPON2, KCNG3, OR51C1P, ARG2, DEGS1, OR51E2, TRGJP2, CPNE4, GOLM1, RDH11, ACSM1, PLEKHH1, SLC30A4, TRGC1, GDF11, KHDRBS3, SPOCK1, GDF15, FGFRL1, TRGV9, LRRN1, PLA2G7, TMSB15A, KCNN2, MARC1, PTPRT, C5orf30, COLEC12, HPN, MYO6, ADGRF1, UBE2E3, HGD, NSD2, KIF21A, HIST1H2AC, LETM1, COL9A2, BEND4, GJB1, ARFGEF3, PRAC1, F3, and SAMD5, or a fragment thereof; or (iv) IGHA1, JCHAIN, CD79A, IGHJ6, IGKC, SAA2, CCL19, IGLC1, SAA1, IGHG2, POU2AF1, DERL3, S100A9, IGHG1, LTB, C3, MZB1, IGKV4-1, RARRES1, LTF, IGHG3, IGHJ2, IGLV3-1, LYZ, LSP1, OLFM4, FCGBP, CFB, PDZK1IP1, KRT7, CORO1A, IL7R, PRDM1, CYBA, IGHM, LCN2, SERPINF1, HCLS1, CD53, PTPRC, PTGDS, NNMT, IGFBP4, WFDC2, CP, ITGB2, C1R, CLU, KRT15, and FHL2, or a fragment thereof. In some aspects, the plurality of analytes comprises two or more of CD24, KRT8, KRT18, or combinations thereof. In some aspects, the plurality of analytes comprises two or more of CD3D, CD3E, CD4, CD8A, CD247; CD79A, CD79B, IGHA1, IGHG1, JCHAIN, IGKC, IGLC1, fragments thereof, or combinations thereof. In some aspects, the plurality of analytes comprises two or more of TP63, KRT5, KRT14, fragments thereof, or combinations thereof. In some aspects, the plurality of analytes comprises two or more of CD4, Foxp3, IL17RB, CTLA4, FANK1, HAVCR1, CD25, GITR, LAG-3, CD127, fragments thereof, or combinations thereof. In some aspects, the plurality of analytes comprises two or more of CD4, CD3D, S100A4, IL7R, IFNG, fragments thereof, or combinations thereof. In some aspects, the plurality of analytes comprises two or more of CD4, IL7R, ICOS, CTLA4, TNFRSF4, TNFRS18, fragments thereof, or combinations thereof. In some aspects, the plurality of analytes comprises two or more of CD4, CD3D, IL17A, GZMA, S100A4, fragments thereof, or combinations thereof. In some aspects, the plurality of analytes comprises two or more of CD8, CD3D, S100A4, IFNG, GZMB, GZMA, IL2RB, fragments thereof, or combinations thereof.

In some aspects, the methods described herein can comprise assaying serially obtained biological samples from the subject at a plurality of time points and can further comprise identifying the expression level of one or more analytes in the serially obtained biological samples from the subject. In some aspects, the identified expression levels of the one or more analytes of the serial tissue samples can be compared to one another. In some aspects, based on this comparison, a prostate cancer treatment can be administered based on the comparison, a prostate cancer treatment dosage can be adjusted based on the comparison, or a prostate cancer treatment can be adjusted or ceased based on the comparison. In some aspects, based on this comparison, a prophylactic prostate cancer treatment can be administered based on the comparison, a prophylactic prostate cancer treatment dosage can be adjusted based on the comparison, or a prophylactic prostate cancer treatment can be adjusted or ceased based on the comparison.

As discussed herein, the methods of the present disclosure provide for a more detailed and in-depth at a tissue biopsy that can identify information that is not available to a pathologist upon staining and microscopic evaluation, thereby providing in a more personalized and accurate diagnosis and treatment regimen for a patient. Moreover, as discussed herein, the methods described here can identify patterns of gene expression that a pathologist, using established protocols of H&E staining with microscopy, is unable to determine. Furthermore, the methods described herein can be used to identify cellular changes indicative of cancer, in this case invasive carcinoma in prostate tissue, based on changes in basal and luminal cell localization within a prostate tissue sample. Moreover, the methods described herein can be used to monitor, for example, treatment regimens of prostate cancers and the efficacy of therapeutics for an individual. Further, identifying patterns in immune cell response to cancer types can be used as a prognostic indicator for the presence or absence of prostate cancer.

As discussed herein, the disclosed methods provide a powerful tool for identifying the spatial location and abundance of known prostate cancer gene markers, such as, for example, AMACR and ERG. For instance, the gene expression analysis using the methods described herein can reveal whether or not a carcinoma is fully invasive or only partially invasive. Additionally, as described herein, combining histological techniques, such as, for example, H&E and/or IHC, with the high throughput nature and revelatory power of instantly disclosed methods, the present disclosure generally provides a wholistic solution to personalized medicine that has not been present in traditional pathologist-based analysis for the detection, diagnosis, prognosis, and therapeutic monitoring of prostate cancer. Indeed, as shown by the data of the instant Examples, spatial gene expression analysis described herein identified aspects of tissues, for example prostate cancer tissues, to a level of resolution that was not obtained by manual histological annotation by a pathologist.

As further presented in the instant Examples, analysis of the invasive carcinoma region gene expression profiles in the adenocarcinoma prostate tissue sample revealed a different gene expression profile as compared to the non-invasive regions. The analysis revealed that known prostate cancer marker genes were present in the cancerous samples, however, a myriad of additional overexpressed and/or underexpressed genes in the cancerous prostate samples were discovered using the above-described methods. The identified genes could be used to further differentiate, for example, invasive adenocarcinoma over non-invasive adenocarcinoma.

Furthermore, the methods described herein provide for the discovery of novel predictive and diagnostic tumor markers, e.g., prostate cancer markers, individually or in combinations, that can potentially be used to further drive drug discovery and monitor therapeutic regimens with specificity for different cell types and regional cancer landscape maps for each cancer patient, thereby advancing the science of personal medicine.

### 3. Spatial Analysis Methodologies and Compositions

Spatial analysis methodologies and compositions described herein can provide a vast amount of analyte and/or expression data for a variety of analytes within a biological sample, e.g., a prostate tissue sample, at high spatial resolution, while retaining native spatial context. Spatial analysis methods and compositions can include, e.g., the use of a capture probe including a spatial barcode (e.g., a nucleic acid sequence that provides information as to the location or position of an analyte within a cell or a tissue sample (e.g., mammalian cell or a mammalian tissue sample) and a capture domain that is capable of binding to an analyte (e.g., a protein and/or a nucleic acid) produced by and/or present in a cell. Spatial analysis methods and compositions can also include the use of a capture probe having a capture domain that captures an intermediate agent for indirect detection of an analyte. For example, the intermediate agent can include a nucleic acid sequence that serves as a proxy for a target analyte. Detection of the intermediate agent is therefore indicative of the target analyte in the cell or tissue sample.

In some aspects, the capture domain is designed to detect one or more specific analytes of interest. For example, a capture domain can be designed so that it comprises a sequence that is complementary or substantially complementary to one analyte of interest. Thus, the presence of a single analyte can be detected. Alternatively, the capture domain can be designed so that it comprises a sequence that is complementary or substantially complementary to a region of multiple related analytes. In some aspects, the multiple related analytes are analytes that function in the same or similar cellular pathways or that have conserved homology and/or function. The design of the capture probe can be determined based on the intent of the user and can be any sequence that can be used to detect an analyte of interest. In some aspects, the capture domain sequence can therefore be random, semi-random, defined or combinations thereof, depending on the target analyte(s) of interest.

Non-limiting aspects of spatial analysis methodologies and compositions are described in U.S. Pat. Nos. 10,774,374, 10,724,078, 10,480,022, 10,059,990, 10,041,949, 10,002,316, 9,879,313, 9,783,841, 9,727,810, 9,593,365, 8,951,726, 8,604,182, 7,709,198, U.S. Patent Application Publication Nos. 2020/239946, 2020/080136, 2020/0277663, 2020/024641, 2019/330617, 2019/264268, 2020/256867, 2020/224244, 2019/194709, 2019/161796, 2019/085383, 2019/055594, 2018/216161, 2018/051322, 2018/0245142, 2017/241911, 2017/089811, 2017/067096, 2017/029875, 2017/0016053, 2016/108458, 2015/000854, 2013/171621, WO 2018/091676, WO 2020/176788, Rodrigues et al., Science 363(6434):1463-1467, 2019; Lee et al., Nat. Protoc. 10(3):442-458, 2015; Trejo et al., PLoS ONE 14(2):e0212031, 2019; Chen et al., Science 348(6233):aaa6090, 2015; Gao et al., BMC Biol. 15:50, 2017; and Gupta et al., Nature Biotechnol. 36:1197-1202, 2018; the Visium Spatial Gene Expression Reagent Kits User Guide (e.g., Rev C, dated June 2020), and/or the Visium Spatial Tissue Optimization Reagent Kits User Guide (e.g., Rev C, dated July 2020), both of which are available at the 10× Genomics Support Documentation website, and can be used herein in any combination. Further non-limiting aspects of spatial analysis methodologies and compositions are described herein.

Some general terminology that may be used in this disclosure can be found in Section (I)(b) of WO 2020/176788 and/or U.S. Patent Application Publication No. 2020/0277663, and is further discussed in § 1 above. In general, a “barcode” can be a label, or identifier, that conveys or is capable of conveying information (e.g., information about an analyte in a sample, a bead, and/or a capture probe). A barcode can be part of an analyte, or independent of an analyte. A barcode can be attached to an analyte. A particular barcode can be unique relative to other barcodes. In general, an “analyte” can include any biological substance, structure, moiety, or component to be analyzed, and a “target” can be an analyte of interest.

Analytes can be broadly classified into one of two groups: nucleic acid analytes, and non-nucleic acid analytes. Examples of non-nucleic acid analytes include, but are not limited to, lipids, carbohydrates, peptides, proteins, glycoproteins (N-linked or O-linked), lipoproteins, phosphoproteins, specific phosphorylated or acetylated variants of proteins, amidation variants of proteins, hydroxylation variants of proteins, methylation variants of proteins, ubiquitylation variants of proteins, sulfation variants of proteins, viral proteins (e.g., viral capsid, viral envelope, viral coat, viral accessory, viral glycoproteins, viral spike, etc.), extracellular and intracellular proteins, antibodies, and antigen binding fragments. In some aspects, the analyte(s) can be localized to subcellular location(s), including, for example, organelles, e.g., mitochondria, Golgi apparatus, endoplasmic reticulum, chloroplasts, endocytic vesicles, exocytic vesicles, vacuoles, lysosomes, etc. In some aspects, analyte(s) can be peptides or proteins, including without limitation antibodies and enzymes. Additional examples of analytes can be found in Section (I)(c) of WO 2020/176788 and/or U.S. Patent Application Publication No. 2020/0277663. In some aspects, an analyte can be detected indirectly, such as through detection of an intermediate agent, for example, a ligation product or an analyte capture agent (e.g., an oligonucleotide-conjugated antibody), such as those described herein

In some aspects, a biological sample can be obtained from the subject for analysis using any of a variety of techniques including, but not limited to, biopsy, surgery, and laser capture microscopy (LCM), and generally includes cells and/or other biological material from the subject. In some aspects, a biological sample can be a tissue section. In some aspects, a biological sample can be a fixed and/or stained biological sample (e.g., a fixed and/or stained tissue section). Non-limiting examples of stains include histological stains (e.g., hematoxylin and/or eosin) and immunological stains (e.g., fluorescent stains). In some aspects, a biological sample (e.g., a fixed and/or stained biological sample) can be imaged. Biological samples are also described in Section (I)(d) of WO 2020/176788 and/or U.S. Patent Application Publication No. 2020/0277663.

In some aspects, a biological sample is permeabilized with one or more permeabilization reagents. For example, permeabilization of a biological sample can facilitate analyte capture. Exemplary permeabilization agents and conditions are described in Section (I)(d)(ii)(13) or the Exemplary Aspects Section of WO 2020/176788 and/or U.S. Patent Application Publication No. 2020/0277663.

Array-based spatial analysis methods involve the transfer of one or more analytes from a biological sample to an array of features on a substrate, where each feature is associated with a unique spatial location on the array. Subsequent analysis of the transferred analytes includes determining the identity of the analytes and the spatial location of the analytes within the biological sample. The spatial location of an analyte within the biological sample is determined based on the feature to which the analyte is bound (e.g., directly or indirectly) on the array, and the feature's relative spatial location within the array.

In some aspects, a capture probe can be a molecule capable of capturing (directly or indirectly) and/or labelling an analyte (e.g., an analyte of interest) in a biological sample. In some aspects, the capture probe is a nucleic acid or a polypeptide. In some aspects, the capture probe includes a barcode (e.g., a spatial barcode and/or a unique molecular identifier (UMI)) and a capture domain). In some aspects, a capture probe can include a cleavage domain and/or a functional domain (e.g., a primer-binding site, such as for next-generation sequencing (NGS)). See, e.g., Section (II)(b) (e.g., subsections (i)-(vi)) of WO 2020/176788 and/or U.S. Patent Application Publication No. 2020/0277663. Generation of capture probes can be achieved by any appropriate method, including those described in Section (II)(d)(ii) of WO 2020/176788 and/or U.S. Patent Application Publication No. 2020/0277663.

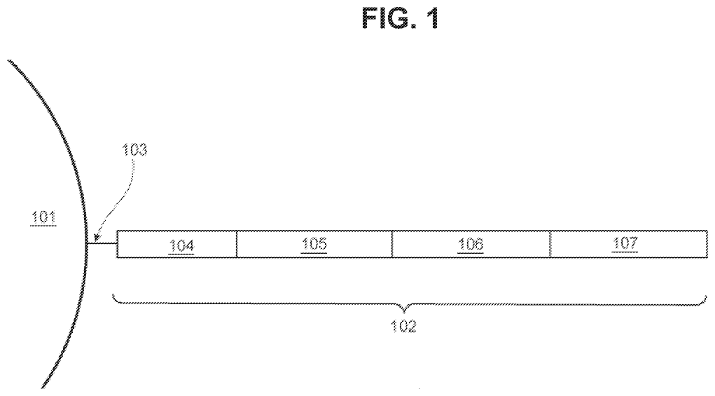

FIG. 1 is a schematic diagram showing an exemplary capture probe, as described herein. As shown, the capture probe 102 is optionally coupled to a feature 101 by a cleavage domain 103, such as a disulfide linker. The capture probe can include a functional sequence 104 that are useful for subsequent processing. The functional sequence 104 can include all or a part of sequencer specific flow cell attachment sequence (e.g., a P5 or P7 sequence), all or a part of a sequencing primer sequence, (e.g., a R1 primer binding site, a R2 primer binding site), or combinations thereof. The capture probe can also include a spatial barcode 105. The capture probe can also include a unique molecular identifier (UMI) sequence 106. While FIG. 1 shows the spatial barcode 105 as being located upstream (5′) of UMI sequence 106, it is to be understood that capture probes wherein UMI sequence 106 is located upstream (5′) of the spatial barcode 105 is also suitable for use in any of the methods described herein. The capture probe can also include a capture domain 107 to facilitate capture of a target analyte. In some aspects, the capture probe comprises one or more additional functional sequences that can be located, for example between the spatial barcode 105 and the UMI sequence 106, between the UMI sequence 106 and the capture domain 107, or following the capture domain 107. The capture domain can have a sequence complementary to a sequence of a nucleic acid analyte. The capture domain can have a sequence complementary to a connected probe described herein. The capture domain can have a sequence complementary to a capture handle sequence present in an analyte capture agent. The capture domain can have a sequence complementary to a splint oligonucleotide. Such splint oligonucleotide, in addition to having a sequence complementary to a capture domain of a capture probe, can have a sequence of a nucleic acid analyte, a sequence complementary to a portion of a connected probe described herein, and/or a capture handle sequence described herein.

The functional sequences can generally be selected for compatibility with any of a variety of different sequencing systems, e.g., Ion Torrent Proton or PGM, Illumina sequencing instruments, PacBio, Oxford Nanopore, etc., and the requirements thereof. In some aspects, functional sequences can be selected for compatibility with non-commercialized sequencing systems. Examples of such sequencing systems and techniques, for which suitable functional sequences can be used, include (but are not limited to) Ion Torrent Proton or PGM sequencing, Illumina sequencing, PacBio SMRT sequencing, and Oxford Nanopore sequencing. Further, in some aspects, functional sequences can be selected for compatibility with other sequencing systems, including non-commercialized sequencing systems.

In some aspects, the spatial barcode 105 and functional sequences 104 is common to all of the probes attached to a given feature. In some aspects, the UMI sequence 106 of a capture probe attached to a given feature is different from the UMI sequence of a different capture probe attached to the given feature.

In some aspects, more than one analyte type (e.g., nucleic acids and proteins) from a biological sample can be detected (e.g., simultaneously or sequentially) using any appropriate multiplexing technique, such as those described in Section (IV) of WO 2020/176788 and/or U.S. Patent Application Publication No. 2020/0277663.

In some aspects, detection of one or more analytes (e.g., protein analytes) can be performed using one or more analyte capture agents. In some aspects, an analyte capture agent can be an agent that interacts with an analyte (e.g., an analyte in a biological sample) and with a capture probe (e.g., a capture probe attached to a substrate or a feature) to identify the analyte. In some aspects, the analyte capture agent includes: (i) an analyte binding moiety (e.g., that binds to an analyte), for example, an antibody or antigen-binding fragment thereof; (ii) analyte binding moiety barcode; and (iii) a capture handle sequence. In some aspects, an analyte binding moiety barcode can be a barcode that is associated with or otherwise identifies the analyte binding moiety. In some aspects, an analyte capture sequence or capture handle sequence can be a region or moiety configured to hybridize to, bind to, couple to, or otherwise interact with a capture domain of a capture probe. In some aspects, a capture handle sequence is complementary to a capture domain of a capture probe. In some cases, an analyte binding moiety barcode (or portion thereof) may be able to be removed (e.g., cleaved) from the analyte capture agent.

In some aspects, a spatial barcode is associated with one or more neighboring cells, such that the spatial barcode identifies the one or more cells, and/or contents of the one or more cells, as associated with a particular spatial location. In some aspects, the association can occur by promoting analytes or analyte proxies (e.g., intermediate agents) out of a cell and towards a spatially-barcoded array (e.g., including spatially-barcoded capture probes). In some aspects, the association can occur by cleaving spatially-barcoded capture probes from an array and promoting the spatially-barcoded capture probes towards and/or into or onto the biological sample.

In some aspects, capture probes are configured to prime, replicate, and consequently yield optionally barcoded extension products from a template (e.g., a DNA or RNA template, such as an analyte or an intermediate agent (e.g., a ligation product or an analyte capture agent), or a portion thereof), or derivatives thereof (see, e.g., Section (II)(b)(vii) of WO 2020/176788 and/or U.S. Patent Application Publication No. 2020/0277663 regarding extended capture probes). In some aspects, capture probes are configured to form ligation products with a template (e.g., a DNA or RNA template, such as an analyte or an intermediate agent, or portion thereof), thereby creating ligations products that serve as proxies for a template.

In some aspects, an extended capture probe can be a capture probe having additional nucleotides added to the terminus (e.g., 3′ or 5′ end) of the capture probe thereby extending the overall length of the capture probe. For example, an “extended 3′ end” indicates additional nucleotides were added to the most 3′ nucleotide of the capture probe to extend the length of the capture probe, for example, by polymerization reactions used to extend nucleic acid molecules including templated polymerization catalyzed by a polymerase (e.g., a DNA polymerase or a reverse transcriptase). In some aspects, extending the capture probe includes adding to a 3′ end of a capture probe a nucleic acid sequence that is complementary to a nucleic acid sequence of an analyte or intermediate agent specifically bound to the capture domain of the capture probe. In some aspects, the capture probe is extended using reverse transcription. In some aspects, the capture probe is extended using one or more DNA polymerases. The extended capture probes include the sequence of the capture probe and the sequence of the spatial barcode of the capture probe.

In some aspects, extended capture probes are amplified (e.g., in bulk solution or on the array) to yield quantities that are sufficient for downstream analysis, e.g., via DNA sequencing. In some aspects, extended capture probes (e.g., DNA molecules) act as templates for an amplification reaction (e.g., a polymerase chain reaction).

Additional variants of spatial analysis methods, including in some aspects, an imaging step, are described in Section (II)(a) of WO 2020/176788 and/or U.S. Patent Application Publication No. 2020/0277663. Analysis of captured analytes (and/or intermediate agents or portions thereof), for example, including sample removal, extension of capture probes, sequencing (e.g., of a cleaved extended capture probe and/or a cDNA molecule complementary to an extended capture probe), sequencing on the array (e.g., using, for example, in situ hybridization or in situ ligation approaches), temporal analysis, and/or proximity capture, is described in Section (II)(g) of WO 2020/176788 and/or U.S. Patent Application Publication No. 2020/0277663. Some quality control measures are described in Section (II)(h) of WO 2020/176788 and/or U.S. Patent Application Publication No. 2020/0277663.

Spatial information can provide information of biological and/or medical importance. For example, the methods and compositions described herein can allow for: identification of one or more analytes, e.g., biomarkers (e.g., diagnostic, prognostic, and/or for determination of efficacy of a treatment) of a disease or disorder; identification of a candidate drug target for treatment of a disease or disorder; identification (e.g., diagnosis) of a subject as having a disease or disorder; identification of stage and/or prognosis of a disease or disorder in a subject; identification of a subject as having an increased likelihood of developing a disease or disorder; monitoring of progression of a disease or disorder in a subject; determination of efficacy of a treatment of a disease or disorder in a subject; identification of a patient subpopulation for which a treatment is effective for a disease or disorder; modification of a treatment of a subject with a disease or disorder; selection of a subject for participation in a clinical trial; and/or selection of a treatment for a subject with a disease or disorder.

Spatial information can provide information of biological importance. For example, the methods and compositions described herein can allow for: identification of transcriptome and/or proteome expression profiles (e.g., in healthy and/or diseased tissue); identification of multiple analyte types in close proximity (e.g., nearest neighbor analysis); determination of up- and/or down-regulated genes and/or proteins in diseased tissue; characterization of tumor microenvironments; characterization of tumor immune responses; characterization of cells types and their co-localization in tissue; and identification of genetic variants within tissues (e.g., based on gene and/or protein expression profiles associated with specific disease or disorder biomarkers).

Typically, for spatial array-based methods, a substrate functions as a support for direct or indirect attachment of capture probes to features of the array. In some aspects, a feature of the array can be an entity that acts as a support or repository for various molecular entities used in spatial analysis. In some aspects, some or all of the features in an array are functionalized for analyte capture. Exemplary substrates are described in Section (II)(c) of WO 2020/176788 and/or U.S. Patent Application Publication No. 2020/0277663. Exemplary features and geometric attributes of an array can be found in Sections (II)(d)(i), (II)(d)(iii), and (II)(d)(iv) of WO 2020/176788 and/or U.S. Patent Application Publication No. 2020/0277663.

Generally, analytes and/or intermediate agents (or portions thereof) can be captured when contacting a biological sample with a substrate including capture probes (e.g., a substrate with capture probes embedded, spotted, printed, fabricated on the substrate, or a substrate with features (e.g., beads, wells) comprising capture probes). In some aspects, the contact can be any contact (e.g., direct or indirect) such that capture probes can interact (e.g., bind covalently or non-covalently (e.g., hybridize)) with analytes from the biological sample. In some aspects, capture can be achieved actively (e.g., using electrophoresis), or passively (e.g., using diffusion). Analyte capture is further described in Section (II)(e) of WO 2020/176788 and/or U.S. Patent Application Publication No. 2020/0277663.

In some aspects, spatial analysis can be performed by attaching and/or introducing a molecule (e.g., a peptide, a lipid, or a nucleic acid molecule) having a barcode (e.g., a spatial barcode) to a biological sample (e.g., to a cell in a biological sample). In some aspects, a plurality of molecules (e.g., a plurality of nucleic acid molecules) having a plurality of barcodes (e.g., a plurality of spatial barcodes) are introduced to a biological sample (e.g., to a plurality of cells in a biological sample) for use in spatial analysis. In some aspects, after attaching and/or introducing a molecule having a barcode to a biological sample, the biological sample can be physically separated (e.g., dissociated) into single cells or cell groups for analysis. Some such methods of spatial analysis are described in Section (III) of WO 2020/176788 and/or U.S. Patent Application Publication No. 2020/0277663.

In some aspects, spatial analysis can be performed by detecting multiple oligonucleotides that hybridize to an analyte. In some aspects, for example, spatial analysis can be performed using RNA-templated ligation (RTL). Methods of RTL have been described previously. See, e.g., Credle et al., Nucleic Acids Res. 2017 Aug. 21; 45(14):e128. Typically, RTL includes hybridization of two oligonucleotides to adjacent sequences on an analyte (e.g., an RNA molecule, such as an mRNA molecule). In some aspects, the oligonucleotides are DNA molecules. In some aspects, one of the oligonucleotides includes at least two ribonucleic acid bases at the 3′ end and/or the other oligonucleotide includes a phosphorylated nucleotide at the 5′ end. In some aspects, one of the two oligonucleotides includes a capture domain (e.g., a poly(A) sequence, a non-homopolymeric sequence). After hybridization to the analyte, a ligase (e.g., SplintR ligase) ligates the two oligonucleotides together, creating a ligation product. In some aspects, the two oligonucleotides hybridize to sequences that are not adjacent to one another. For example, hybridization of the two oligonucleotides creates a gap between the hybridized oligonucleotides. In some aspects, a polymerase (e.g., a DNA polymerase) can extend one of the oligonucleotides prior to ligation. After ligation, the ligation product is released from the analyte. In some aspects, the ligation product is released using an endonuclease (e.g., RNAse H). The released ligation product serves as a proxy for the target analyte and can be captured by capture probes (e.g., instead of direct capture of an analyte) on an array, optionally amplified, and sequenced, thus determining the location and optionally the abundance of the analyte in the biological sample.

During analysis of spatial information, sequence information for a spatial barcode associated with an analyte is obtained, and the sequence information can be used to provide information about the spatial distribution of the analyte in the biological sample. Various methods can be used to obtain the spatial information. In some aspects, specific capture probes and the analytes they capture are associated with specific locations in an array of features on a substrate. For example, specific spatial barcodes can be associated with specific array locations prior to array fabrication, and the sequences of the spatial barcodes can be stored (e.g., in a database) along with specific array location information, so that each spatial barcode uniquely maps to a particular array location.

Alternatively, specific spatial barcodes can be deposited at predetermined locations in an array of features during fabrication such that at each location, only one type of spatial barcode is present so that spatial barcodes are uniquely associated with a single feature of the array. Where necessary, the arrays can be decoded using any of the methods described herein so that spatial barcodes are uniquely associated with array feature locations, and this mapping can be stored as described above.

When sequence information is obtained for capture probes and/or analytes during analysis of spatial information, the locations of the capture probes and/or analytes can be determined by referring to the stored information that uniquely associates each spatial barcode with an array feature location. In this manner, specific capture probes and captured analytes are associated with specific locations in the array of features. Each array feature location represents a position relative to a coordinate reference point (e.g., an array location, a fiducial marker) for the array. Accordingly, each feature location has an “address” or location in the coordinate space of the array.

Some exemplary spatial analysis workflows are described in the Exemplary Aspects section of WO 2020/176788 and/or U.S. Patent Application Publication No. 2020/0277663. See, for example, the Exemplary aspect starting with “In some non-limiting examples of the workflows described herein, the sample can be immersed . . . ” of WO 2020/176788 and/or U.S. Patent Application Publication No. 2020/0277663. See also, e.g., the Visium Spatial Gene Expression Reagent Kits User Guide (e.g., Rev C, dated June 2020), and/or the Visium Spatial Tissue Optimization Reagent Kits User Guide (e.g., Rev C, dated July 2020).

In some aspects, spatial analysis can be performed using dedicated hardware and/or software, such as any of the systems described in Sections (II)(e)(ii) and/or (V) of WO 2020/176788 and/or U.S. Patent Application Publication No. 2020/0277663, or any of one or more of the devices or methods described in Sections Control Slide for Imaging, Methods of Using Control Slides and Substrates for, Systems of Using Control Slides and Substrates for Imaging, and/or Sample and Array Alignment Devices and Methods, Informational labels of WO 2020/123320.

Suitable systems for performing spatial analysis can include components such as a chamber (e.g., a flow cell or sealable, fluid-tight chamber) for containing a biological sample. The biological sample can be mounted for example, in a biological sample holder. One or more fluid chambers can be connected to the chamber and/or the sample holder via fluid conduits, and fluids can be delivered into the chamber and/or sample holder via fluidic pumps, vacuum sources, or other devices coupled to the fluid conduits that create a pressure gradient to drive fluid flow. One or more valves can also be connected to fluid conduits to regulate the flow of reagents from reservoirs to the chamber and/or sample holder.

In some aspects, the systems can optionally include a control unit that includes one or more electronic processors, an input interface, an output interface (such as a display), and a storage unit (e.g., a solid state storage medium such as, but not limited to, a magnetic, optical, or other solid state, persistent, writeable and/or re-writeable storage medium). The control unit can optionally be connected to one or more remote devices via a network. The control unit (and components thereof) can generally perform any of the steps and functions described herein. Where the system is connected to a remote device, the remote device (or devices) can perform any of the steps or features described herein. The systems can optionally include one or more detectors (e.g., CCD, CMOS) used to capture images. The systems can also optionally include one or more light sources (e.g., LED-based, diode-based, lasers) for illuminating a sample, a substrate with features, analytes from a biological sample captured on a substrate, and various control and calibration media.

In some aspects, the systems can optionally include software instructions encoded and/or implemented in one or more of tangible storage media and hardware components such as application specific integrated circuits. The software instructions, when executed by a control unit (and in particular, an electronic processor) or an integrated circuit, can cause the control unit, integrated circuit, or other component executing the software instructions to perform any of the method steps or functions described herein.

In some aspects, the systems described herein can detect (e.g., register an image) the biological sample on the array. Some examples of methods to detect the biological sample on an array are described in PCT Application No. 2020/061064 and/or U.S. Patent Application Ser. No. 16/951,854.

Prior to transferring analytes from the biological sample to the array of features on the substrate, the biological sample can be aligned with the array. Alignment of a biological sample and an array of features including capture probes can facilitate spatial analysis, which can be used to detect differences in analyte presence and/or level within different positions in the biological sample, for example, to generate a three-dimensional map of the analyte presence and/or level. Some examples of methods to generate a two- and/or three-dimensional map of the analyte presence and/or level are described in PCT Application No. 2020/053655 and spatial analysis methods are generally described in WO 2020/061108 and/or U.S. patent application Ser. No. 16/951,864.

In some aspects, a map of analyte presence and/or level can be aligned to an image of a biological sample using one or more fiducial markers, e.g., objects placed in the field of view of an imaging system which appear in the image produced, as described in the Substrate Attributes Section, Control Slide for Imaging Section of WO 2020/123320, PCT Application No. 2020/061066, and/or U.S. Patent application Ser. No. 16/951,843. Fiducial markers can be used as a point of reference or measurement scale for alignment (e.g., to align a sample and an array, to align two substrates, to determine a location of a sample or array on a substrate relative to a fiducial marker) and/or for quantitative measurements of sizes and/or distances.

Additional spatial processes are described in PCT Patent Application Publication No. WO 2020/123320, which is incorporated by reference in its entirety.

### 4. Spatial Analysis in Prostate Cancer

a. Prostate Cancer

As described herein, the prostate cancer can be any type, form, or stage of prostate cancer. For instance, in some aspects, the prostate cancer can be adenocarcinoma, acinar cell carcinoma, ductal adenocarcinoma, transitional cell (or urothelial) cancer, squamous cell cancer, or small cell prostate cancer. In some aspects, the prostate cancer can be prostate cancer of any stage. In some aspects, the prostate cancer is a stage I prostate cancer. In some aspects, the prostate cancer is a stage IIA, stage BIB, or stage IIC prostate cancer. In some aspects, the prostate cancer is a stage IIIA, stage IIIB, or stage IIIC prostate cancer. In some aspects, the prostate cancer is a stage IVA or stage IVB prostate cancer.

In some aspects, a biological sample from a patient suspected of having prostate cancer has been previously analyzed, evaluated, and/or diagnosed, e.g., by pathological analysis. In some aspects, the pathological analysis can include staining, e.g., H&E staining or immunofluorescence staining. In some aspects, a pathologist has assigned a “Gleason score” to a biological sample that is a prostate tissue sample. In some aspects, the Gleason score of the tissue is 6 or lower. In some aspects, the Gleason score of the tissue is 7 or higher. In some aspects, the subject has previously undergone a PSA test and/or a DRE. In some aspects, the subject has previously had a prostate tissue sample obtained in the form of a biopsy, which was subjected to evaluation for the potential presence and/or severity of prostate cancer. In some aspects, a biological sample from a patient suspected of having prostate cancer prostate cancer can be imaged as a part of sample analysis. In some aspects, the sample can be contacted with one or more stains, e.g., hematoxylin and eosin or immunofluorescence. In some aspects, the one or more stains comprise one or more optical labels. In some aspects, the one or more optical labels are selected from the group consisting of: fluorescent, radioactive, chemiluminescent, calorimetric, colorimetric labels, and combinations of one or more of the foregoing. In some aspects, identifying one or more cancerous regions in the biological sample is performed using one or more stains specific to a cancer marker. In some aspects, the cancer marker is selected from the group consisting of pancytokeratin (pan-CK), CD45, SCGB2A1, MKI67, BRCA1, BRCA2, NPY, TMEFF2, PIK3CD, AMACR, ERG, KLK3, CRISP3, and combinations of one or more of the foregoing.
b. Prostate Cancer Analytes

The present disclosure includes methods of detecting one or more analytes, e.g., biomarkers indicative of prostate cancer, or indicative of an increased likelihood of developing prostate cancer, in various locations in a sample. Furthermore, the present disclosure includes methods of determining the expression level of one or more analytes, e.g., biomarkers indicative of prostate cancer, or indicative of an increased likelihood of developing prostate cancer, identifying the expression level of the one or more analytes, and correlating the identified expression level to a stage of prostate cancer or to an increased likelihood of developing prostate cancer. In some aspects, the detected analytes can be used, for instance, in methods for identifying candidate drug targets for treatment of a prostate cancer, identifying a candidate biomarker for efficacy of treatment of a prostate cancer, diagnosing a prostate cancer in a subject, identifying a subject with increased likelihood of developing prostate cancer, monitoring the progression of prostate cancer in a subject, determining the efficacy of a treatment for prostate cancer, identifying a patient subpopulation for which a therapeutic treatment is effective for prostate cancer, and modifying treatment for prostate cancer for a subject. The disclosed methods address gene expression at a spatial level, such that the location of gene expression in a sample is also realized by practicing these methods. The spatial pattern of expression of one or more prostate cancer related genes or immune cell genes can provide insight into the disease specific to an individual from which the sample originates, thereby advancing the field of personalized medicine for prostate cancer. Also provided herein are kits comprising antibodies to the specific candidate analytes, e.g., biomarkers identified herein as indicative of prostate cancer, or indicative of an increased likelihood of developing prostate cancer.

In some aspects, the analytes, e.g., biomarkers, indicative of prostate cancer, or indicative of an increased likelihood of developing prostate cancer identified herein that can be used for diagnostic, prognostic and therapeutic purposes include but are not limited to one or more, two or more, three or more, four or more, five or more, or six or more of S100B, TGM4, SMOC1, LTF, ANPEP, SELE, PSCA, MT1G, ACTG2, WFDC2, SOCS3, PCP4, SYNM, GADD45B, TNC, GAS1, CNN1, ZNF185, DES, HSPB6, MSMB, MYLK, IGHG1, FLNC, ADAMTS4, MYL9, ADAM33, KRT5, PGM5, IGHV3-35, NEFH, JPH2, KRT15, TRIM29, SRD5A2, HSPB8, C11orf96, SPEG, ALDH2, FLNA, OLFM4, IGKV1D-12, TAGLN, TPM2, CRYAB, SMTN, KCNMB1, PMP22, ACTA2, and SYNPO2, or a fragment thereof. In some aspects, the analytes, e.g., biomarkers, indicative of prostate cancer, or indicative of an increased likelihood of developing prostate cancer identified herein that can be used for diagnostic, prognostic and therapeutic purposes include but are not limited to one or more, two or more, three or more, four or more, five or more, or six or more of ADGRF1, TMEFF2, CRISP3, OR51C1P, KCNC2, PLA2G7, SPON2, CAMK2N1, NPY, KCNN2, OR51E2, AGTR1, GRIN3A, AMACR, KHDRBS3, KCNH8, ATP8A2, TMEM26, LUZP2, HPN, PLA2G2A, COL2A1, UNC80, DNASE2B, ERG, HOXC6, SPOCK1, AKR7A3, LRRN1, GOLM1, GJB1, FGFRL1, GHR, ACSM3, MCCC2, TRGV9, ZNF385B, CYP39A1, SEC11C, H2AFJ, MYO6, GPR160, BMPR1B, MBOAT2, GALNT3, CPNE4, ACER3, PLEKHH1, C9orf152, and PAQR6, or a fragment thereof. In some aspects, the analytes, e.g., biomarkers, indicative of prostate cancer, or indicative of an increased likelihood of developing prostate cancer identified herein that can be used for diagnostic, prognostic and therapeutic purposes include but are not limited to one or more, two or more, three or more, four or more, five or more, or six or more of NPY, TMEFF2, OR51E1, FGFR3, SPON2, KCNG3, CRISP3, OR51C1P, ARG2, DEGS1, OR51E2, TRGJP2, CPNE4, GOLM1, RDH11, ACSM1, PLEKHH1, SLC30A4, TRGC1, GDF11, KHDRBS3, SPOCK1, GDF15, FGFRL1, TRGV9, LRRN1, PLA2G7, TMSB15A, KCNN2, KLK3, MARC1, PTPRT, C5orf30, COLEC12, HPN, MYO6, ADGRF1, UBE2E3, HGD, NSD2, KIF21A, HIST1H2AC, LETM1, COL9A2, BEND4, GJB1, ARFGEF3, PRAC1, F3, and SAMD5, or a fragment thereof. In some aspects, the analytes, e.g., biomarkers, indicative of prostate cancer, or indicative of an increased likelihood of developing prostate cancer identified herein that can be used for diagnostic, prognostic and therapeutic purposes include but are not limited to one or more, two or more, three or more, four or more, five or more, or six or more of IGHA1, JCHAIN, CD79A, IGHJ6, IGKC, SAA2, CCL19, IGLC1, SAA1, IGHG2, POU2AF1, DERL3, S100A9, IGHG1, LTB, C3, MZB1, IGKV4-1, RARRES1, LTF, IGHG3, IGHJ2, IGLV3-1, LYZ, LSP1, OLFM4, FCGBP, CFB, PDZK1IP1, KRT7, CORO1A, IL7R, PRDM1, CYBA, IGHM, LCN2, SERPINF1, HCLS1, CD53, PTPRC, PTGDS, NNMT, IGFBP4, WFDC2, CP, ITGB2, C1R, CLU, KRT15, and FHL2, or a fragment thereof.

In some aspects, a biological sample, e.g., a prostate tissue sample, is examined for localized expression of one or more analytes. In some aspects, a prostate tissue sample can comprise a non-cancerous region, a pre-cancerous region, a cancerous region, or combinations thereof. In some aspects, the prostate tissue sample comprises adenocarcinoma, and the one or more, two or more, three or more, four or more, five or more, or six or more analytes include but are not limited to S100B, TGM4, SMOC1, LTF, ANPEP, SELE, PSCA, MT1G, ACTG2, WFDC2, SOCS3, PCP4, SYNM, GADD45B, TNC, GAS1, CNN1, ZNF185, DES, HSPB6, MSMB, MYLK, IGHG1, FLNC, ADAMTS4, MYL9, ADAM33, KRT5, PGM5, IGHV3-35, NEFH, JPH2, KRT15, TRIM29, SRD5A2, HSPB8, Cl lorf96, SPEG, ALDH2, FLNA, OLFM4, IGKV1D-12, TAGLN, TPM2, CRYAB, SMTN, KCNMB1, PMP22, ACTA2, and SYNPO2, or a fragment thereof. In some aspects, the prostate tissue sample comprises adenocarcinoma, and the one or more, two or more, three or more, four or more, five or more, or six or more analytes include but are not limited to ADGRF1, TMEFF2, CRISP3, OR51C1P, KCNC2, PLA2G7, SPON2, CAMK2N1, NPY, KCNN2, OR51E2, AGTR1, GRIN3A, AMACR, KHDRBS3, KCNH8, ATP8A2, TMEM26, LUZP2, HPN, PLA2G2A, COL2A1, UNC80, DNASE2B, ERG, HOXC6, SPOCK1, AKR7A3, LRRN1, GOLM1, GJB1, FGFRL1, GHR, ACSM3, MCCC2, TRGV9, ZNF385B, CYP39A1, SEC11C, H2AFJ, MYO6, GPR160, BMPR1B, MBOAT2, GALNT3, CPNE4, ACER3, PLEKHH1, C9orf152, and PAQR6, or a fragment thereof.

In some aspects, the prostate tissue sample comprises acinar cell carcinoma, and the one or more, two or more, three or more, four or more, five or more, or six or more analytes include but are not limited to NPY, TMEFF2, OR51E1, FGFR3, SPON2, KCNG3, CRISP3, OR51C1P, ARG2, DEGS1, OR51E2, TRGJP2, CPNE4, GOLM1, RDH11, ACSM1, PLEKHH1, SLC30A4, TRGC1, GDF11, KHDRBS3, SPOCK1, GDF15, FGFRL1, TRGV9, LRRN1, PLA2G7, TMSB15A, KCNN2, KLK3, MARC1, PTPRT, C5orf30, COLEC12, HPN, MYO6, ADGRF1, UBE2E3, HGD, NSD2, KIF21A, HIST1H2AC, LETM1, COL9A2, BEND4, GJB1, ARFGEF3, PRAC1, F3, and SAMD5, or a fragment thereof. In some aspects, the prostate tissue sample comprises acinar cell carcinoma, and the one or more, two or more, three or more, four or more, five or more, or six or more analytes include but are not limited to IGHA1, JCHAIN, CD79A, IGHJ6, IGKC, SAA2, CCL19, IGLC1, SAA1, IGHG2, POU2AF1, DERL3, S100A9, IGHG1, LTB, C3, MZB1, IGKV4-1, RARRES1, LTF, IGHG3, IGHJ2, IGLV3-1, LYZ, LSP1, OLFM4, FCGBP, CFB, PDZK1IP1, KRT7, CORO1A, IL7R, PRDM1, CYBA, IGHM, LCN2, SERPINF1, HCLS1, CD53, PTPRC, PTGDS, NNMT, IGFBP4, WFDC2, CP, ITGB2, C1R, CLU, KRT15, and FHL2, or a fragment thereof.

In some aspects of any of the methods described herein, the expression level or abundance of an analyte can be dysregulated. A dysregulated expression level can be an elevated expression level, a decreased expression level, or an otherwise modulated expression level as compared to a reference expression level. In some aspects, the reference level is the entire tissue, particularly in aspects where a region of interest is analyzed. In some aspects, the reference level is another biological sample.

c. Subjects

A subject can be any appropriate subject, e.g., any appropriate animal. In some aspects, a subject can be a mammal. In some aspects, a mammal can be a mouse. In some aspects, a mammal can be a rat. In some aspects, a mammal can be a nonhuman primate, such as a chimpanzee, a gorilla, an orangutan, a rhesus monkey, a cynomolgus monkey, a Taiwanese macaque, a green monkey, a squirrel monkey, tamarin, a marmoset, or a mouse lemur. In some aspects, a mammal can be a human. In some aspects, a subject can be an animal model of prostate cancer. In some aspects, a mammal can be a mammalian model of prostate cancer. In some aspects, an animal model of prostate cancer can express one or more human genes.

d. Spatial Cell-Based Analytical Methodology and Methods Involving Sorting Subsets of Nucleic Acids

Further provided herein are methods for sorting subsets of nucleic acids from a biological sample, e.g., a prostate tissue sample, into a cluster. For example, in some aspects, such methods include contacting the biological sample with a plurality of capture probes, wherein a capture probe comprises a capture domain and a spatial barcode having a sequence; releasing nucleic acids from the biological sample, wherein members of the released nucleic acids are specifically bound by the capture domain(s); determining, for the nucleic acids that are specifically bound by the capture domain(s), (1) all or a portion of a sequence of the spatial barcode, or a complement thereof, and (2) all or a portion of a sequence of the nucleic acid or a complement thereof, and using the determined sequences of (1) and (2) to identify the location and amount of the nucleic acids in the biological sample; and comparing the determined location and amount of the nucleic acids at a plurality of different locations in the biological sample.

In some aspects, methods of differentiating cell types in a biological sample are provided herein, e.g., the methods comprise sorting a subset of nucleic acids into a cluster based on the determined location and amount of the nucleic acids at the plurality of different locations in the biological sample, and using the cluster(s) to differentiate cell types in the biological sample, such as differentiating basal cells and luminal cells. In some aspects, methods of identifying a biological sample are provided herein, e.g., the methods comprise sorting a subset of nucleic acids into a cluster based on the determined location and amount of the nucleic acids at the plurality of different locations in the biological sample, and using the cluster(s) to identify the biological sample (e.g., the type of tissue the biological sample is from). In some aspects, methods of generating an image of a biological sample are provided herein, e.g., the methods comprise sorting a subset of nucleic acids into a cluster based on the determined location and amount of the nucleic acids at the plurality of different locations in the biological sample, and using the cluster(s) to generate an image of the biological sample.

In some aspects, the methods described herein provide for analyzing molecular heterogeneity in a biological sample, e.g., sorting a subset of nucleic acids into a cluster based on the determined location and amount of the nucleic acids at the plurality of different locations in the biological sample, and using the cluster(s) to identify molecular heterogeneity in the biological sample relative to a reference biological sample. In some aspects, the methods described herein provide for identifying a subject as having a dysregulated gene expression in a biological sample obtained from said subject, e.g., sorting a subset of nucleic acids into a cluster based on the determined location and amount of the nucleic acids at a plurality of different locations in the biological sample, and using the cluster(s) to identify at least one region in the biological sample with dysregulated gene expression relative to a reference biological sample. In some aspects, the methods described herein provide for identifying a subject as having a cellular anomaly, e.g., sorting a subset of nucleic acids into a cluster based on the determined location and amount of the nucleic acids at the plurality of different locations in a biological sample obtained from the subject, and using the cluster(s) to identify at least one cellular anomaly in the biological sample. In some aspects, the methods described herein provide for assessing the efficacy of a treatment or therapy in a subject, e.g., sorting a subset of nucleic acids into a cluster based on the determined location and amount of the nucleic acids at the plurality of different locations in a biological sample obtained from the subject, and using the cluster(s) to identify at least one region in the biological sample having restored gene expression. In some aspects, the methods described herein provide for determining the heterogeneity of a sample, e.g., to determine the spatial or locational distribution and spatial density of one or more analytes, e.g., one or more biomarkers. For example, in some aspects, methods described herein can determine that even though the net level of one or more analytes, e.g., one or more biomarkers, disclosed herein can be relatively the same between a normal and diseased tissue, the density or localization of that biomarker within the tissue can vary. Thus, in some aspects, methods described herein can provide for determination that an analyte can be expressed at one level in one area of a sample and can be expressed at a second level at a second location in the sample. In some aspects, the expression level can be the same, or the expression level can be increased, decreased, or otherwise modulated. In some aspects, the expression of the one or more analytes can be correlated with an image of the tissue. In some aspects, the amount of one or more analytes identified falls outside a predetermined threshold amount. In some aspects, the amount of one or more analytes identified is elevated compared to the amount of a reference analyte. In some aspects, the amount of one or more analytes identified is reduced compared to the amount of a reference analyte.

In some aspects, methods described herein provide for comparing at least two biological samples are provided herein, e.g., the methods comprise sorting a subset of nucleic acids into a first set of clusters based on the determined location and amount of the nucleic acid sat the plurality of different locations in a first biological sample; sorting a subset of nucleic acids into a second set of clusters based on the determined location and amount of the nucleic acids at the plurality of different locations in a second biological sample; and comparing the first and second sets of clusters (i.e., the clusters from the first and second biological samples).

In some aspects, the first biological sample is from the same subject as the second biological sample. In some aspects, there is a period of time between acquiring the first biological sample and acquiring the second biological or subsequent samples from the subject. In some aspects, the period of time is about 1 day to about five years, e.g., about 1 day to about 10 days, about 1 day to about 1 month, about 1 day to about 6 months, about 1 day to about 1 year, about 1 day to about 1.5 years, about 1 day to about 2 years, about 1 day to about 2 years, about 1 day to about 4 years, about 4 years to about 5 years, about 3 years to about 5 years, about 2 years to about 5 years, or about 1 year to about 5 years. For example, about 1.5 years to about 2 years, about 1 year to about 2 years, about 6 months to about 2 years, about 1 to about 3 years, or about 2 to about 4 years. In some aspects, the period of time is about 1 month, about 6 months, about 1 year, about 2 years, about 3 years, about 4 years, or about 5 years. In some aspects, the method further comprises comparing the clusters from additional biological samples obtained from the subject before and after the period of time.

In some aspects, the first biological sample is obtained from a first subject and the second biological sample is obtained from a second subject. In some aspects, the second biological sample is obtained from a healthy subject. In some aspects, the first biological sample is obtained from a subject at risk (e.g., increased risk) of developing a disease, e.g., prostate cancer.

In some aspects, methods provided herein include sorting a subset of nucleic acids into a first set of clusters based on the determined amount and location of the nucleic acids at the plurality of different locations in the biological sample; and comparing the set of clusters to a reference set of clusters. In some aspects, the reference set of clusters is a normalized set of clusters from more than one reference biological sample. In some aspects, each of the more than one reference biological sample comprises the same type of tissue as the biological sample obtained from the subject.

In some aspects, a method as described herein can further comprise identifying a subpopulation of cells in the biological sample. In some aspects, the biological sample is obtained from a biopsy. In some aspects, the biological sample is obtained from a surgical excision. In some aspects, the biological sample is obtained by venipuncture. Methods of processing biological samples (e.g., prostate tissue from a biopsy) for use in the methods described herein are well-known in the art.

e. Locations in Sample

As used herein, a location in a sample can be any appropriate location. For instance, a location in a prostate tissue sample can be any location in the prostate of a subject. For example, the prostate tissue sample location can include any form of prostate tissue, such as glandular and/or fibrous tissue. In some aspects, the prostate tissue sample location can comprise epithelial cells and/or stromal cells. In some aspects, the prostate tissue sample location can comprise luminal cells, basal cells, neuroendocrine cells, smooth muscle cells, fibroblasts, or combinations of any one or more of the foregoing. In some aspects, a prostate tissue sample is obtained by biopsy, such as a needle biopsy or other method for obtaining a tissue sample from a subject, at any location of a prostate of a subject. A biopsied tissue sample can be sectioned, for example via microtome sectioning into smaller tissue slices from a block sample, for example sections of around 5-20 μm thickness can be sued in the spatial methods described herein.

f. Reference Levels or Reference Amounts

In some aspects, the methods described herein comprise determining a reference level (or amount) of an analyte, e.g., a biomarker. In some aspects, a reference level of a biomarker can be determined based on a level of the biomarker in a corresponding sample (e.g., a prostate tissue sample of a control subject, e.g., a control subject not diagnosed, not presenting with any of the symptoms of prostate cancer, not having a family history of prostate cancer, and not having any known risk factors of prostate cancer) at a corresponding position. In some aspects, a reference level of a biomarker can be determined based on an amount of the biomarker in one or more other locations in a sample. A reference level can be obtained from a prostate tissue sample from a subject that is not the same subject providing a prostate tissue sample that may be cancerous. Alternatively, a reference level can be obtained from a prostate tissue sample from the same subject that is providing a prostate tissue sample that may be cancerous, wherein the prostate tissue sample used as a reference is taken from a location in the subject where prostate cancer is not suspected as occurring, thereby providing a matched normal tissue sample from the same subject.

In some aspects, a reference level can be based on a reference level as published by an appropriate body (e.g., a government agency (e.g., the United States Food and Drug Administration) or a professional organization (e.g., the American Medical Association or American Psychiatric Association)), for example, a reference level that is a threshold level for an analyte, e.g., a biomarker, at the location in the prostate of an animal.

In some aspects, a reference level of an analyte, e.g., biomarker, can be determined based on any appropriate criteria. For example, in some aspects, a reference level of an analyte can come from an age-matched healthy subject. In some aspects, a reference level of an analyte can come from a sex-matched healthy subject or a sex-matched healthy subject population. In some aspects, a reference level of an analyte can come from an age-matched, sex-matched healthy subject or an age-matched, sex-matched healthy subject population. In some aspects, a reference level of an analyte can come from an aggregate sample (e.g., an average of 2 or more individual) of healthy subjects (e.g., that are age-matched and/or sex-matched). In some aspects, a reference level of an analyte can come from a sample (biopsy, etc.) that was historically taken from a subject when that subject was deemed healthy or taken from a subject prior to an associated treatment. In some aspects, a reference level of analyte is a baseline level taken from a subject taken prior to a treatment.

A healthy subject can be any appropriate healthy subject. In some aspects, a healthy subject has one or more of: no known prostate cancer, presentation of no symptoms of prostate cancer, no known genetic mutations associated with risk of a prostate cancer, no family medical history of prostate cancer, and no behavioral risk factors of prostate cancer.

In some cases, a level (e.g., abundance) of an analyte, e.g., a biomarker, can be dysregulated, such as increased, decreased, or otherwise modulated, relative to a reference level. In some cases, a level (e.g., abundance) of an analyte, e.g., biomarker, can be elevated relative to a reference level. For example, a level of a biomarker can be at least 0.2-fold (e.g., at least 0.4-fold, at least 0.6-fold, at least 0.8-fold, at least 1-fold, at least 1.3-fold, 1.5-fold, 2-fold, 3-fold, 4-fold, 5-fold, 6-fold, 7-fold, 8-fold, 9-fold, 10-fold, 12-fold, 15-fold, 18-fold, 20-fold, 25-fold, 30-fold, 40-fold, 50-fold, or more) greater than a reference level (e.g., any of the exemplary reference levels described herein or known in the art).

In some cases, a level of an analyte, e.g., a biomarker, can be decreased relative to a reference level. For example, a level of a biomarker can be at least 5% less, at least 10% less, at least 15% less, at least 20% less, at least 25% less, at least 30% less, at least 35% less, at least 40% less, at least 45% less, at least 50% less, at least 55%, at least 60% less, at least 65% less, at least 70% less, at least 75% less, at least 80% less, at least 85% less, at least 90% less, at least 95% decreased (e.g., about a 5% to about a 99% decrease, about a 5% decrease to about a 80% decrease, about a 5% decrease to about a 60% decrease, about a 5% decrease to about a 40% decrease, about a 5% decrease to about a 20% decrease, about a 20% decrease to about a 95% decrease, about a 20% decrease to about a 80% decrease, about a 20% decrease to about a 60% decrease, about a 20% decrease to about a 40% decrease, about a 40% decrease to about a 99% decrease, about a 40% decrease to about a 80% decrease, about a 40% decrease to about a 60% decrease, about a 60% decrease to about a 99% decrease, about a 60% decrease to about a 80% decrease, about a 80% decrease to about a 99% decrease) as compared to a reference level (e.g., any of the exemplary reference levels described herein). Other suitable reference levels and methods of determining the same will be apparent to those skilled in the field.

g. Biomarkers and Candidate Biomarkers

As discussed herein, the one or more analytes can be one or more biomarkers. In some aspects, a biomarker can be a nucleic acid (e.g., genomic DNA (gDNA), mRNA, or rRNA (e.g., bacterial 16S rRNA)), a protein (e.g., an enzyme, a cell surface marker, a structural protein, a tumor suppressor, an antibody, a cytokine, a peptide hormone, or an identifiable fragment, precursor, or degradation product of any thereof), a lipoprotein, a fatty acid, a cell (e.g., a cell type, for example, in a location indicative of disease), or a small molecule (e.g., an enzymatic cofactor, a hormone (e.g., a steroid hormone or a eicosanoid hormone), or a metabolite). In some aspects, a biomarker can include an alteration in a nucleic acid (e.g., an insertion, a deletion, a point mutation, and/or methylation), for example, relative to a wildtype or control nucleic acid. In some aspects, a biomarker can include an alteration in a protein (e.g., an inserted amino acid, a deletion of an amino acid, an amino acid substitution, and/or a post-translational modification (e.g., presence, absence, or a change in, for example, acylation, isoprenylation, phosphorylation, glycosylation, methylation, hydroxylation, amidation, and/or ubiquitinylation)), for example, relative to a control or wildtype protein.

In some aspects, a biomarker is a nucleic acid. In some aspects, a biomarker is an mRNA molecule. In some aspects, a biomarker is a protein. In some aspects, a biomarker is an enzyme. In some aspects, a biomarker is a cell surface marker. In some aspects, biomarkers are identified because they co-localize with one or more additional biomarkers.

h. Clusters

As described herein, many methods can be used to help identify a cluster of analytes, e.g., biomarkers. Non-limiting examples of such methods include nonlinear dimensionality reduction methods such as t-distributed stochastic neighbor embedding (t-SNE), global t-distributed stochastic neighbor embedding (g-SNE), and uniform manifold approximation and projection (UMAP).

Any number of clusters can be identified. In some aspects, 2 to 500 clusters can be identified using the methods as described herein. For example, 2 to 10, 2 to 20, 2 to 50, 2 to 75, 2 to 100, 2 to 150, 2 to 200, 2 to 300, 2 to 400, 400 to 500, 300 to 500, 200 to 500, 100 to 500, 75 to 500, 50 to 500, or 25 to 200 clusters can be identified. In some aspects, 25 to 75, 50 to 100, 50 to 150, 75 to 150, or 100 to 200 clusters can be identified.

Any number of nucleic acids can be sorted into a cluster. For example, a cluster can include about 1 to about 200,000 nucleic acids. In some aspects, a cluster can include about 1 to about 150,000, about 1 to about 100,000, about 1 to about 75,000, about 1 to about 50,000, about 100,000 to about 200,000, or about 50,000 to about 200,000 nucleic acids. In some aspects, a cluster includes about 2 to about 25,000 nucleic acids. For example, about 2 to about 50, about 2 to about 100, about 2 to about 500, about 2 to about 1,000, about 2 to about 5,000, about 2 to about 10,000, about 2 to about 15,000, about 2 to about 20,000, about 20,000 to about 25,000, about 15,000 to about 25,000, about 10,000 to about 25,000, about 5,000 to about 25,000, about 1,000 to about 25,000, about 500 to about 25,000, or about 100 to about 25,000 nucleic acids.

In some aspects, a nucleic acid included in a cluster is different than each of the other nucleic acids in the cluster. For example, the nucleic acid has a sequence that is not identical to any of the other nucleic acids in the cluster. In some aspects, a nucleic acid corresponds to a gene.

i. Methods of Treatment/Identifying a Diagnostic or Prognostic Marker of Prostate Cancer or a Candidate Biomarker for Efficacy of a Treatment of Prostate Cancer

In some aspects, provided herein are methods for identifying a diagnostic or prognostic biomarker of prostate cancer, and determining a candidate biomarker for determining efficacy of a treatment of prostate cancer. A diagnostic or prognostic biomarker is an analyte (e.g., nucleic acid, protein) that can be used to identify and/or determine the presence of prostate cancer, or determine the likelihood that prostate cancer can or will be identified in a subject. In some aspects, a candidate prognostic biomarker is detected and used to predict the prognosis of a subject's prostate cancer. In some aspects, the diagnostic or prognostic biomarker is increased relative to a reference sample. In some aspects, the diagnostic or prognostic biomarker is decreased relative to a reference sample. In some aspects, the diagnostic or prognostic biomarker is modulated relative to a reference sample. In some aspects, the diagnostic or prognostic biomarker is dysregulated relative to a reference sample.

The methods can include (a) determining level(s) of one or more biomarker(s) in a location in a sample comprising prostate tissue obtained from an animal having prostate cancer; (b) identifying: (i) one or more biomarker(s) showing elevated level(s) in the location in the sample as compared to reference level(s) of the one or more biomarker(s) and/or (ii) one or more biomarker(s) showing decreased level(s) in the location in the sample as compared to reference level(s) of the one or more biomarker(s) as diagnostic, or prognostic biomarker(s) of the prostate cancer and/or or as candidate biomarker(s) for determining efficacy of a treatment of the prostate cancer. In some aspects, a reference level of the one or more biomarker(s) is a level of the one or more biomarker(s) in a corresponding location in a sample comprising prostate tissue obtained from a control animal. In some aspects, an animal can be any of the animals described herein. In some aspects, an animal can be a mammal.

In some aspects, the methods can include identifying one or more biomarker(s) showing elevated level(s) in the location in the sample as compared to reference level(s) of the one or more biomarker(s) as diagnostic, or prognostic biomarker(s) of the prostate cancer and/or or as candidate biomarker(s) for determining efficacy of a treatment of the prostate cancer. In some aspects, the methods can include identifying one or more biomarker(s) showing decreased level(s) in the location in the sample as compared to reference level(s) of the one or more biomarker(s) as diagnostic or prognostic biomarker(s) of the prostate cancer, and/or or as candidate biomarker(s) for determining efficacy of a treatment of the prostate cancer.

The identified diagnostic markers (identified using the methods described herein) can then be used in methods of diagnosing prostate cancer, such as a stage of prostate cancer, and comparing the level of the diagnostic marker to a reference level (e.g., any of the reference levels described in herein (e.g., a level of the biomarker in a similar biological sample from a control subject or a population of control subjects)).

For example, an increase in the level of the diagnostic biomarker (e.g., one or more of AMACR and ERG) in a biological sample obtained from a subject (for a diagnostic biomarker identified as having an increased level as compared to a reference level) as compared to a level of the biomarker in a similar biological sample from a control subject or a population of control subjects indicates that the subject has prostate cancer.

Such methods can be used to identify patients having an early stage of prostate cancer (e.g., before the presentation of symptoms). In such methods where a patient is diagnosed as having an early stage of prostate cancer (e.g., using any of the methods described herein), the subject can be administered a treatment recognized as appropriate for the stage of prostate cancer identified. In some aspects, the biomarker could be used as an indicator of progression or regression of the disorder. In some aspects, the biomarker can be used to monitor a treatment regimen in a subject to aide in determining the efficacy of a treatment for prostate cancer.

In some aspects, a biological sample is placed on a substrate that comprises a plurality of capture probes. After permeabilization of the biological sample, analytes (e.g., mRNA molecules) migrate and hybridize to the capture probe. In some aspects, the capture probe includes a capture domain that includes a poly-thymine (T) sequence that can indiscriminately hybridize to a poly(A) mRNA sequence of an analyte. Once the capture probes capture the analyte(s), first strand cDNA created by template switching and reverse transcriptase is then denatured and the second strand is then extended. The second strand cDNA is then denatured from the first strand cDNA, neutralized, and transferred to a tube. cDNA quantification and amplification can be performed using standard techniques discussed herein. The cDNA can then be subjected to library preparation and indexing, including fragmentation, end-repair, and a-tailing, and indexing PCR steps. The library preparation can optionally be quality controlled to verify the success of the library preparation methods. The cDNA fragments can then be sequenced using, for example, paired-end sequencing using TruSeq Read 1 and TruSeq Read 2 as sequencing primer sites.

In some aspects, arrays (e.g., glass slides) include a plurality of capture probes that bind to one or more specific biological targets in a sample (i.e., targeted analysis). In some aspects, the capture probes hybridize to specific analytes, e.g., under appropriate conditions where oligonucleotide capture probes can hybridize to the target nucleic acids in a sequence-specific manner. That is, the capture probe includes a sequence that is specific to an analyte of interest, and the capture probe discriminately captures the targeted analyte. In some aspects, analytes that do not hybridize to capture probes are removed (e.g., analytes that do not interact with capture domains of the capture probes). In some aspects, removal of analytes that did not interact with a capture probe can be accomplished by, e.g., washing the sample to remove such analytes.

In some aspects, targeted capture occurs through enrichment of targets of interest after analytes are non-discriminately captured by capture probes on an array. In some instances of this aspect, analytes (e.g., mRNA) is captured by a capture probe. In some aspects, the capture probe includes a sequence that hybridizes to an analyte. In some aspects, the capture probe includes poly-thymine (T) sequence that hybridizes to a poly(A) sequence of an mRNA analyte. After the analytes are captured by the capture probe, the analytes are pooled and amplified. In some aspects, after amplification, specific analytes of interest are enriched in the pool. In some aspects, a plurality of bait oligonucleotides are added to the pool. In some aspects, a bait oligonucleotide includes a capture domain that binds specifically to all or a portion of the sequence of the nucleic acid from the biological sample, or a complement thereof. In some aspects, the bait oligonucleotide includes a molecular tag. In some aspects, the molecular tag include a moiety such as a streptavidin molecule, an avidin molecule, a biotin molecule, or a fluorophore molecule. In some aspects, the moiety can be used to isolate bait oligonucleotides that have hybridized to a target sequence of interest. After isolation of the hybridized bait oligonucleotide/target, the target can be isolated, purified, and optionally amplified using methods known in the art. In some aspects, this enriched pool of a target of interest can then be sequenced to identify all or a portion of the sequence of the spatial barcode (from the initial capture probe) or the complement thereof, and all or a portion of the sequence of the nucleic acid from the biological sample, and using the determined sequences of (i) and (ii) to identify the location of the nucleic acid in the biological sample.

In some aspects, a plurality of bait oligonucleotides can be designed so that each bait oligonucleotide sequence theoretically hybridizes to a unique target of interest. In some aspects, the designed bait oligonucleotides are at least 40 nucleotides in length. In some aspects, the bait oligonucleotides are about 120 nucleotides in length. In some aspects, the bait oligonucleotides range from about 40 to about 160 nucleotides in length. In some aspects, a panel of bait oligonucleotides are used to target one analyte of interest or a plurality of analytes of interest. In some aspects, the plurality of analytes of interest is between five genes and twenty thousand genes. In some aspects, the plurality of analytes is between one hundred genes and ten thousand genes. In some aspects, the plurality of analytes is between five hundred analytes and two thousand analytes. In some aspects, the plurality of analytes is more than 10, more than 50, more than 100, more than 500, more than 1000, more than 2000, more than 5000, more than 10000, more than 15000, or more than 20000 analytes. It is appreciated that panels and bait oligonucleotides can be designed to target analytes of interest in a specific setting (e.g., for a specific tissue or for a specific pathological setting such as cancer). In some cases, spatial analysis can be performed by detecting multiple oligonucleotides that hybridize to one or more analytes. In some aspects, for example, spatial analysis can be performed using RNA-templated ligation (RTL). Methods for practicing spatial RNA templated ligation are described in WO2021/133849A1, incorporated herein by reference in its entirety. Briefly, RTL steps include hybridization of two oligonucleotides to adjacent sequences of an analyte (e.g., an RNA molecules, e.g., an mRNA molecule). In some aspects, the oligonucleotides are DNA molecules. In some aspects, one of the oligonucleotides includes at least two ribonucleic acid bases at the 3′ end and the other oligonucleotide includes a phosphorylated nucleotide at the 5′ end. In some aspects, one of the two oligonucleotides includes a capture probe binding domain (e.g., a poly(A) sequence).

After hybridization, a ligase (e.g., T4 DNA ligase) ligates the oligonucleotides together, creating a ligation product. In some aspects, the two oligonucleotides hybridize to sequences that are not adjacent to one another. For example, hybridization of the two oligonucleotides creates a gap between the hybridized oligonucleotides. In some aspects, a polymerase (e.g., a DNA polymerase) can extend one of the oligonucleotides prior to ligation. In some aspects, after ligation, the ligation product is released from the analyte. In some aspects, the ligation product is released using an endonuclease (e.g., RNAse H). The released ligation product can then be captured by capture probes on an array, amplified, and sequenced, thus determining the location and abundance of the analyte in the biological sample.

In some aspects, the methods include optimizing permeabilization of a biological sample. Optimizing permeabilization can be useful for identifying intracellular analytes. Permeabilization optimization can include selection of permeabilization agents, concentration of permeabilization agents, and permeabilization duration. In general, a biological sample can be permeabilized by exposing the sample to one or more permeabilizing agents. Suitable agents for this purpose include, but are not limited to, organic solvents (e.g., acetone, ethanol, and methanol), detergents (e.g., saponin, Triton X-100™, Tween-20™, or sodium dodecyl sulfate (SDS)), and enzymes (e.g., trypsin, proteases (e.g., proteinase K). In some aspects, the detergent is an anionic detergent (e.g., SDS or N-lauroylsarcosine sodium salt solution). In some aspects, the biological sample can be permeabilized during any of the steps described herein (e.g., using any of the detergents described herein, e.g., SDS and/or N-lauroylsarcosine sodium salt solution) before or after enzymatic treatment (e.g., treatment with any of the enzymes described herein, e.g., trypin, proteases (e.g., pepsin and/or proteinase K)).

At any point during the methods disclosed herein, the biological sample can be imaged. For example, a region of interest can be identified in a biological sample using a variety of different techniques, e.g., expansion microscopy, bright field microscopy, dark field microscopy, phase contrast microscopy, electron microscopy, fluorescence microscopy, reflection microscopy, interference microscopy, confocal microscopy, and visual identification (e.g., by eye), and combinations thereof.

In some aspects, this disclosure further provides devices for holding or supporting substrates for use in the methods disclosed herein. In particular, the devices include a first and second members that receive a first and second substrate, respectively. In some aspects, the devices of the disclosure can be used for sandwiching the first and second substrates together for spatial transcriptomics applications. In some aspects, the first substrate can support a sample (e.g., a biological substrate) on its surface. In some aspects, the second substrate can include a plurality of barcoded probes and/or permeabilization reagents. In some aspects, the biological sample is permeabilized to allow analytes to be released from the sample on the first substrate and bind (e.g., hybridize) to the capture probes attached to the second substrate. Methods and devices relating to substrates that are used in sandwiching methods are disclosed in WO 2020/123320, which in incorporated by reference in its entirety.

In some aspects, the methods can include performing an experiment to validate whether the one or more identified candidate prognostic biomarker(s) provides for an accurate assessment of the prognosis of the prostate cancer in a mammal. Non-limiting examples of experiments can include generation of a knockout or a knock-in animal, and study of the prognosis of the knockout or knock-in animal. In some aspects, the additional experiments can include following a group of patients having prostate cancer over time and assessing the level of the biomarker in the subject over time. In some aspects, the additional experiments can include determining the level of the biomarker in an animal model of prostate cancer over time. Other experiments to validate whether the one or more identified candidate prognostic biomarker(s) will be apparent to those skilled in the field. In some aspects, the methods can further include performing an experiment to validate whether the one or more identified candidate biomarker(s) provides for an accurate assessment of the efficacy of a treatment of prostate cancer in an animal. Non-limiting examples of experiments can include administering a treatment of prostate cancer to an animal model of the prostate cancer and assessing the levels of the identified candidate biomarker in the animal model over time and assessing progression of the disease in the animal model over time. Other experiments to validate whether the one or more identified candidate biomarker(s) provides for an accurate assessment of the efficacy of a treatment of the prostate cancer in an animal will be apparent to those skilled in the field.

j. Biomarkers of Prostate Carcinoma

In some aspects, the biomarkers for prostate cancer identified herein that can be used for diagnostic, prognostic and therapeutic purposes include but are not limited to two or more of, three or more of, four or more of, five or more of, six or more of S100B, TGM4, SMOC1, LTF, ANPEP, SELE, PSCA, MT1G, ACTG2, WFDC2, SOCS3, PCP4, SYNM, GADD45B, TNC, GAS1, CNN1, ZNF185, DES, HSPB6, MSMB, MYLK, IGHG1, FLNC, ADAMTS4, MYL9, ADAM33, KRT5, PGM5, IGHV3-35, NEFH, JPH2, KRT15, TRIM29, SRD5A2, HSPB8, C11orf96, SPEG, ALDH2, FLNA, OLFM4, IGKV1D-12, TAGLN, TPM2, CRYAB, SMTN, KCNMB1, PMP22, ACTA2, and SYNPO2, or a fragment thereof. In some aspects, the biomarkers for prostate cancer identified herein that can be used for diagnostic, prognostic and therapeutic purposes include but are not limited to two or more of, three or more of, four or more of, five or more of, six or more of ADGRF1, TMEFF2, CRISP3, OR51C1P, KCNC2, PLA2G7, SPON2, CAMK2N1, NPY, KCNN2, OR51E2, AGTR1, GRIN3A, AMACR, KHDRBS3, KCNH8, ATP8A2, TMEM26, LUZP2, HPN, PLA2G2A, COL2A1, UNC80, DNASE2B, ERG, HOXC6, SPOCK1, AKR7A3, LRRN1, GOLM1, GJB1, FGFRL1, GHR, ACSM3, MCCC2, TRGV9, ZNF385B, CYP39A1, SEC11C, H2AFJ, MYO6, GPR160, BMPR1B, MBOAT2, GALNT3, CPNE4, ACER3, PLEKHH1, C9orf152, and PAQR6, or a fragment thereof. In some aspects, the biomarkers for prostate cancer identified herein that can be used for diagnostic, prognostic and therapeutic purposes include but are not limited to two or more of, three or more of, four or more or, five or more of, six or more of NPY, TMEFF2, OR51E1, FGFR3, SPON2, KCNG3, CRISP3, OR51C1P, ARG2, DEGS1, OR51E2, TRGJP2, CPNE4, GOLM1, RDH11, ACSM1, PLEKHH1, SLC30A4, TRGC1, GDF11, KHDRBS3, SPOCK1, GDF15, FGFRL1, TRGV9, LRRN1, PLA2G7, TMSB15A, KCNN2, KLK3, MARC1, PTPRT, C5orf30, COLEC12, HPN, MYO6, ADGRF1, UBE2E3, HGD, NSD2, KIF21A, HIST1H2AC, LETM1, COL9A2, BEND4, GJB1, ARFGEF3, PRAC1, F3, and SAMD5, or a fragment thereof. In some aspects, the biomarkers for prostate cancer identified herein that can be used for diagnostic, prognostic and therapeutic purposes include but are not limited to two or more of, three or more of, four or more of, five or more of, six or more of IGHA1, JCHAIN, CD79A, IGHJ6, IGKC, SAA2, CCL19, IGLC1, SAA1, IGHG2, POU2AF1, DERL3, S100A9, IGHG1, LTB, C3, MZB1, IGKV4-1, RARRES1, LTF, IGHG3, IGHJ2, IGLV3-1, LYZ, LSP1, OLFM4, FCGBP, CFB, PDZK1IP1, KRT7, CORO1A, IL7R, PRDM1, CYBA, IGHM, LCN2, SERPINF1, HCLS1, CD53, PTPRC, PTGDS, NNMT, IGFBP4, WFDC2, CP, ITGB2, C1R, CLU, KRT15, and FHL2, or a fragment thereof.

In some aspects, biomarkers for invasive carcinoma identified herein that can be used for diagnostic, prognostic and therapeutic purposes include but are not limited to two or more of ADGRF1, TMEFF2, CRISP3, OR51C1P, KCNC2, PLA2G7 and SPON2. In some aspects, the expression patterns of five or more of ADGRF1, TMEFF2, CRISP3, OR51C1P, KCNC2, PLA2G7 and SPON2 can be used in combination when practicing methods as described herein for diagnostic, prognostic and therapeutic purposes for prostate cancer.

In some aspects, biomarkers for acinar cell carcinoma identified herein that can be used for diagnostic, prognostic and therapeutic purposes include but are not limited to two or more of NPY, TMEFF2, OR51E, FGFR3, SPON2, KCNG3, CRISP3, OR51C1P and ARG2. In some aspects, the expression patterns of five or more of NPY, TMEFF2, OR51E, FGFR3, SPON2, KCNG3, CRISP3, OR51C1P and ARG2 can be used in combination when practicing the methods as described herein for diagnostic, prognostic and therapeutic purposes for prostate cancer.

In some aspects, expression levels of the aforementioned biomarkers can be useful alone, or in conjunction with additional traditional testing for prostate cancer, for example PSA testing, DRE testing, and the like.

k. Methods of Diagnosing Prostate Carcinomas

In some aspects, the methods can include (a) identifying an expression level of one or more diagnostic biomarkers described herein, in a biological sample from a subject; and (b) correlating the identified expression level the one or more diagnostic biomarkers in the biological sample to a prostate cancer stage by comparing the identified level to a reference level of the one or more diagnostic biomarkers. In some aspects wherein the subject has been previously diagnosed with prostate cancer, the methods can further include confirming the diagnosis of prostate cancer in the subject.

In some aspects, the methods can further include updating the subject's clinical record with the diagnosis of prostate cancer. In some aspects, the methods can further include enrolling the subject in a clinical trial. In some aspects, the methods can further include informing the subject's family of the diagnosis. In some aspects, the methods can further include assessing or referring the subject for enrollment in a supportive care plan or care facility. In some aspects, the methods can further include monitoring the subject more frequently.

l. Methods of Identifying Increased Likelihood of Developing Prostate Carcinomas

In some aspects wherein the subject has been identified as having increased likelihood of developing prostate cancer, the methods can further comprise monitoring the identified subject for the development of symptoms of prostate cancer. In some aspects, the methods can further include recording in the identified subject's clinical record that the subject has an increased likelihood of developing prostate cancer. In some aspects, the methods can further include notifying the subject's family that the subject has an increased likelihood or susceptibility of developing prostate cancer. In some aspects, the subject can be tested for the presence of genetic mutations known to be associated with risk for prostate cancer. In some aspects, the subject can be advised to avoid behavioral risk factors for prostate cancer. In some aspects, the methods can further include enrolling the subject in a clinical trial (e.g., for the early treatment and/or prevention of prostate cancer).

In some aspects, the methods can further include performing one or more tests to further determine the subject's risk of developing prostate cancer. Non-limiting examples of more tests to further determine the subject's risk of developing prostate cancer include taking a family history (e.g., where a family history of prostate cancer is indicative of an increased risk of developing prostate cancer), detecting a genetic mutation associated with prostate cancer, measuring PSA levels, and performing a DRE.

In some aspects, the methods can further include administering a treatment of prostate cancer to the subject, such as one of the treatments described herein.

m. Methods of Monitoring the Progression of Prostate Cancer

In some aspects, the methods described herein can be used to monitor progression of prostate cancer in a subject over time. Accordingly, in some aspects, the methods can include (a) determining a first level of one or more diagnostic biomarkers described herein in a first biological sample obtained from a subject at a first time point; (b) determining a second level of the one or more diagnostic biomarkers described herein in a second biological sample obtained from the subject at a second time point; (c) identifying: (i) a subject having an increased second level of the one or more diagnostic biomarkers described herein as compared to the first level of the one or more diagnostic biomarkers described herein, as having progressing prostate cancer, or (ii) a subject having about the same or a decreased second level of the one or more diagnostic biomarkers described herein as compared to the first level of the one or more diagnostic biomarkers described herein, as having static or regressing prostate cancer. In some aspects, the methods can include identifying a subject having an increased second level the one or more diagnostic biomarkers described herein as compared to the first level of the one or more diagnostic biomarkers described herein, as having progressing prostate cancer. In some aspects, the methods can include identifying a subject having about the same or a decreased second level of the one or more diagnostic biomarkers described herein as compared to the first level of the one or more diagnostic biomarkers described herein, as having static or regressing prostate cancer.

In some aspects, when the methods include identifying a subject as having progressing prostate cancer, the methods can further include administering a treatment for prostate cancer to the subject or increasing the dose of a previously administered treatment for prostate cancer to the subject. In some aspects, the methods can further include selecting a treatment for prostate cancer for the subject. In some aspects, the methods can further include administering a treatment of prostate cancer to the subject. In some aspects, a treatment for prostate cancer can be a treatment that reduces the rate of progression of the prostate cancer.

In some aspects, the methods can further include updating the subject's clinical record that the subject has progressing prostate cancer. In some aspects, the methods can further include enrolling the subject in a clinical trial. In some aspects, the methods can further include informing the subject's family of the progression of the disease. In some aspects, the methods can further include assessing or referring the subject for enrollment in a supportive care plan or care facility. In some aspects, the methods can further include monitoring the subject more frequently.

In some aspects, when the methods include identifying a subject as having static or regressing prostate cancer, the methods can include recording in the subject's clinical record that the subject has static or regressing prostate cancer. In some aspects, the methods can further include the methods can further include maintaining the dose or lowering the dose of a treatment for prostate cancer to be administered to the subject or ceasing administration of a treatment for prostate cancer to the subject. In some aspects, the methods can further include assessing or referring the subject to be discharged from a care facility.

n. Methods of Determining the Efficacy of a Treatment for Prostate Cancer in a Subject

In some aspects, the methods described in this section can be used to determine the efficacy of treatment of a treatment for prostate cancer in a subject. Accordingly, in some aspects, the method can include (a) determining a first expression level of one or more diagnostic biomarkers described herein in a first biological sample obtained from a subject at a first time point; (b) determining a second expression level of the one or more diagnostic biomarkers described herein in a second biological sample obtained from the subject at a second time point, wherein the subject is administered one or more doses of a therapeutic treatment between the first and second time points; (c) identifying: (i) the therapeutic treatment as being effective in a subject having about the same or a decreased second expression level of the one or more diagnostic biomarkers described herein, as compared to the first level of the one or more diagnostic biomarkers described herein, or (ii) the therapeutic treatment as not being effective in a subject having an increased second expression level of the one or more diagnostic biomarkers described herein, as compared to the first expression level of the one or more diagnostic biomarkers described herein. In some aspects, the method can include (a) determining a first expression level of one or more diagnostic biomarkers described herein in a first biological sample obtained from a subject at a first time point; (b) determining a second expression level of the one or more diagnostic biomarkers described herein in a second biological sample obtained from the subject at a second time point, wherein the subject is administered one or more doses of a therapeutic treatment between the first and second time points; (c) identifying: (i) the therapeutic treatment as being effective in a subject having an increased second expression level of the one or more diagnostic biomarkers described herein, as compared to the first expression level of the one or more diagnostic biomarkers described herein, or (ii) the therapeutic treatment as not being effective in a subject having about the same or a decreased second expression level of the one or more diagnostic biomarkers described herein, as compared to the first expression level of the one or more diagnostic biomarkers described herein.

In some aspects, the methods include identifying the therapeutic treatment as being effective in the subject. In some aspects, the methods can further include selecting additional doses of the therapeutic treatment for the subject. In some aspects, the methods can further include administering additional doses of the therapeutic treatment to the subject. In some aspects, the methods can further include recording in the subject's clinical record that the therapeutic treatment is effective in the subject.

In some aspects, the methods include identifying the therapeutic treatment as not being effective in the subject. In some aspects, the methods can further include selecting a different therapeutic treatment for the subject. In some aspects, the methods can further include administering a different therapeutic treatment to the subject. In some aspects, the methods can further include increasing the dose of the therapeutic treatment to be administered to the subject. In some aspects, the methods can include administering one or more additional doses of the therapeutic treatment to the subject in combination with an additional therapeutic treatment. In some aspects, the methods can further include ceasing administration of the therapeutic treatment to the subject. In some aspects, the methods can further include recording in the subject's clinical record that the therapeutic treatment is not effective in the subject. In some aspects, the methods can further include referring the patient for enrollment in a clinical trial of a different therapeutic agent.

o. Identifying a Candidate Drug Target for Prostate Cancer

In some aspects, provided herein are methods for identifying a candidate drug target for treatment of prostate cancer. A candidate drug target generally refers to a biomarker that has been identified as dysregulated, e.g., upregulated, downregulated, or otherwise having a modulated expression level, in a prostate tissue sample. Treatment of a drug or therapeutic molecule restores the levels (e.g., abundance) of the dysregulated biomarker. The methods can include (a) determining level(s) of one or more biomarker(s) in a location in a sample comprising prostate tissue obtained from an animal having a prostate cancer, (b) identifying: (i) one or more biomarker(s) showing dysregulated level(s) in the location in the sample as compared to reference level(s), and/or (ii) one or more biomarker(s) showing decreased level(s) in the location in the sample as compared to reference level(s), as candidate drug target(s) for treatment of the prostate cancer. In some aspects, a reference level of the one or more biomarker(s) is a level of the one or more biomarker(s) in a corresponding location in a sample comprising prostate tissue obtained from a control animal.

In some aspects, the methods can include identifying one or more biomarker(s) showing dysregulated level(s) in the location in the prostate tissue sample as compared to reference level(s) as candidate drug target(s) for treatment of the prostate cancer. In some aspects, the methods further include testing the ability of an inhibitor of the expression and/or activity of the one or more identified candidate drug target(s) to treat the prostate cancer in an animal (e.g., using a clinical trial, enzymatic assays, assessment of cell signaling activity, in vitro assays, ex vivo assays, or an animal model of the prostate cancer (e.g., any of the exemplary animal models of prostate cancer known in the art).

In some aspects, the methods can include identifying one or more biomarker(s) showing decreased level(s) in the location in the sample as compared to reference level(s) of the one or more biomarker(s) as candidate drug target(s) for treatment of the prostate cancer. In some aspects, the method can further include testing the ability of an agent that increases the expression and/or activity of the one or more identified candidate drug target(s) to treat the prostate cancer in an animal (e.g., using a clinical trial, enzymatic assays, assessment of cell signaling activity, in vitro assays, ex vivo assays, or an animal model of the prostate cancer).

In some aspects, the methods can further include additional studies to further validate a candidate drug target. Non-limiting examples of additional studies can include generation of a knockout or a knock-in animal, administration of an agent that activates and/or inhibits one or more biomarkers disclosed herein.

Some aspects of these methods can further include screening for a molecule that inhibits the expression and/or at least one activity of a candidate drug target (for a candidate drug target that has a level that is elevated at a location in the prostate as compared to a reference level). Some aspects of these methods can further include screening for a molecule that increases the expression and/or at least one activity of a candidate drug target (for a candidate drug target that has a level that is decreased at a location in the prostate as compared to a reference level).

Other studies to further validate a candidate drug target will be apparent to those skilled in the field.

p. Methods of Detecting Biomarker(s) in a Location in a Sample

In some aspects, methods described herein can be used to determine a level and/or at least one activity of one or more biomarkers in a location in a sample (e.g., a prostate tissue sample, or a cell culture sample). In some aspects, determining a level and/or an activity of one or more biomarkers can include any of the workflows described herein.

In some aspects, the methods can include contacting the sample with a binding agent that specifically binds to a biomarker (e.g., gDNA, mRNA, a protein, or a byproduct, degradation product, or fragment, or precursor thereof), wherein the binding agent further comprises an oligonucleotide having a sequence; and sequencing all or a portion of the sequence of the oligonucleotide or a complement thereof, from a probe specifically bound to the biomarker in the location of the sample, to determine the level of the biomarker in the location in the sample.

In some aspects, the methods can include delivering a plurality of probes to a sample (e.g., a tissue sample, for instance, affixed to a support), wherein a probe of the plurality of probes includes a protein that specifically binds to a biomarker (e.g., a protein, or a byproduct, degradation product, or fragment, or precursor thereof) in the tissue sample, wherein the protein is conjugated to an oligonucleotide having a sequence, and separating the probe specifically bound to the biomarker at the location of the tissue sample from the plurality of probes not specifically bound to the biomarker at the location of the tissue sample; and sequencing all or a portion of the sequence of the oligonucleotide or a complement thereof, from the specifically bound probe, and using the determined sequence to associate presence or abundance of the biomarker with the location of the tissue sample.

In some aspects, the methods can include delivering a plurality of probes to a sample (e.g., a tissue sample, for instance, affixed to a support), wherein a probe of the plurality of probes includes a first oligonucleotide that specifically binds to a biomarker (e.g., gDNA, mRNA, or a byproduct, degradation product, or fragment, or precursor thereof) in the tissue sample, wherein the first oligonucleotide is conjugated to a second oligonucleotide having a sequence, and separating the probe specifically bound to the biomarker at the location of the tissue sample from the plurality of probes not specifically bound to the biomarker at the location of the tissue sample; and sequencing all or a portion of the sequence of the second oligonucleotide or a complement thereof, from the specifically bound probe, and using the determined sequence to associate presence or abundance of the biomarker with the location of the tissue sample.

In some aspects, the methods can include delivering a plurality of probes to a tissue sample, wherein at least one probe of the plurality of probes comprises a protein that specifically binds to a biomarker (e.g., a protein, or a byproduct, degradation product, precursor, or fragment of any thereof) in the tissue sample, wherein the protein is conjugated to an oligonucleotide having a sequence, and wherein (i) each of the at least one probe comprises a protein that specifically binds a different biomarker of the tissue sample, and (ii) the protein of each of the at least one probe is conjugated to a different oligonucleotide having a sequence; imaging the tissue sample to identify a location of interest of the tissue sample; and sequencing all or a portion of the sequence(s) of the oligonucleotide(s) or a complement thereof, from the at least one probe specifically bound to the biomarker in the location of interest of the tissue sample, and using the determined sequence(s) to associate presence or abundance of the biomarker with the location of interest of the tissue sample.

In some aspects, the methods can include delivering a plurality of probes to a tissue sample, wherein at least one probe of the plurality of probes comprises a first oligonucleotide that specifically binds a biomarker (e.g., gDNA, mRNA, or a byproduct, degradation product, or fragment, or precursor thereof) in the tissue sample, wherein the first oligonucleotide is conjugated to a second oligonucleotide having a sequence, and wherein (i) each of the at least one probe comprises a first oligonucleotide that specifically binds a different biomarker of the tissue sample, and (ii) the first oligonucleotide of each of the at least one probe is conjugated to a different second oligonucleotide having a sequence; imaging the tissue sample to identify a location of interest of the tissue sample; and sequencing all or a portion of the sequence(s) of the second oligonucleotide(s) or a complement thereof, from the at least one probe specifically bound to the biomarker in the location of interest of the tissue sample, and using the determined sequence(s) to associate presence or abundance of the biomarker with the location of interest of the tissue sample.

q. Kits

In some aspects, also provided herein are kits that include one or more reagents to detect a level of one or more of any of the biomarkers and/or candidate biomarkers described herein.

In some aspects, reagents can include one or more antibodies (and/or antigen-binding antibody fragments), labeled hybridization probes, and primers. For example, in some aspects, an antibody (and/or antigen-binding antibody fragment) can be used for visualizing one or more features of a tissue sample (e.g., by using immunofluorescence or immunohistochemistry). In some aspects, an antibody (and/or antigen-binding antibody fragment) can be an analyte binding moiety, for example, as part of an analyte capture agent.

In some aspects, labeled hybridization probes can be used for in situ sequencing of one or more biomarkers and/or candidate biomarkers. In some aspects, primers can be used for amplification (e.g., clonal amplification) of a captured oligonucleotide analyte.

In some aspects, a kit can further include instructions for performing any of the methods or steps provided herein. In some aspects, a kit can include a substrate with one or more capture probes comprising a spatial barcode and a capture domain that binds to a biological analyte from a tissue sample, and reagents to detect a biological analyte, wherein the biological analyte is any of the biomarkers of this disclosure. In some aspects, the kit further includes but is not limited to one or more antibodies (and/or antigen-binding antibody fragments), labeled hybridization probes, primers, or any combination thereof for visualizing one or more features of a tissue sample.

r. Detection of Basal, Luminal, and Immune Cells/Determining Luminal or Immune Cell Infiltration

In some aspects, methods described herein further comprise detection of various different cell types in a sample from a biological subject, e.g., detection of luminal cells in a prostate tissue sample. In some aspects, one or more luminal cells can be identified in a biological sample, e.g., prostate tissue sample, by identifying the presence of one or more analytes associated with a luminal cell. In some aspects, the analyte associated with the luminal cell is CD24 or a fragment thereof, KRT8 or a fragment thereof, and KRT18 or a fragment thereof, or combinations thereof. In some aspects, one or more basal cells can be identified in a biological sample, e.g., prostate tissue sample, by identifying the presence of one or more analytes associated with a basal cell. In some aspects, the analyte associated with the basal cell is TP63 or a fragment thereof, KRT5 or a fragment thereof, KRT14 or a fragment thereof, or combinations thereof.

In some aspects, one or more immune cells can be identified in a biological sample, e.g., prostate tissue sample, by identifying the presence of one or more analytes associated with an immune cell. In some aspects, the analyte associated with the immune cell is CD3D or a fragment thereof, CD3E or a fragment thereof, CD4 or a fragment thereof, CD8A or a fragment thereof, CD247 or a fragment thereof. In some aspects, the immune cell is a B cell, a T cell, an NK cell, a monocyte, a macrophage, a neutrophil, a granulocyte, an innate lymphoid cell, or a dendritic cell. In some aspects, the one or more immune cells is: a CD3⁺ and CD4⁺T cell; a CD3⁺ and CD8⁺ T cell; a regulatory T cell comprising one or more of: CD4, Foxp3, IL17RB, CTLA4, FANK1, HAVCR1, CD25, GITR, LAG-3, and CD127; a TH1 cell comprising one or more of: CD4, CD3D, S100A4, IL7R, and IFNG; a TH2 cell comprising one or more of: CD4, IL7R, ICOS, CTLA4, TNFRSF4, and TNFRS18; a TH17 cell comprising one or more of: CD4, CD3D, IL17A, GZMA, and S100A4; a cytotoxic T cell comprising one or more of: CD8, CD3D, S100A4, IFNG, GZMB, GZMA, and IL2RB; a plasma B cell comprising: one or more JCHAIN, IGLC1, IGHA1, IGHG1, and IGKC; a monocyte comprising CD14⁺ CD16⁻; a monocyte comprising CD14⁻ CD16⁺; a natural killer cell comprising NKG7 and NCAM1; or combinations thereof.

In some aspects, the immune cell is a B cell or a plasma B cell. In some aspects, the immune cell is a tumor infiltrating B cell (TIB). In some aspects, the TIB is a plasma cell comprising one or more of: MZB1, IGLL5, IGHA1, IGHG1, JCHAIN, IGKC, IGHA2, IGLC2, IGLV3-1, and IGLV2-14; an Ig⁺ B cells comprising one or more of: IGHV3-74, SOCS3, JCHAIN, and SPARC; an activated B cell comprising: CD79B, HMGB2, HMGB1, HMGN1, and RGS13; a B cell comprising one or more of: MEF2B, RGS13, and MS4A1; a B cell comprising CD79A and CD79B, or combinations thereof.

In some aspects, the presence, location, and/or amount of a given cell type, e.g., luminal cell, of a biological sample can be determined. In some aspects, a method of determining luminal cell presence in a location of a biological sample comprises: (a) generating a dataset from the biological sample, wherein the dataset comprises one or more of: (i) analyte data for a plurality of analytes captured from a plurality of spatial locations in the biological sample; (ii) image data comprising images of the plurality of spatial locations of the biological sample; and; and (iii) registration data linking the analyte data to the image data; (b) using the dataset to identify the luminal cells in the biological sample. In some aspects, a method of determining immune cell presence in a location of a biological sample comprises: (a) generating a dataset from the biological sample, wherein the dataset comprises one or more of: (i) analyte data for a plurality of analytes captured from a plurality of spatial locations in the biological sample; (ii) image data comprising images of the plurality of spatial locations of the biological sample; and; and (iii) registration data linking the analyte data to the image data; (b) using the dataset to identify the immune cells in the biological sample.

s. Blocking Probes

In some aspects, an analyte capture sequence of a capture agent barcode domain is blocked prior to adding the analyte capture agent to a biological sample. In some aspects, an analyte capture sequence of a capture agent barcode domain is blocked prior to adding the analyte capture agent to a capture probe array. In some aspects, blocking probes are added to blocking buffer or other solutions applied in an IHC and/or IF protocol. In some aspects, a blocking probe is used to block or modify the free 3′ end of the capture agent barcode domain. In some aspects, a blocking probe is used to block or modify the free 3′ end of the analyte capture sequence of the capture agent barcode domain. In some aspects, a blocking probe can be hybridized to the analyte capture sequence of a capture agent barcode domain to mask the free 3′ end of the capture agent barcode domain. In some aspects, a blocking probe can be a hairpin probe or partially double stranded probe. In some aspects, the free 3′ end of the analyte capture sequence of the capture agent barcode domain can be blocked by chemical modification, e.g., addition of an azidomethyl group as a chemically reversible capping moiety such that the capture probes do not include a free 3′ end. Blocking or modifying the capture agent barcode domains, particularly at the free 3′ end of the capture agent barcode domain, prior to contacting the analyte capture agents with the substrate, prevents binding of the analyte capture sequence to capture probe capture domain (e.g., prevents the binding of an analyte capture sequence poly(A) tail to a poly(T) capture domain).

In some aspects, a blocking probe is used to block or modify the free 3′ end of a capture probe. In some aspects, a blocking probe is used to block or modify the free 3′ end of a capture probe capture domain. In some aspects, the analyte capture sequence is blocked prior to adding the analyte capture agent to a capture probe array. In some aspects, blocking probes are added to blocking buffer or other solutions applied in an IHC and/or IF protocol. In some aspects, a blocking probe can be hybridized to the capture domain to mask the free 3′ end of the capture domain. In some aspects, a blocking probe can be a hairpin probe or partially double stranded probe. In some aspects, the free 3′ end of the capture domain can be blocked by chemical modification, e.g., addition of an azidomethyl group as a chemically reversible capping moiety such that the capture probes do not include a free 3′ end. Blocking or modifying the capture domains, particularly at the free 3′ end of the capture domain, prior to contacting the analyte capture agents with the capture probe array, prevents binding of the analyte capture sequence to capture probe capture domain (e.g., prevents the binding of an analyte capture sequence poly(A) tail to a poly(T) capture domain).

In some aspects, the blocking probes can be reversibly removed. For example, blocking probes can be applied to block the free 3′ end of either or both the capture agent barcode domain and/or the capture probes. Blocking interaction between the analyte capture agent and the capture probe array can reduce non-specific background staining in IHC and/or IF applications. After the analyte binding agents are bound to the target analyte, the blocking probes can be removed from the 3′ end of the capture agent barcode domain and/or the capture probe, and the analyte-bound analyte binding agents can migrate to and become bound by the capture probe array. In some aspects, the removal includes denaturing the blocking probe from the analyte binding moiety barcode and/or capture probe. In some aspects, the removal includes removing a chemically reversible capping moiety. In some aspects, the removal includes digesting the blocking probe with an RNAse (e.g., RNAse H).

In some aspects, the blocking probes are oligo (dT) blocking probes. In some aspects, the oligo (dT) blocking probes can have a length of 15-30 nucleotides. In some aspects, the oligo (dT) blocking probes can have a length of 10-50 nucleotides, e.g., 10-50, 10-45, 10-40, 10-35, 10-30, 10-25, 10-20, 10-15, 15-50, 15-45, 15-40, 15-35, 15-30, 15-25, 15-20, 20-50, 20-45, 20-40, 20-35, 20-30, 20-25, 25-50, 25-45, 25-40, 25-35, 25-30, 30-50, 30-45, 30-40, 30-35, 35-50, 35-45, 35-40, 40-50, 40-45, or 45-50 nucleotides. In some aspects, the analyte capture agents can be blocked at different temperatures (e.g., 4° C. and 37° C.). In some aspects, the analyte capture agents can be blocked from binding to the capture probes more effectively at lower temperatures when using shorter blocking probes.

t. Spatially-Tagged Capture Agents

In some aspects, a spatially-tagged analyte capture agent interacts with an analyte (e.g., an analyte in a sample) and with a capture probe to identify the spatial location of the analyte. In some aspects, a spatially-tagged analyte capture agent can be an analyte capture agent with an extended capture agent barcode domain that includes a sequence complementary to a spatial barcode of a capture probe. In some aspects, an analyte capture agent is introduced to an analyte and a capture probe at the same time. In some aspects, an analyte capture agent is introduced to an analyte and a capture probe at different times. In some aspects, the spatially-tagged analyte capture agent is denatured from the capture probe before the biological sample is introduced. In some aspects, the spatially-tagged analyte capture agent binds to a biological analyte within a biological sample before the spatially-tagged analyte capture agent is denatured from the capture probe. In some aspects, the capture probe is cleaved from the substrate while attached to the spatially-tagged analyte capture agent. In some aspects, once the capture domain of the capture probe is bound to the analyte binding moiety barcode, the analyte capture sequence is extended towards the 3′ tail to include a sequence that is complementary to the sequence of the capture probe spatial barcode (e.g., producing a spatially-tagged analyte capture agent).

For example, an analyte capture agent can be introduced to a biological sample, wherein the analyte binding moiety binds to a target analyte, and then the biological sample can be treated to release the analyte-bound analyte capture agent from the sample. The analyte-bound analyte capture agent can then migrate and bind to a capture probe capture domain, and the analyte-bound capture agent barcode domain can be extended to generate a spatial barcode complement at the end of the capture agent barcode domain. The analyte-bound spatially-tagged analyte capture agent can be denatured from the capture probe, and analyzed using methods described herein.

In another example, an analyte capture agent can be hybridized to a capture probe capture domain on a capture probe array, wherein the capture agent barcode domain is extended to include a sequence complementary to the spatial barcode of the capture probe. A biological sample can be contacted with the analyte capture agent modified capture probe array. Analytes from the biological sample can be released from the sample, migrated to the analyte capture agent modified capture probe array, and captured by an analyte binding moiety. The capture agent barcode domain of the analyte-bound analyte capture agents can be denatured from the capture probe, and the biological sample can be dissociated and spatially processed according to methods described herein.

In some aspects, a spatially-tagged analyte capture agent can attach to a surface of a cell through a combination of lipophilic and covalent attachment. For example, a spatially-tagged analyte capture agent can include an oligonucleotide attached to a lipid to target the oligonucleotide to a cell membrane, and an amine group that can be covalently linked to a cell surface protein(s) via any number of chemistries described herein. In these aspects, the lipid can increase the surface concentration of the oligonucleotide and can promote the covalent reaction.

u. Slides, Biological Samples, and Analytes

In some aspects, methods described herein comprise determining the expression level of one or more analytes, e.g., one or more analytes indicative of prostate cancer, e.g., one or more analytes indicative of luminal cells, in a biological sample using a substrate (e.g., a first substrate) that includes a plurality of capture probes, where a capture probe of the plurality of capture probes include a capture domain but no spatial barcode. In some aspects, the capture probe is affixed to the substrate at a 5′ end. In some aspects, the plurality of capture probes are uniformly distributed on a surface of the substrate. In some aspects, the plurality of capture probes are located on a surface of the substrate but are not distributed on the substrate according to a pattern. In some aspects, the substrate (e.g., a second substrate) includes a plurality of capture probes, where a capture probe of the plurality of capture probes includes a capture domain and a spatial barcode.

In some aspects, the capture domain includes a sequence that is at least partially complementary to the analyte or the analyte derived molecule. In some aspects, the capture domain of the capture probe includes a poly(T) sequence. In some aspects, the capture domain includes a functional domain. In some aspects, the functional domain includes a primer sequence. In some aspects, the capture probe includes a cleavage domain. In some aspects, the cleavage domain includes a cleavable linker from the group consisting of a photocleavable linker, a UV-cleavable linker, an enzyme-cleavable linker, or a pH-sensitive cleavable linker.

In some aspects, the biological sample includes a FFPE sample. In some aspects, the biological sample includes a tissue section. In some aspects, the biological sample includes a fresh frozen sample. In some aspects, the biological sample includes live cells.

As described herein, the methods provided can be applied to analyte or analyte derived molecules including, without limitation, a second strand cDNA molecule (“second strand”). In some aspects, the analyte or analyte derived molecules include RNA and/or DNA. In some aspects, the analyte is a protein.

In some aspects, methods described herein comprise determining the abundance and/or spatial location of analyte associated with a luminal or immune infiltrating cell. Non-limiting examples of analyte associated with an immune infiltrating cell include: BLK, CD19, FCRL2, MS4A1, KIAA0125, TNFRSF17, TCL1A, SPIB, PNOC, PTRPC, PRF1, GZMA, GZMB, NKG7, GZMH, KLRK1, KLRB1, KLRD1, CTSW, GNLY, CCL13, CD209, HSD11B1, LAG3, CD244, EOMES, PTGER4, CD68, CD84, CD163, MS4A4A, TPSB2, TPSAB1, CPA3, MS4A2, HDC, FPR1, SIGLECS, CSF3R, FCAR, FCGR3B, CEACAM3, S100A12, KIR2DL3, KIR3DL1, KIR3DL2, IL21R, XCL1, XCL2, NCR1, CD6, CD3D, CD3E, SH2D1A, TRAT1, CD3G, TBX21, FOXP3, CD8A, CD8B, CD79A, CD79B, CD4, IGHA1, IGHG2, JCHAIN, IGKC, CD27, CD38, CD16, IL17RB, FANK1, CTLA4, MSR1, MRC1, NKG7, FCN1, and TIGIT/LAG3. In some aspects, the methods of determining cell infiltration in the biological sample includes identifying abundance and/or spatial location of an analyte associated with an infiltrating cell in a biological sample and further includes determining the abundance and/or spatial location of a housekeeping analyte. Non-limiting examples of housekeeping analytes that can be used in the methods described herein are as described in Eisenberg et al., Trends in Genetics, 29(10): 569-574 (2013) and Waxman et al., BMC Genomics, 8:243 (2007), the entire contents of each are incorporated herein by reference. In some aspects, a housekeeping analyte can include, without limitations, glyceraldehyde-3-phosphate dehydrogenase (GAPDH), TATA-binding protein (TBP), and ribosomal proteins (RP). In some aspects, the method includes identifying the ratio of one or more analyte associated with an immune infiltrating cell to a housekeeping analyte in the biological sample (e.g., in one or more cancerous regions).

In some aspects, the infiltrating cell is a tumor infiltrating lymphocyte (TIL), for example a T cell, and/or a B cell (TIB) (e.g., any of the exemplary B cells described herein, including plasma cells). Non-limiting examples of TILs are as described in Guo et al., (J. Oncol., doi: 10.1155/2019/2592419 (2019), the entire contents of which are incorporated herein by reference. In some aspects, the TIL is selected from: (i) a CD3⁺ and CD4⁺T cell; (ii) a CD3⁺ and CD8⁺ T cell; (iii) a regulatory T cell comprising one or more of: CD4, Foxp3, IL17RB, CTLA4, FANK1, HAVCR1, CD25, CTLA-4, GITR, LAG-3, and CD127; (iv) a TH1 cell comprising one or more of: CD4, CD3D, S100A4, IL7R, and IFNG; (v) a TH2 cell comprising one or more of: CD4, IL7R, ICOS, CTLA4, TNFRSF4, and TNFRS18; (vi) a TH17 cell comprising one or more of: CD4, CD3D, IL17A, GZMA, and S100A4; and (vii) a cytotoxic T cell comprising one or more of: CD8, CD3D, S100A4, IFNG, GZMB, GZMA, and IL2RB.

In some aspects, cell is a tumor infiltrating B cell. In some aspects, the tumor infiltrating B cell (TIB) is selected from: (i) a plasma cell comprising one or more of: MZB1, IGLL5, IGHA1, IGHG1, JCHAIN, IGKC, IGHA2, IGLC2, IGLV3-1, and IGLV2-14; (ii) an Ig⁺ B cells comprising one or more of: IGHV3-74, SOCS3, JCHAIN, and SPARC; (iii) an activated B cell comprising: CD79B, HMGB2, HMGB1, HMGN1, and RGS13; and (iv) a B cells comprising one or more of: MEF2B, RGS13, and MS4A1.

Furthermore, in some aspects the methods described herein comprise identifying abundance and/or spatial location of an infiltrating immune cell, where an infiltrating immune cell includes, without limitation, adaptive immune cells (e.g., a T cell or a B cell) and innate immune cells (e.g., Natural Killer (NK) cells, macrophages (e.g., tumor-associated macrophages (TAMs)), monocytes and dendritic cells (DCs). Non-limiting examples of infiltrating cells are as described in Zhang et al. (Cellul. Mol. Immuno., 17: 808-821 (2020)), which is herein incorporated by reference in its entirety.

In some aspects, the immune infiltrating cell is an NK cell. NK cells are innate lymphoid cells that play a role in host immune response against tumor growth. NK cells can include the attributes as described in Melaiu et al., Front. Immunol., 10:1-18 (2020) and Zhang et al., Front. Immunol. 11: 1242 (2020), the entire contents of each are incorporated herein by reference. Presence of tumor-infiltrating NK cells has been linked with a good prognosis in multiple human solid tumors. In some aspects, the NK cell is associated with an NKG7 analyte or an NCAM1 analyte, or a combination thereof.

In some aspects, the infiltrating immune cells include, but are not limited to, naïve B cells, memory B cells, plasma cells (e.g., a marker for a plasma cells includes, without limitation, CD79A, CD79B, CD38, CD27, MZB1, IGHA1, IGHG1, JCHAIN, and IGKC) CD8 T cells, CD4 naïve T cells, CD4 memory-resting T cells, CD4 memory-activated T cells, follicular helper T cells, regulatory T cells (Tregs) (e.g., a marker for a Treg includes, without limitation, FOXP3, IL17RB, CTLA4, FANK1, and CD4), gamma-delta T cells, resting NK cells, activated NK cells, monocytes, M0 macrophages, M1 macrophages, M2 macrophages, tissue associated macrophages (TAMs) (e.g., a marker for TAM includes, without limitation, CD163, MSR1, and MRC1), resting dendritic cells, activated dendritic cells, resting mast cells, activated mast cells, eosinophils, neutrophils and any combinations thereof. In some aspects, monocyte markers can include, without limitation, CD14, CD16, and FCN1 or any combination thereof. In some aspects, a T cell marker includes, without limitation, CD3D, CD3E, and CD4 or any combination thereof. In some aspects, individual T cell markers include, without limitation, CD4, CD8, TIGIT, and LAG3. In some aspects, a B cell marker includes, without limitation, CD19, CD79A, and CD79B or any combination thereof. In some aspects, a cancer marker can include, without limitation, BRCA1 and BRCA2 or any combination thereof

In some aspects, the method also includes identifying the ratio of one or more TILs to one or more TIBs in the biological sample. One skilled in the art would appreciate the ratio to cover the inverse ratio of TIB to TIL. The ratio of TILs to TIBs can include a ratio for a region of interest within the biological sample. In some cases, the region of interest can encompass the biological sample. One or more ratios of TILs to TIBs can be calculated for a biological sample. For example, each of two or more regions of interest each include a ratio of TILs to TIBs. In some aspects, the ratio of TILs to TIBs can linked to a prognostic outcome.

In some aspects, the method also includes identifying the ratio of one or more tumor infiltrating T cells to one or more TIBs in the biological sample. One skilled in the art would appreciate the ratio to cover the inverse ratio of TIB to tumor infiltrating T cells. The ratio of tumor infiltrating T cells to TIBs can include a ratio for a region of interest within the biological sample. In some cases, the region of interest can encompass the biological sample. One or more ratios of tumor infiltrating T cells to TIBs can be calculated for a biological sample. For example, each of two or more regions of interest each include a ratio of tumor infiltrating T cells to TIBs. In some aspects, the ratio of tumor infiltrating T cells to TIBs can be linked to a prognostic outcome.

In some aspects, the method for determining luminal or immune cell infiltration includes the identifying abundance and/or spatial location of an analyte associated with a cancerous region. Non-limiting examples of analytes associated with a cancerous region include: SCGB2A1, MKI67, BRCA1, BRCA2, PIKCD, CALML6, MYC, TP53, PALB2, RAD51, and MSH2. Additional non-limiting examples of analytes associated with a cancerous region include SCGB2A1, MKI67, BRCA1, BRCA2, PIK3CD, and CALML6. Other non-limiting examples of such analytes are described in www.cancer.gov/about-cancer/diagnosis-staging/diagnosis/tumor-markers-list, which is hereby incorporated by reference in its entirety. In some aspects, the analyte associated with the cancerous region is selected from the group consisting of an analyte from the AKT pathway, an analyte from the JAK-STAT pathway, and an analyte from the Notch pathway.

In some aspects, the method further includes contacting the biological sample with one or more stains. In some aspects, the one or more stains comprise a histology stain (e.g., any of the histology stains described herein or known in the art). In some aspects, the one or more stains comprises hematoxylin and eosin. In some aspects, the one or more stains comprise one or more optical labels (e.g., any of the optical labels described herein). In some aspects, the one or more optical labels are selected from the group consisting of: fluorescent, radioactive, chemiluminescent, calorimetric, or colorimetric labels.

In some aspects, the method further includes identifying one or more cancerous regions in the biological sample using the one or more stains of the biological sample. In some aspects, the method further comprises determining a prognosis of the cancer in a subject based on the abundance and/or location of the TIL in the biological sample. In some aspects, the method further includes scoring or determining the severity of the cancer in the subject based on the abundance and/or location of the TIL in the biological sample.

v. Therapeutic Methods

In some aspects, the methods can further include selecting a treatment for the subject. In some aspects, the methods can further include administering a treatment of prostate cancer to the subject. In some aspects, a treatment of prostate cancer can be a treatment that reduces the rate of progression of prostate cancer.

In some aspects, the treatment comprises surgery. In some aspects, the surgery comprises radical (open) prostatectomy, pelvic lymphadenectomy, robotic prostatectomy, or laparoscopic prostatectomy. In some aspects, the surgery comprises bilateral orchiectomy. In some aspects, the surgery comprises transurethral resection of the prostate (TURP).

In some aspects, the treatment comprises radiation therapy. In some aspects, the radiation therapy is external-beam radiation therapy, brachytherapy, intensity-modulated radiation therapy (IMRT), or proton therapy, or a combination of any one or more of the foregoing. In some aspects, the treatment comprises cryosurgery. In some aspects, the treatment comprises high-intensity focused ultrasound (HIFU).

In some aspects, the treatment comprises hormonal therapy. In some aspects, the treatment comprises administering one or more luteinizing hormone-releasing hormone (LHRH) antagonists, such as, for example, degarelix or relugolix. In some aspects, the hormonal therapy comprises administering one or more androgen receptor (AR) inhibitors. In some aspects, the AR inhibitor is apalutamide, darolutamide, enzalutamide, bicalutamide, flutamide, or nilutamide. In some aspects, the hormonal therapy comprises administering one or more androgen synthesis inhibitors. In some aspects, the androgen synthesis inhibitor is abiraterone acetate, ketoconazole, abiraterone acetate plus prednisone, ketoconazole plus prednisone, or combinations thereof. In some aspects, the hormonal therapy comprises one or more androgen receptor inhibitors in combination with bilateral orchiectomy or one or more LHRH agonists.

In some aspects, the treatment comprises a targeted therapy, such as, for example, olaparib or rucaparib. In some aspects, the treatment comprises chemotherapy, such as, for example, docetaxel, cabazitaxel, mitoxantrone, or estramustine. In some aspects, the treatment comprises immunotherapy, such as, for example, sipuleucel-T. In some aspects, the treatment comprises radiation therapy by infusion, such as, for example, infusion of radium-223.

In some aspects, the treatment comprises a bone-modifying compound. In some aspects, the bone-modifying compound is denosumab, zoledronic acid, alendronate, risedronate, ibandronate, or pamidronate.

In some aspects, the treatment administered is based on the stage of prostate cancer identified or the likelihood of prostate cancer developing in a subject. For instance, in some aspects, the prostate cancer is stage I, and the treatment is surgery, radiation therapy, cryosurgery, high-intensity focused ultrasound (HIFU), hormonal therapy, targeted therapies, chemotherapy, immunotherapy, radiation therapy by infusion, or bone-modifying compounds, or a combination of one or more of the foregoing. In some aspects, the prostate cancer is stage I, and the treatment is brachytherapy, radical prostatectomy, radiation therapy, one or more androgen receptor inhibitors, docetaxel, pelvic lymphadenectomy, or combinations of one or more of the foregoing. In some aspects, the prostate cancer is stage IIA, IIB, or IIC, and the treatment is surgery, radiation therapy, cryosurgery, high-intensity focused ultrasound (HIFU), hormonal therapy, targeted therapies, chemotherapy, immunotherapy, radiation therapy by infusion, or bone-modifying compounds, or a combination of one or more of the foregoing. In some aspects, the prostate cancer is stage IIA, IIB, or IIC, and the treatment is one or more androgen receptor inhibitors, radical prostatectomy, docetaxel, pelvic lymphadenectomy, or combinations of one or more of the foregoing. In some aspects, the prostate cancer is stage IIIA, IIIB, or IIIC, and the treatment is surgery, radiation therapy, cryosurgery, high-intensity focused ultrasound (HIFU), hormonal therapy, targeted therapies, chemotherapy, immunotherapy, radiation therapy by infusion, or bone-modifying compounds, or a combination of one or more of the foregoing. In some aspects, the prostate cancer is stage IIIA, IIIB, or IIIC, and the treatment is one or more androgen receptor inhibitors, apalutamide, darolutamide, enzalutamide, or a combination of one or more of the foregoing. In some aspects, the prostate cancer is stage IVA or IVB and the treatment is surgery, radiation therapy, cryosurgery, high-intensity focused ultrasound (HIFU), hormonal therapy, targeted therapies, chemotherapy, immunotherapy, radiation therapy by infusion, or bone-modifying compounds, or a combination of one or more of the foregoing. In some aspects, the prostate cancer is stage IVA or IVB and the treatment is one or more androgen receptor inhibitors, abiraterone, docetaxel, enzalutamide, apalutamide, radiation therapy, one or more bone modifying compounds, cabazitaxel, radiation therapy by infusion of radium-223, or a combination of one or more of the foregoing. In some aspects, the prostate cancer is metastatic castration-sensitive prostate cancer, and the treatment is docetaxel plus hormonal therapy, abiraterone with prednisone or prednisolone plus hormonal therapy, apalutamide plus hormonal therapy, or enzalutamide plus hormonal therapy. In some aspects, the prostate cancer is non-metastatic castration-resistant prostate cancer and the treatment is surgery and/or hormonal therapy. In some aspects, the prostate cancer is metastatic castration-resistant prostate cancer, and the treatment is AR inhibitors, targeted therapies, chemotherapy, immunotherapy, radiation therapy, or bone-modifying compounds.

In some aspects, the treatment comprises one or more compounds that are administered in an amount of from 0.1 mg/kg body weight to 1000 mg/kg body weight. In some aspects, the one or more compounds is administered in an amount of 0.1 mg/kg body weight or less, 0.25 mg/kg body weight or less, 0.50 mg/kg body weight or less, 0.75 mg/kg body weight or less, 1 mg/kg body weight or less, 2 mg/kg body weight or less, 3 mg/kg body weight or less, 4 mg/kg body weight or less, 5 mg/kg body weight or less, 6 mg/kg body weight or less, 7 mg/kg body weight or less, 8 mg/kg body weight or less, 9 mg/kg body weight or less, 10 mg/kg body weight or less, 15 mg/kg body weight or less, 20 mg/kg body weight or less, 25 mg/kg body weight or less, 30 mg/kg body weight or less, 35 mg/kg body weight or less, 40 mg/kg body weight or less, 45 mg/kg body weight or less, 50 mg/kg body weight or less, 60 mg/kg body weight or less, 70 mg/kg body weight or less, 80 mg/kg body weight or less, 90 mg/kg body weight or less, 100 mg/kg body weight or less, or 200 mg/kg body weight or less. In some aspects, the one or more compounds is administered in an amount of from about 0.1 mg/kg body weight to about 100 mg/kg body weight, about 0.5 mg/kg body weight to about 50 mg/kg body weight, about 1.0 mg/kg body weight to about 40 mg/kg body weight, about 1.0 to about 30.0 mg/kg body weight, about 1.0 mg/kg body weight to about 25 mg/kg body weight, about 1.0 mg/kg body weight to about 20 mg/kg body weight, about 1.0 mg/kg body weight to about 15 mg/kg body weight, about 2.0 to about 30 mg/kg body weight, about 2.0 to about 25 mg/kg body weight, about 2.0 to about 20 mg/kg body weight, about 2.0 to about 15 mg/kg body weight, about 3.0 to about 30 mg/kg body weight, about 3.0 to about 25 mg/kg body weight, about 3.0 to about 20 mg/kg body weight, about 3.0 to about 15 mg/kg body weight, about 4.0 to about 30 mg/kg body weight, about 4.0 to about 25 mg/kg body weight, about 4.0 to about 20 mg/kg body weight, about 4.0 to about 15 mg/kg body weight, about 5.0 to about 30 mg/kg body weight, about 5.0 to about 25 mg/kg body weight, about 5.0 to about 20 mg/kg body weight, or about 5.0 to about 15 mg/kg body weight.

In some aspects, radiation therapy is administered locally to a tumor lesion to enhance the local immunogenicity of a subject's tumor (adjuvinating radiation) and/or to kill tumor cells (ablative radiation). In some aspects, radiation therapy is administered systemically to a subject. In some aspects, the radiation therapy is tomotherapy, stereotactic radiation, intensity-modulated radiation therapy (IMRT), hypofractionated radiotherapy, hypoxia-guided radiotherapy, and/or proton therapy. In some aspects, radiation is followed by administration of a second therapy (e.g., chemotherapy, immunotherapy). In some aspects, radiation is provided concurrently with administration of a second therapy (e.g., chemotherapy, immunotherapy).

In some aspects, any of the above therapeutic agents are provided before, substantially contemporaneous with, or after other modes of treatment, for example, surgery, chemotherapy, radiation therapy, or the administration of a biologic, such as another therapeutic antibody. In some aspects, the cancer has recurred or progressed following a therapy selected from surgery, chemotherapy, and radiation therapy, or a combination thereof.

In some aspects, the treatment comprises one or more antibodies. In some aspects, the treatment comprises one or more chimeric antigen receptors. In some aspects, the treatment comprises AVASTIN® (bevacizumab) either alone or in combination with one or more additional treatments. In some aspects, for treatment of cancer, as discussed herein, the antibodies are administered in conjunction with one or more additional anti-cancer agents, such as the chemotherapeutic agent, growth inhibitory agent, anti-angiogenesis agent and/or anti-neoplastic composition.

In some aspects, the methods can further include updating the subject's clinical record with the diagnosis of prostate cancer. In some aspects, the methods can further include enrolling the subject in a clinical trial. In some aspects, the methods can further include informing the subject's family of the diagnosis. In some aspects, the methods can further include assessing or referring the subject for enrollment in a supportive care plan or care facility. In some aspects, the methods can further include monitoring the subject more frequently.

In some aspects, the methods can further comprise monitoring the identified subject for the development of symptoms of prostate cancer. In some aspects, the methods can further include recording in the identified subject's clinical record that the subject has an increased likelihood of developing prostate cancer. In some aspects, the methods can further include notifying the subject's family that the subject has an increased likelihood or susceptibility of developing prostate cancer.

In some aspects, the methods can further include administering to the subject a treatment for decreasing the rate of progression or decreasing the likelihood of developing prostate cancer. In some aspects, a treatment of cancer can include surgery, radiation therapy, chemotherapy, surgery, radiation therapy, chemotherapy, targeted drug therapy, and tumor treating fields (TTF) therapy. In some aspects, the subject can be tested for the presence of genetic mutations known to be associated with risk for prostate cancer.

In some aspects, the methods can further include performing one or more tests to further determine the subject's risk of developing prostate cancer. Non-limiting examples of more tests to further determine the subject's risk of developing cancer include detecting a genetic mutation associated with prostate cancer, PSA test, DRE, prostate biopsy with pathologist annotation, and determining the levels of other biomarkers indicative of an increased risk of developing prostate cancer.

In some aspects, the methods can further include updating the subject's clinical record to indicate an increased risk of developing prostate cancer. In some aspects, the methods can further include enrolling the subject in a clinical trial (e.g., for the early treatment and/or prevention of prostate cancer). In some aspects, the methods can further include informing the subject's family of the subject's likelihood of developing prostate cancer. In some aspects, the methods can further include monitoring the subject more frequently.

w. Prophylactic Methods

In some aspects, the methods can further include selecting a prophylactic for preventing and/or reducing the incidence of prostate cancer in a subject having an increased likelihood of developing prostate cancer.

In some aspects, the prophylactic comprises surgery. In some aspects, the surgery comprises radical (open) prostatectomy, pelvic lymphadenectomy, robotic prostatectomy, or laparoscopic prostatectomy. In some aspects, the surgery comprises bilateral orchiectomy. In some aspects, the surgery comprises transurethral resection of the prostate (TURP).

In some aspects, the prophylactic comprises radiation therapy. In some aspects, the radiation therapy is external-beam radiation therapy, brachytherapy, intensity-modulated radiation therapy (IMRT), or proton therapy, or a combination of any one or more of the foregoing. In some aspects, the treatment comprises cryosurgery. In some aspects, the treatment comprises high-intensity focused ultrasound (HIFU).

In some aspects, the prophylactic comprises hormonal therapy. In some aspects, the prophylactic comprises administering one or more luteinizing hormone-releasing hormone (LHRH) antagonists, such as, for example, degarelix or relugolix. In some aspects, the hormonal therapy comprises administering one or more androgen receptor (AR) inhibitors. In some aspects, the AR inhibitor is apalutamide, darolutamide, enzalutamide, bicalutamide, flutamide, or nilutamide. In some aspects, the hormonal therapy comprises administering one or more androgen synthesis inhibitors. In some aspects, the androgen synthesis inhibitor is abiraterone acetate, ketoconazole, abiraterone acetate plus prednisone, ketoconazole plus prednisone, or combinations thereof. In some aspects, the hormonal therapy comprises one or more androgen receptor inhibitors in combination with bilateral orchiectomy or one or more LHRH agonists.

In some aspects, the prophylactic comprises a targeted therapy, such as, for example, olaparib or rucaparib. In some aspects, the prophylactic comprises chemotherapy, such as, for example, docetaxel, cabazitaxel, mitoxantrone, or estramustine. In some aspects, the prophylactic comprises immunotherapy, such as, for example, sipuleucel-T. In some aspects, the prophylactic comprises radiation therapy by infusion, such as, for example, infusion of radium-223.

In some aspects, the prophylactic comprises a bone-modifying compound. In some aspects, the bone-modifying compound is denosumab, zoledronic acid, alendronate, risedronate, ibandronate, or pamidronate.

In some aspects, the prophylactic comprises one or more antibodies. In some aspects, the prophylactic comprises one or more chimeric antigen receptors. In some aspects, for prophylaxis of cancer, as discussed herein, the antibodies are administered in conjunction with one or more additional anti-cancer agents, such as the chemotherapeutic agent, growth inhibitory agent, anti-angiogenesis agent and/or anti-neoplastic composition.

x. Preparation of Biological Samples

A variety of steps can be performed to prepare a biological sample, such as a prostate tissue sample, for analysis. Except where indicated otherwise, the preparative steps described below can generally be combined in any manner to appropriately prepare a particular sample for analysis.

i. Tissue Sectioning

A biological sample can be harvested from a subject (e.g., via surgical biopsy, whole subject sectioning), grown in vitro on a growth substrate or culture dish as a population of cells, or prepared as a tissue slice or tissue section. Grown samples may be sufficiently thin for analysis without further processing steps. Alternatively, grown samples, and samples obtained via biopsy or sectioning, can be prepared as thin tissue sections using a mechanical cutting apparatus such as a vibrating blade microtome. As another alternative, in some aspects, a thin tissue section can be prepared by applying a touch imprint of a biological sample to a suitable substrate material.

The thickness of the tissue section can be a fraction of (e.g., less than 0.9, 0.8, 0.7, 0.6, 0.5, 0.4, 0.3, 0.2, or 0.1) the maximum cross-sectional dimension of a cell. However, tissue sections having a thickness that is larger than the maximum cross-section cell dimension can also be used. For example, cryostat sections can be used, which can be, e.g., 5-20 micrometers thick.

More generally, the thickness of a tissue section typically depends on the method used to prepare the section and the physical characteristics of the tissue, and therefore sections having a wide variety of different thicknesses can be prepared and used. For example, the thickness of the tissue section can be at least 0.1, 0.2, 0.3, 0.4, 0.5, 0.7, 1.0, 1.5, 2, 3, 4, 5, 6, 7, 8, 9, 10, 12, 13, 14, 15, 20, 30, 40, or 50 micrometers. Thicker sections can also be used if desired or convenient, e.g., at least 70, 80, 90, or 100 micrometers or more. Typically, the thickness of a tissue section is between 1-100 micrometers, 1-50 micrometers, 1-30 micrometers, 1-25 micrometers, 1-20 micrometers, 1-15 micrometers, 1-10 micrometers, 2-8 micrometers, 3-7 micrometers, or 4-6 micrometers, but as mentioned above, sections with thicknesses larger or smaller than these ranges can also be analyzed.

Multiple sections can also be obtained from a single biological sample. For example, multiple tissue sections can be obtained from a surgical biopsy sample by performing serial sectioning of the biopsy sample using a sectioning blade. Spatial information among the serial sections can be preserved in this manner, and the sections can be analyzed successively to obtain three-dimensional information about the biological sample.

ii. Freezing

In some aspects, the biological sample (e.g., a tissue section as described above) can be prepared by deep freezing at a temperature suitable to maintain or preserve the integrity (e.g., the physical characteristics) of the tissue structure. Such a temperature can be, e.g., less than −20° C., or less than −25° C., −30° C., −40° C., −50° C., −60° C., −70° C., 80° C. -90° C., −100° C., −110° C., −120° C., −130° C., −140° C., −150° C., −160° C., −170° C., −180° C., −190° C., or −200° C. The frozen tissue sample can be sectioned, e.g., thinly sliced, onto a substrate surface using any number of suitable methods. For example, a tissue sample can be prepared using a chilled microtome (e.g., a cryostat) set at a temperature suitable to maintain both the structural integrity of the tissue sample and the chemical properties of the nucleic acids in the sample. Such a temperature can be, e.g., less than −15° C., less than −20° C., or less than −25° C. A sample can be snap frozen in isopentane and liquid nitrogen. Frozen samples can be stored in a sealed container prior to embedding.

iii. Formalin Fixation and Paraffin Embedding

In some aspects, the biological sample can be prepared using formalin-fixation and paraffin-embedding (FFPE), which are established methods in the field. In some aspects, cell suspensions and other non-tissue samples can be prepared using formalin-fixation and paraffin-embedding. Following fixation of the sample and embedding in a paraffin or resin block, the sample can be sectioned as described above. Prior to analysis, the paraffin-embedding material can be removed from the tissue section (e.g., deparaffinization) by incubating the tissue section in an appropriate solvent (e.g., xylene) followed by a rinse (e.g., 99.5% ethanol for 2 minutes, 96% ethanol for 2 minutes, and 70% ethanol for 2 minutes).

iv. Fixation

As an alternative to formalin fixation described above, a biological sample can be fixed in any of a variety of other fixatives to preserve the biological structure of the sample prior to analysis. For example, a sample can be fixed via immersion in ethanol, methanol, acetone, formaldehyde (e.g., 2% formaldehyde), paraformaldehyde-Triton, glutaraldehyde, or combinations thereof.

In some aspects, acetone fixation is used with fresh frozen samples, which can include, but are not limited to, prostate tissue samples. In some aspects, a compatible fixation method is chosen and/or optimized based on a desired workflow. For example, formaldehyde fixation may be chosen as compatible for workflows using IHC/IF protocols for protein visualization. As another example, methanol fixation may be chosen for workflows emphasizing RNA/DNA library quality. Acetone fixation may be chosen in some applications to permeabilize the tissue. When acetone fixation is performed, pre-permeabilization steps (described below) may not be performed. Alternatively, acetone fixation can be performed in conjunction with permeabilization steps. As another example, a fresh frozen tissue sample may be fixed with formaldehyde (e.g., 2% formaldehyde) in PBS. In some embodiments, a fresh frozen tissue sample fixed with formaldehyde may undergo a decrosslinking step. In some embodiments, a fresh frozen tissue sample fixed with formaldehyde may subsequently be washed with a wash buffer. Non-limiting examples of wash buffers include TE buffer of pH 9, and SSC buffer.

v. Embedding

As an alternative to paraffin embedding described above, a biological sample can be embedded in any of a variety of other embedding materials to provide a substrate to the sample prior to sectioning and other handling steps. In general, the embedding material is removed prior to analysis of tissue sections obtained from the sample. Suitable embedding materials include, but are not limited to, waxes, resins (e.g., methacrylate resins), epoxies, and agar.

vi. Staining

To facilitate visualization, biological samples can be stained using a wide variety of stains and staining techniques. In some aspects, a sample can be stained using any number of biological stains, including but not limited to, acridine orange, Bismarck brown, carmine, coomassie blue, cresyl violet, DAPI, eosin, ethidium bromide, acid fuchsine, hematoxylin, Hoechst stains, iodine, methyl green, methylene blue, neutral red, Nile blue, Nile red, osmium tetroxide, propidium iodide, rhodamine, or safranin.

The sample can be stained using known staining techniques, including Can-Grunwald, Giemsa, hematoxylin and eosin (H&E), Jenner's, Leishman, Masson's trichrome, Papanicolaou, Romanowsky, silver, Sudan, Wright's, and/or Periodic Acid Schiff (PAS) staining techniques. PAS staining is typically performed after formalin or acetone fixation.

In some aspects, the biological sample can be stained using a detectable label (e.g., radioisotopes, fluorophores, chemiluminescent compounds, bioluminescent compounds, and dyes) as described elsewhere herein. In some aspects, a biological sample is stained using only one type of stain or one technique. In some aspects, staining includes biological staining techniques such as H&E staining. In some aspects, staining includes identifying analytes using fluorescently-conjugated antibodies. In some aspects, a biological sample is stained using two or more different types of stains, or two or more different staining techniques. For example, a biological sample can be prepared by staining and imaging using one technique (e.g., H&E staining and brightfield imaging), followed by staining and imaging using another technique (e.g., IHC/IF staining and fluorescence microscopy) for the same biological sample.

In some aspects, biological samples can be destained. Methods of destaining or discoloring a biological sample are known in the art, and generally depend on the nature of the stain(s) applied to the sample. For example, H&E staining can be destained by washing the sample in HCl, or any other low pH acid (e.g., selenic acid, sulfuric acid, hydroiodic acid, benzoic acid, carbonic acid, malic acid, phosphoric acid, oxalic acid, succinic acid, salicylic acid, tartaric acid, sulfurous acid, trichloroacetic acid, hydrobromic acid, hydrochloric acid, nitric acid, orthophosphoric acid, arsenic acid, selenous acid, chromic acid, citric acid, hydrofluoric acid, nitrous acid, isocyanic acid, formic acid, hydrogen selenide, molybdic acid, lactic acid, acetic acid, carbonic acid, hydrogen sulfide, or combinations thereof). In some aspects, destaining can include 1, 2, 3, 4, 5, or more washes in a low pH acid (e.g., HCl). In some aspects, destaining can include adding HCl to a downstream solution (e.g., permeabilization solution). In some aspects, destaining can include dissolving an enzyme used in the disclosed methods (e.g., pepsin) in a low pH acid (e.g., HCl) solution. In some aspects, after destaining hematoxylin with a low pH acid, other reagents can be added to the destaining solution to raise the pH for use in other applications. For example, SDS can be added to a low pH acid destaining solution in order to raise the pH as compared to the low pH acid destaining solution alone. As another example, in some aspects, one or more immunofluorescence stains are applied to the sample via antibody coupling. Such stains can be removed using techniques such as cleavage of disulfide linkages via treatment with a reducing agent and detergent washing, chaotropic salt treatment, treatment with antigen retrieval solution, and treatment with an acidic glycine buffer. Methods for multiplexed staining and destaining are described, for example, in Bolognesi et al., J. Histochem. Cytochem. 2017; 65(8): 431-444, Lin et al., Nat Commun. 2015; 6:8390, Pirici et al., J. Histochem. Cytochem. 2009; 57:567-75, and Glass et al., J. Histochem. Cytochem. 2009; 57:899-905, the entire contents of each of which are incorporated herein by reference.

vii. Hydrogel Embedding

In some aspects, hydrogel formation occurs within a biological sample. In some aspects, a biological sample (e.g., tissue section) is embedded in a hydrogel. In some aspects, hydrogel subunits are infused into the biological sample, and polymerization of the hydrogel is initiated by an external or internal stimulus. A “hydrogel” as described herein can include a cross-linked 3D network of hydrophilic polymer chains. A “hydrogel subunit” can be a hydrophilic monomer, a molecular precursor, or a polymer that can be polymerized (e.g., cross-linked) to form a three-dimensional (3D) hydrogel network.

A hydrogel can swell in the presence of water. In some aspects, a hydrogel comprises a natural material. In some aspects, a hydrogel includes a synthetic material. In some aspects, a hydrogel includes a hybrid material, e.g., the hydrogel material comprises elements of both synthetic and natural polymers. Any of the materials used in hydrogels or hydrogels comprising a polypeptide-based material described herein can be used. Embedding the sample in this manner typically involves contacting the biological sample with a hydrogel such that the biological sample becomes surrounded by the hydrogel. For example, the sample can be embedded by contacting the sample with a suitable polymer material, and activating the polymer material to form a hydrogel. In some aspects, the hydrogel is formed such that the hydrogel is internalized within the biological sample.

In some aspects, the biological sample is immobilized in the hydrogel via cross-linking of the polymer material that forms the hydrogel. Cross-linking can be performed chemically and/or photochemically, or alternatively by any other hydrogel-formation method known in the art. For example, the biological sample can be immobilized in the hydrogel by polyacrylamide crosslinking. Further, analytes of a biological sample can be immobilized in a hydrogel by crosslinking (e.g., polyacrylamide crosslinking).

The composition and application of the hydrogel to a biological sample typically depends on the nature and preparation of the biological sample (e.g., sectioned, non-sectioned, fresh-frozen tissue, type of fixation). A hydrogel can be any appropriate hydrogel where upon formation of the hydrogel on the biological sample the biological sample becomes anchored to or embedded in the hydrogel. Non-limiting examples of hydrogels are described herein or are known in the art. As one example, where the biological sample is a tissue section, the hydrogel can include a monomer solution and an ammonium persulfate (APS) initiator/tetramethylethylenediamine (TEMED) accelerator solution. As another example, where the biological sample consists of cells (e.g., cultured cells or cells disassociated from a tissue sample), the cells can be incubated with the monomer solution and APS/TEMED solutions. For cells, hydrogel are formed in compartments, including but not limited to devices used to culture, maintain, or transport the cells. For example, hydrogels can be formed with monomer solution plus APS/TEMED added to the compartment to a depth ranging from about 0.1 μm to about 5 mm.

In some aspects, a hydrogel includes a linker that allows anchoring of the biological sample to the hydrogel. In some aspects, a hydrogel includes linkers that allow anchoring of biological analytes to the hydrogel. In such cases, the linker can be added to the hydrogel before, contemporaneously with, or after hydrogel formation. Non-limiting examples of linkers that anchor nucleic acids to the hydrogel can include 6-((Acryloyl)amino) hexanoic acid (Acryloyl-X SE) (available from ThermoFisher, Waltham, Mass.), Label-IT Amine (available from MirusBio, Madison, Wis.) and Label X (Chen et al., Nat. Methods 13:679-684, (2016)).

In some aspects, functionalization chemistry can be used. In some aspects, functionalization chemistry includes hydrogel-tissue chemistry (HTC). Any hydrogel-tissue backbone (e.g., synthetic or native) suitable for HTC can be used for anchoring biological macromolecules and modulating functionalization. Non-limiting examples of methods using HTC backbone variants include CLARITY, PACT, ExM, SWITCH and ePACT. In some aspects, hydrogel formation within a biological sample is permanent. For example, biological macromolecules can permanently adhere to the hydrogel allowing multiple rounds of interrogation. In some aspects, hydrogel formation within a biological sample is reversible.

In some aspects, additional reagents are added to the hydrogel subunits before, contemporaneously with, and/or after polymerization. For example, additional reagents can include but are not limited to oligonucleotides (e.g., capture probes), endonucleases to fragment DNA, fragmentation buffer for DNA, DNA polymerase enzymes, dNTPs used to amplify the nucleic acid and to attach the barcode to the amplified fragments. Other enzymes can be used, including without limitation, RNA polymerase, transposase, ligase, proteinase K, and DNAse. Additional reagents can also include reverse transcriptase enzymes, including enzymes with terminal transferase activity, primers, and switch oligonucleotides. In some aspects, optical labels are added to the hydrogel subunits before, contemporaneously with, and/or after polymerization.

In some aspects, HTC reagents are added to the hydrogel before, contemporaneously with, and/or after polymerization. In some aspects, a cell tagging agent is added to the hydrogel before, contemporaneously with, and/or after polymerization. In some aspects, a cell-penetrating agent is added to the hydrogel before, contemporaneously with, and/or after polymerization.

In some aspects, a biological sample is embedded in a hydrogel to facilitate sample transfer to another location (e.g., to an array). For example, archived biological samples (e.g., FFPE tissue sections) can be transferred from storage to a spatial array to perform spatial analysis. In some aspects, a biological sample on a substrate can be covered with any of the prepolymer solutions described herein. In some aspects, the prepolymer solution can be polymerized such that a hydrogel is formed on top of and/or around the biological sample. Hydrogel formation can occur in a manner sufficient to anchor (e.g., embed) the biological sample to the hydrogel. After hydrogel formation, the biological sample is anchored to (e.g., embedded in) the hydrogel wherein separating the hydrogel from the substrate (e.g., glass slide) results in the biological sample separating from the substrate along with the hydrogel. The biological sample contained in the hydrogel can then be contacted with a spatial array, and spatial analysis can be performed on the biological sample.

Any variety of characteristics can determine the transfer conditions required for a given biological sample. Non-limiting examples of characteristics likely to impact transfer conditions include the sample (e.g., thickness, fixation, and cross-linking) and/or the analyte of interest (different conditions to preserve and/or transfer different analytes (e.g., DNA, RNA, and protein)).

In some aspects, the hydrogel is removed after contacting the biological sample with the spatial array. For example, methods described herein can include an event-dependent (e.g., light or chemical) depolymerizing hydrogel, wherein upon application of the event (e.g., external stimuli) the hydrogel depolymerizes. In one example, a biological sample can be anchored to a DTT-sensitive hydrogel, where addition of DTT can cause the hydrogel to depolymerize and release the anchored biological sample.

Hydrogels embedded within biological samples can be cleared using any suitable method. For example, electrophoretic tissue clearing methods can be used to remove biological macromolecules from the hydrogel-embedded sample. In some aspects, a hydrogel-embedded sample is stored in a medium before or after clearing of hydrogel (e.g., a mounting medium, methylcellulose, or other semi-solid mediums).

In some aspects, the hydrogel chemistry can be tuned to specifically bind (e.g., retain) particular species of analytes (e.g., RNA, DNA, protein, etc.). In some aspects, a hydrogel includes a linker that allows anchoring of the biological sample to the hydrogel. In some aspects, a hydrogel includes linkers that allow anchoring of biological analytes to the hydrogel. In such cases, the linker can be added to the hydrogel before, contemporaneously with, or after hydrogel formation. Non-limiting examples of linkers that anchor nucleic acids to the hydrogel can include 6-((Acryloyl)amino) hexanoic acid (Acryloyl-X SE), Label-IT Amine and Label X (Chen et al., Nat. Methods 13:679-684, (2016)). Non-limiting examples of characteristics likely to impact transfer conditions include the sample (e.g., thickness, fixation, and cross-linking) and/or the analyte of interest (different conditions to preserve and/or transfer different analytes (e.g., DNA, RNA, and protein)).

Additional methods and aspects of hydrogel embedding of biological samples are described for example in Chen et al., Science 347(6221):543-548, 2015, the entire contents of which are incorporated herein by reference.

viii. Biological Sample Transfer

In some aspects, a biological sample immobilized on a substrate (e.g., a biological sample prepared using methanol fixation or formalin-fixation and paraffin-embedding (FFPE)) is transferred to a spatial array using a hydrogel. In some aspects, a hydrogel is formed on top of a biological sample on a substrate (e.g., glass slide). For example, hydrogel formation can occur in a manner sufficient to anchor (e.g., embed) the biological sample to the hydrogel. After hydrogel formation, the biological sample is anchored to (e.g., embedded in) the hydrogel wherein separating the hydrogel from the substrate results in the biological sample separating from the substrate along with the hydrogel. The biological sample can then be contacted with a spatial array, thereby allowing spatial profiling of the biological sample. In some aspects, the hydrogel is removed after contacting the biological sample with the spatial array. For example, methods described herein can include an event-dependent (e.g., light or chemical) depolymerizing hydrogel, wherein upon application of the event (e.g., external stimuli) the hydrogel depolymerizes. In one example, a biological sample can be anchored to a DTT-sensitive hydrogel, where addition of DTT can cause the hydrogel to depolymerize and release the anchored biological sample. A hydrogel can be any appropriate hydrogel where upon formation of the hydrogel on the biological sample the biological sample becomes anchored to or embedded in the hydrogel. Non-limiting examples of hydrogels are described herein or are known in the art. In some aspects, a hydrogel includes a linker that allows anchoring of the biological sample to the hydrogel. In some aspects, a hydrogel includes linkers that allow anchoring of biological analytes to the hydrogel. In such cases, the linker can be added to the hydrogel before, contemporaneously with, or after hydrogel formation. Non-limiting examples of linkers that anchor nucleic acids to the hydrogel can include 6-((Acryloyl)amino) hexanoic acid (Acryloyl-X SE) (available from ThermoFisher, Waltham, Mass.), Label-IT Amine (available from MirusBio, Madison, Wis.) and Label X (Chen et al., Nat. Methods 13:679-684, 2016). Any variety of characteristics can determine the transfer conditions required for a given biological sample. Non-limiting examples of characteristics likely to impact transfer conditions include the sample (e.g., thickness, fixation, and cross-linking) and/or the analyte of interest (different conditions to preserve and/or transfer different analytes (e.g., DNA, RNA, and protein)). In some aspects, hydrogel formation can occur in a manner sufficient to anchor the analytes (e.g., embed) in the biological sample to the hydrogel. In some aspects, the hydrogel can be imploded (e.g., shrunk) with the anchored analytes (e.g., embedded in the hydrogel) present in the biological sample. In some aspects, the hydrogel can be expanded (e.g., isometric expansion) with the anchored analytes (e.g., embedded in the hydrogel) present in the biological sample. In some aspects, the hydrogel can be imploded (e.g., shrunk) and subsequently expanded with anchored analytes (e.g., embedded in the hydrogel) present in the biological sample.

ix. Isometric Expansion

In some aspects, a biological sample embedded in a hydrogel can be isometrically expanded. Isometric expansion methods that can be used include hydration, a preparative step in expansion microscopy, as described in Chen et al., Science 347(6221):543-548, 2015; Asano et al. Current Protocols. 2018, 80:1, doi:10.1002/cpcb.56 and Gao et al. BMC Biology. 2017, 15:50, doi:10.1186/s12915-017-0393-3, Wassie, A.T., et al, Expansion microscopy: principles and uses in biological research, Nature Methods, 16(1): 33-41 (2018), each of which is incorporated by reference in its entirety.

In general, the steps used to perform isometric expansion of the biological sample can depend on the characteristics of the sample (e.g., thickness of tissue section, fixation, cross-linking), and/or the analyte of interest (e.g., different conditions to anchor RNA, DNA, and protein to a gel).

Isometric expansion can be performed by anchoring one or more components of a biological sample to a gel, followed by gel formation, proteolysis, and swelling. Isometric expansion of the biological sample can occur prior to immobilization of the biological sample on a substrate, or after the biological sample is immobilized to a substrate. In some aspects, the isometrically expanded biological sample can be removed from the substrate prior to contacting the expanded biological sample with a spatially barcoded array (e.g., spatially barcoded capture probes on a substrate).

In some aspects, proteins in the biological sample are anchored to a swellable gel such as a polyelectrolyte gel. An antibody can be directed to the protein before, after, or in conjunction with being anchored to the swellable gel. DNA and/or RNA in a biological sample can also be anchored to the swellable gel via a suitable linker. Examples of such linkers include, but are not limited to, 6-((Acryloyl)amino) hexanoic acid (Acryloyl-X SE) (available from ThermoFisher, Waltham, Mass.), Label-IT Amine (available from MirusBio, Madison, Wis.) and Label X (described for example in Chen et al., Nat. Methods 13:679-684, 2016, the entire contents of which are incorporated herein by reference).

Isometric expansion of the sample can increase the spatial resolution of the subsequent analysis of the sample. For example, isometric expansion of the biological sample can result in increased resolution in spatial profiling (e.g., single-cell profiling). The increased resolution in spatial profiling can be determined by comparison of an isometrically expanded sample with a sample that has not been isometrically expanded.

Isometric expansion can enable three-dimensional spatial resolution of the subsequent analysis of the sample. In some aspects, isometric expansion of the biological sample can occur in the presence of spatial profiling reagents (e.g., analyte capture agents or capture probes). For example, the swellable gel can include analyte capture agents or capture probes anchored to the swellable gel via a suitable linker. In some aspects, spatial profiling reagents can be delivered to particular locations in an isometrically expanded biological sample.

In some aspects, a biological sample is isometrically expanded to a volume at least 2×, 2.1×, 2.2×, 2.3×, 2.4×, 2.5×, 2.6×, 2.7×, 2.8×, 2.9×, 3×, 3.1×, 3.2×, 3.3×, 3.4×, 3.5×, 3.6×, 3.7×, 3.8×, 3.9×, 4×, 4.1×, 4.2×, 4.3×, 4.4×, 4.5×, 4.6×, 4.7×, 4.8×, or 4.9× its non-expanded volume. In some aspects, the sample is isometrically expanded to at least 2× and less than 20× of its non-expanded volume.

In some aspects, a biological sample embedded in a hydrogel is isometrically expanded to a volume at least 2×, 2.1×, 2.2×, 2.3×, 2.4×, 2.5×, 2.6×, 2.7×, 2.8×, 2.9×, 3×, 3.1×, 3.2×, 3.3×, 3.4×, 3.5×, 3.6×, 3.7×, 3.8×, 3.9×, 4×, 4.1×, 4.2×, 4.3×, 4.4×, 4.5×, 4.6×, 4.7×, 4.8×, or 4.9× its non-expanded volume. In some aspects, the biological sample embedded in a hydrogel is isometrically expanded to at least 2× and less than 20× of its non-expanded volume.

x. Substrate Attachment

In some aspects, the biological sample can be attached to a substrate. Examples of substrates suitable for this purpose are described in detail below. Attachment of the biological sample can be irreversible or reversible, depending upon the nature of the sample and subsequent steps in the analytical method.

In certain aspects, the sample can be attached to the substrate reversibly by applying a suitable polymer coating to the substrate, and contacting the sample to the polymer coating. The sample can then be detached from the substrate using an organic solvent that at least partially dissolves the polymer coating. Hydrogels are examples of polymers that are suitable for this purpose.

More generally, in some aspects, the substrate can be coated or functionalized with one or more substances to facilitate attachment of the sample to the substrate. Suitable substances that can be used to coat or functionalize the substrate include, but are not limited to, lectins, poly-lysine, antibodies, and polysaccharides.

xi. Tissue Permeabilization

In some aspects, a biological sample can be permeabilized to facilitate transfer of analytes out of the sample, and/or to facilitate transfer of species (such as capture probes) into the sample. If a sample is not permeabilized sufficiently, the amount of analyte captured from the sample may be too low to enable adequate analysis. Conversely, if the tissue sample is too permeable, the relative spatial relationship of the analytes within the tissue sample can be lost. Hence, a balance between permeabilizing the tissue sample enough to obtain good signal intensity while still maintaining the spatial resolution of the analyte distribution in the sample is desirable.

In general, a biological sample can be permeabilized by exposing the sample to one or more permeabilizing agents. Suitable agents for this purpose include, but are not limited to, organic solvents (e.g., acetone, ethanol, and methanol), cross-linking agents (e.g., paraformaldehyde), detergents (e.g., saponin, Triton X-100™, Tween-20™, or sodium dodecyl sulfate (SDS)), and enzymes (e.g., trypsin, proteases (e.g., proteinase K). In some aspects, the detergent is an anionic detergent (e.g., SDS or N-lauroylsarcosine sodium salt solution). In some aspects, the biological sample can be permeabilized using any of the methods described herein (e.g., using any of the detergents described herein, e.g., SDS and/or N-lauroylsarcosine sodium salt solution) before or after enzymatic treatment (e.g., treatment with any of the enzymes described herein, e.g., trypin, proteases (e.g., pepsin and/or proteinase K)).

In some aspects, a biological sample can be permeabilized by exposing the sample to greater than about 1.0 w/v % (e.g., greater than about 2.0 w/v %, greater than about 3.0 w/v %, greater than about 4.0 w/v %, greater than about 5.0 w/v %, greater than about 6.0 w/v %, greater than about 7.0 w/v %, greater than about 8.0 w/v %, greater than about 9.0 w/v %, greater than about 10.0 w/v %, greater than about 11.0 w/v %, greater than about 12.0 w/v %, or greater than about 13.0 w/v %) sodium dodecyl sulfate (SDS) and/or N-lauroylsarcosine or N-lauroylsarcosine sodium salt. In some aspects, a biological sample can be permeabilized by exposing the sample (e.g., for about 5 minutes to about 1 hour, about 5 minutes to about 40 minutes, about 5 minutes to about 30 minutes, about 5 minutes to about 20 minutes, or about 5 minutes to about 10 minutes) to about 1.0 w/v % to about 14.0 w/v % (e.g., about 2.0 w/v % to about 14.0 w/v %, about 2.0 w/v % to about 12.0 w/v %, about 2.0 w/v % to about 10.0 w/v %, about 4.0 w/v % to about 14.0 w/v %, about 4.0 w/v % to about 12.0 w/v %, about 4.0 w/v % to about 10.0 w/v %, about 6.0 w/v % to about 14.0 w/v %, about 6.0 w/v % to about 12.0 w/v %, about 6.0 w/v % to about 10.0 w/v %, about 8.0 w/v % to about 14.0 w/v %, about 8.0 w/v % to about 12.0 w/v %, about 8.0 w/v % to about 10.0 w/v %, about 10.0% w/v % to about 14.0 w/v %, about 10.0 w/v % to about 12.0 w/v %, or about 12.0 w/v % to about 14.0 w/v %) SDS and/or N-lauroylsarcosine salt solution and/or proteinase K (e.g., at a temperature of about 4% to about 35° C., about 4° C. to about 25° C., about 4° C. to about 20° C., about 4° C. to about 10° C., about 10° C. to about 25° C., about 10° C. to about 20° C., about 10° C. to about 15° C., about 35° C. to about 50° C., about 35° C. to about 45° C., about 35° C. to about 40° C., about 40° C. to about 50° C., about 40° C. to about 45° C., or about 45° C. to about 50° C.).

In some aspects, the biological sample can be incubated with a permeabilizing agent to facilitate permeabilization of the sample. Additional methods for sample permeabilization are described, for example, in Jamur et al., Method Mol. Biol. 588:63-66, 2010, the entire contents of which are incorporated herein by reference.

xii. Permeabilization Reagents

In some aspects, the biological sample can be permeabilized by adding one or more permeabilization reagents to the sample. Examples of suitable permeabilization agents include, but are not limited to, bioactive reagents such as permeabilization enzymes that are used for permeabilizing different cell types.

Permeabilization reagents be added to the biological sample to facilitate permeabilization. For example, surfactant-based permeabilization solutions can be used to permeabilize sample cells. Permeabilization solutions can include ionic surfactants such as, for example, sarcosyl and sodium dodecyl sulfate (SDS). More generally, chemical permeabilization agents can include, without limitation, organic solvents, chelating agents, detergents, surfactants, and chaotropic agents.

xiii. Proteases

In some aspects, a medium, solution, or permeabilization solution may contain one or more proteases. In some aspects, a biological sample treated with a protease capable of degrading histone proteins can result in the generation of fragmented genomic DNA. The fragmented genomic DNA can be captured using the same capture domain (e.g., capture domain having a poly(T) sequence) used to capture mRNA. In some aspects, a biological sample is treated with a protease capable of degrading histone proteins and an RNA protectant prior to spatial profiling in order to facilitate the capture of both genomic DNA and mRNA.

In some aspects, a biological sample is permeabilized by exposing the sample to a protease capable of degrading histone proteins. As used herein, the term “histone protein” typically refers to a linker histone protein (e.g., H1) and/or a core histone protein (e.g., H2A, H2B, H3, and H4). In some aspects, a protease degrades linker histone proteins, core histone proteins, or linker histone proteins and core histone proteins. Any suitable protease capable of degrading histone proteins in a biological sample can be used. Non-limiting examples of proteases capable of degrading histone proteins include proteases inhibited by leupeptin and TLCK (Tosyl-L-lysyl-chloromethane hydrochloride), a protease encoded by the EUO gene from Chlamydia trachomatis serovar A, granzyme A, a serine protease (e.g., trypsin or trypsin-like protease, neutral serine protease, elastase, cathepsin G), an aspartyl protease (e.g., cathepsin D), a peptidase family Cl enzyme (e.g., cathepsin L), pepsin, proteinase K, a protease that is inhibited by the diazomethane inhibitor Z-Phe-Phe-CHN(2) or the epoxide inhibitor E-64, a lysosomal protease, or an azurophilic enzyme (e.g., cathepsin G, elastase, proteinase 3, neutral serine protease). In some aspects, a serine protease is a trypsin enzyme, trypsin-like enzyme or a functional variant or derivative thereof (e.g., P00761; COHK48; Q8IYP2; Q8BW11; Q6IE06; P35035; P00760; P06871; Q90627; P16049; P07477; P00762; P35031; P19799; P35036; Q29463; P06872; Q90628; P07478; P07146; P00763; P35032; P70059; P29786; P35037; Q90629; P35030; P08426; P35033; P35038; P12788; P29787; P35039; P35040; Q8NHM4; P35041; P35043; P35044; P54624; P04814; P35045; P32821; P54625; P35004; P35046; P32822; P35047; COHKAS; COHKA2; P54627; P35005; C0HKA6; C0HKA3; P52905; P83348; P00765; P35042; P81071; P35049; P51588; P35050; P35034; P35051; P24664; P35048; P00764; P00775; P54628; P42278; P54629; P42279; Q91041; P54630; P42280; C0HKA4) or a combination thereof. In some aspects, a trypsin enzyme is P00761, P00760, Q29463, or a combination thereof. In some aspects, a protease capable of degrading one or more histone proteins comprises an amino acid sequence with at least 80% sequence identity to P00761, P00760, or Q29463. In some aspects, a protease capable of degrading one or more histone proteins comprises an amino acid sequence with at least 85%, 90%, 91%, 92%, 93%, 94%, 95%, 96%, 97%, 98%, or 99% identity to P00761, P00760, or Q29463. A protease may be considered a functional variant if it has at least 50% e.g., at least 60%, 70%, 80%, 90%, 95%, 96%, 97%, 98%, 99%, or 100% of the activity relative to the activity of the protease in condition optimum for the enzyme. In some aspects, the enzymatic treatment with pepsin enzyme, or pepsin like enzyme, can include: P03954/PEPA1_MACFU; P28712/PEPA1_RABIT; P27677/PEPA2_MACFU; P27821/PEPA2_RABIT; P0DJD8/PEPA3_HUMAN; P27822/PEPA3_RABIT; PODJD7/PEPA4_HUMAN; P27678/PEPA4_MACFU; P28713/PEPA4_RABIT; PODJD9/PEPA5_HUMAN; Q9D106/PEPA5_MOUSE; P27823/PEPAF_RABIT; P00792/PEPA_BOVIN; Q9N2D4/PEPA_CALJA; Q9GMY6/PEPA_CANLF; P00793/PEPA_CHICK; P11489/PEPA_MACMU; P00791/PEPA_PIG; Q9GMY7/PEPA_RHIFE; Q9GMY8/PEPA_SORUN; P81497/PEPA_SUNMU; P13636/PEPA_URSTH and functional variants and derivatives thereof, or a combination thereof. In some aspects, the pepsin enzyme can include: P00791/PEPA_PIG; P00792/PEPA_BOVIN, functional variants, derivatives, or combinations thereof.

Additionally, the protease may be contained in a reaction mixture (solution), which also includes other components (e.g., buffer, salt, chelator (e.g., EDTA), and/or detergent (e.g., SDS, N-Lauroylsarcosine sodium salt solution)). The reaction mixture may be buffered, having a pH of about 6.5-8.5, e.g., about 7.0-8.0. Additionally, the reaction mixture may be used at any suitable temperature, such as about 10-50° C., e.g., about 10-44° C., 11-43° C., 12-42° C., 13-41° C., 14-40° C., 15-39° C., 16-38° C., 17-37° C., e.g., about 10° C., 12° C., 15° C., 18° C., 20° C., 22° C., 25° C., 28° C., 30° C., 33° C., 35° C. or 37° C., preferably about 35-45° C., e.g., about 37° C.

xiv. Other Reagents

In some aspects, a permeabilization solution can contain additional reagents or a biological sample may be treated with additional reagents in order to optimize biological sample permeabilization. In some aspects, an additional reagent is an RNA protectant. As used herein, the term “RNA protectant” typically refers to a reagent that protects RNA from RNA nucleases (e.g., RNases). Any appropriate RNA protectant that protects RNA from degradation can be used. A non-limiting example of a RNA protectant includes organic solvents (e.g., at least 60%, 65%, 70%, 75%, 80%, 85%, 90%, or 95% v/v organic solvent), which include, without limitation, ethanol, methanol, propan-2-ol, acetone, trichloroacetic acid, propanol, polyethylene glycol, acetic acid, or a combination thereof. In some aspects, a RNA protectant includes ethanol, methanol and/or propan-2-ol, or a combination thereof. In some aspects, a RNA protectant includes RNAlater ICE (ThermoFisher Scientific). In some aspects, the RNA protectant comprises at least about 60% ethanol. In some aspects, the RNA protectant comprises about 60-95% ethanol, about 0-35% methanol and about 0-35% propan-2-ol, wherein the total amount of organic solvent in the medium is not more than about 95%. In some aspects, the RNA protectant comprises about 60-95% ethanol, about 5-20% methanol and about 5-20% propan-2-ol, wherein the total amount of organic solvent in the medium is not more than about 95%.

In some aspects, the RNA protectant includes a salt. The salt may include ammonium sulfate, ammonium bisulfate, ammonium chloride, ammonium acetate, cesium sulfate, cadmium sulfate, cesium iron (II) sulfate, chromium (III) sulfate, cobalt (II) sulfate, copper (II) sulfate, lithium chloride, lithium acetate, lithium sulfate, magnesium sulfate, magnesium chloride, manganese sulfate, manganese chloride, potassium chloride, potassium sulfate, sodium chloride, sodium acetate, sodium sulfate, zinc chloride, zinc acetate and zinc sulfate. In some aspects, the salt is a sulfate salt, for example, ammonium sulfate, ammonium bisulfate, cesium sulfate, cadmium sulfate, cesium iron (II) sulfate, chromium (III) sulfate, cobalt (II) sulfate, copper (II) sulfate, lithium sulfate, magnesium sulfate, manganese sulfate, potassium sulfate, sodium sulfate, or zinc sulfate. In some aspects, the salt is ammonium sulfate. The salt may be present at a concentration of about 20 g/100 ml of medium or less, such as about 15g/100 ml, 10g/100 ml, 9g/100 ml, 8g/100 ml, 7g/100 ml, 6g/100 ml, 5g/100 ml or less, e.g., about 4g, 3g, 2g or lg/100ml.

Additionally, the RNA protectant may be contained in a medium that further includes a chelator (e.g., EDTA), a buffer (e.g., sodium citrate, sodium acetate, potassium citrate, or potassium acetate, preferably sodium acetate), and/or buffered to a pH between about 4-8 (e.g., about 5).

In some aspects, the biological sample is treated with one or more RNA protectants before, contemporaneously with, or after permeabilization. For example, a biological sample is treated with one or more RNA protectants prior to treatment with one or more permeabilization reagents (e.g., one or more proteases). In another example, a biological sample is treated with a solution including one or more RNA protectants and one or more permeabilization reagents (e.g., one or more proteases). In yet another example, a biological sample is treated with one or more RNA protectants after the biological sample has been treated with one or more permeabilization reagents (e.g., one or more proteases). In some aspects, a biological sample is treated with one or more RNA protectants prior to fixation.

In some aspects, identifying the location of the captured analyte in the biological sample includes a nucleic acid extension reaction. In some aspects where a capture probe captures a fragmented genomic DNA molecule, a nucleic acid extension reaction includes DNA polymerase. For example, a nucleic acid extension reaction includes using a DNA polymerase to extend the capture probe that is hybridized to the captured analyte (e.g., fragmented genomic DNA) using the captured analyte (e.g., fragmented genomic DNA) as a template. The product of the extension reaction includes a spatially-barcoded analyte (e.g., spatially-barcoded fragmented genomic DNA). The spatially-barcoded analyte (e.g., spatially-barcoded fragmented genomic DNA) can be used to identify the spatial location of the analyte in the biological sample. Any DNA polymerase that is capable of extending the capture probe using the captured analyte as a template can be used for the methods described herein. Non-limiting examples of DNA polymerases include T7 DNA polymerase; Bsu DNA polymerase; and E.coli DNA Polymerase pol I.

xv. Diffusion-Resistant Media

In some aspects, a diffusion-resistant medium, typically used to limit diffusion of analytes, can include at least one permeabilization reagent. For example, the diffusion-resistant medium (e.g., a hydrogel) can include wells (e.g., micro-, nano-, or picowells or pores) containing a permeabilization buffer or reagents. In some aspects, the diffusion-resistant medium (e.g., a hydrogel) is soaked in permeabilization buffer prior to contacting the hydrogel with a sample. In some aspects, the hydrogel or other diffusion-resistant medium can contain dried reagents or monomers to deliver permeabilization reagents when the diffusion-resistant medium is applied to a biological sample. In some aspects, the diffusion-resistant medium, (e.g., hydrogel) is covalently attached to a solid substrate (e.g., an acrylated glass slide).

In some aspects, the hydrogel can be modified to both deliver permeabilization reagents and contain capture probes. For example, a hydrogel film can be modified to include spatially-barcoded capture probes. The spatially-barcoded hydrogel film is then soaked in permeabilization buffer before contacting the spatially-barcoded hydrogel film to the sample. In another example, a hydrogel can be modified to include spatially-barcoded capture probes and designed to serve as a porous membrane (e.g., a permeable hydrogel) when exposed to permeabilization buffer or any other biological sample preparation reagent. The permeabilization reagent diffuses through the spatially-barcoded permeable hydrogel and permeabilizes the biological sample on the other side of the hydrogel. The analytes then diffuse into the spatially-barcoded hydrogel after exposure to permeabilization reagents. In such cases, the spatially-barcoded hydrogel (e.g., porous membrane) is facilitating the diffusion of the biological analytes in the biological sample into the hydrogel. In some aspects, biological analytes diffuse into the hydrogel before exposure to permeabilization reagents (e.g., when secreted analytes are present outside of the biological sample or in aspects where a biological sample is lysed or permeabilized by other means prior to addition of permeabilization reagents). In some aspects, the permeabilization reagent is flowed over the hydrogel at a variable flow rate (e.g., any flow rate that facilitates diffusion of the permeabilization reagent across the spatially-barcoded hydrogel). In some aspects, the permeabilization reagents are flowed through a microfluidic chamber or channel over the spatially-barcoded hydrogel. In some aspects, after using flow to introduce permeabilization reagents to the biological sample, biological sample preparation reagents can be flowed over the hydrogel to further facilitate diffusion of the biological analytes into the spatially-barcoded hydrogel. The spatially-barcoded hydrogel film thus delivers permeabilization reagents to a sample surface in contact with the spatially-barcoded hydrogel, enhancing analyte migration and capture. In some aspects, the spatially-barcoded hydrogel is applied to a sample and placed in a permeabilization bulk solution. In some aspects, the hydrogel film soaked in permeabilization reagents is sandwiched between a sample and a spatially-barcoded array. In some aspects, target analytes are able to diffuse through the permeabilizing reagent soaked hydrogel and hybridize or bind the capture probes on the other side of the hydrogel. In some aspects, the thickness of the hydrogel is proportional to the resolution loss. In some aspects, wells (e.g., micro-, nano-, or picowells) can contain spatially-barcoded capture probes and permeabilization reagents and/or buffer. In some aspects, spatially-barcoded capture probes and permeabilization reagents are held between spacers. In some aspects, the sample is punch, cut, or transferred into the well, wherein a target analyte diffuses through the permeabilization reagent/buffer and to the spatially-barcoded capture probes. In some aspects, resolution loss may be proportional to gap thickness (e.g., the amount of permeabilization buffer between the sample and the capture probes). In some aspects, the diffusion-resistant medium (e.g., hydrogel) is between approximately 50-500 micrometers thick including 500, 450, 400, 350, 300, 250, 200, 150, 100, or 50 micrometers thick, or any thickness within 50 and 500 micrometers.

In some aspects, a biological sample is exposed to a porous membrane (e.g., a permeable hydrogel) to aid in permeabilization and limit diffusive analyte losses, while allowing permeabilization reagents to reach a sample. Membrane chemistry and pore volume can be manipulated to minimize analyte loss. In some aspects, the porous membrane may be made of glass, silicon, paper, hydrogel, polymer monoliths, or other material. In some aspects, the material may be naturally porous. In some aspects, the material may have pores or wells etched into solid material. In some aspects, the permeabilization reagents are flowed through a microfluidic chamber or channel over the porous membrane. In some aspects, the flow controls the sample's access to the permeabilization reagents. In some aspects, the porous membrane is a permeable hydrogel. For example, a hydrogel is permeable when permeabilization reagents and/or biological sample preparation reagents can pass through the hydrogel using diffusion. Any suitable permeabilization reagents and/or biological sample preparation reagents described herein can be used under conditions sufficient to release analytes (e.g., nucleic acid, protein, metabolites, lipids, etc.) from the biological sample. In some aspects, a hydrogel is exposed to the biological sample on one side and permeabilization reagent on the other side. The permeabilization reagent diffuses through the permeable hydrogel and permeabilizes the biological sample on the other side of the hydrogel. In some aspects, permeabilization reagents are flowed over the hydrogel at a variable flow rate (e.g., any flow rate that facilitates diffusion of the permeabilization reagent across the hydrogel). In some aspects, the permeabilization reagents are flowed through a microfluidic chamber or channel over the hydrogel. Flowing permeabilization reagents across the hydrogel enables control of the concentration of reagents. In some aspects, hydrogel chemistry and pore volume can be tuned to enhance permeabilization and limit diffusive analyte losses.

In some aspects, a porous membrane is sandwiched between a spatially-barcoded array and the sample, wherein permeabilization solution is applied over the porous membrane. The permeabilization reagents diffuse through the pores of the membrane and into the biological sample. In some aspects, the biological sample can be placed on a substrate (e.g., a glass slide). Biological analytes then diffuse through the porous membrane and into to the space containing the capture probes. In some aspects, the porous membrane is modified to include capture probes. For example, the capture probes can be attached to a surface of the porous membrane using any of the methods described herein. In another example, the capture probes can be embedded in the porous membrane at any depth that allows interaction with a biological analyte. In some aspects, the porous membrane is placed onto a biological sample in a configuration that allows interaction between the capture probes on the porous membrane and the biological analytes from the biological sample. For example, the capture probes are located on the side of the porous membrane that is proximal to the biological sample. In such cases, permeabilization reagents on the other side of the porous membrane diffuse through the porous membrane into the location containing the biological sample and the capture probes in order to facilitate permeabilization of the biological sample (e.g., also facilitating capture of the biological analytes by the capture probes). In some aspects, the porous membrane is located between the sample and the capture probes. In some aspects, the permeabilization reagents are flowed through a microfluidic chamber or channel over the porous membrane.

xvii. Selective Enrichment of RNA Species

In some aspects, where RNA is the analyte, one or more RNA analyte species of interest can be selectively enriched (e.g., Adiconis, et. al., Comparative analysis of RNA sequencing methods for degraded and low-input samples, Nature, vol. 10, July 2013, 623-632, herein incorporated by reference in its entirety). For example, one or more species of RNA can be selected by addition of one or more oligonucleotides to the sample. In some aspects, the additional oligonucleotide is a sequence used for priming a reaction by a polymerase. For example, one or more primer sequences with sequence complementarity to one or more RNAs of interest can be used to amplify the one or more RNAs of interest, thereby selectively enriching these RNAs. In some aspects, an oligonucleotide with sequence complementarity to the complementary strand of captured RNA (e.g., cDNA) can bind to the cDNA. For example, biotinylated oligonucleotides with sequence complementary to one or more cDNAs of interest binds to the cDNA and can be selected using biotinylation-streptavidin affinity using any of a variety of methods known to the field (e.g., streptavidin beads).

Additionally or alternatively, one or more species of analyte (e.g., mitochondrial DNA or RNA) can be down-selected (e.g., removed) using any of a variety of methods. Methods for down-selection, such as removal of rRNA, are described in PCT/US2017/028397, hereby incorporated by reference in its entirety. In some embodiments, such down-selection of analytes that are not of interest can result in improved capture of other types of analytes that are of interest. For example, as discussed further infra, probes can be administered to a sample that selectively hybridize to ribosomal RNA (rRNA), thereby reducing the pool and concentration of rRNA in the sample.

In depletion protocols, probes can be administered to a sample that selectively hybridize to ribosomal RNA (rRNA), thereby reducing the pool and concentration of rRNA in the sample. Probes can be administered to a biological sample that selectively hybridize to mitochondria RNA (mtRNA), thereby reducing the pool and concentration of mtRNA in the sample. In some aspects, probes complementary to mitochondrial RNA can be added during cDNA synthesis, or probes complementary to both ribosomal and mitochondrial RNA can be added during cDNA synthesis. Subsequent application of capture probes to the sample can result in improved capture of other types of RNA due to a reduction in non-specific RNA (e.g., down-selected RNA) present in the sample. Additionally and alternatively, duplex-specific nuclease (DSN) treatment can remove rRNA (see, e.g., Archer, et al, Selective and flexible depletion of problematic sequences from RNA-seq libraries at the cDNA stage, BMC Genomics, 15 401, (2014), the entire contents of which are incorporated herein by reference). Furthermore, hydroxyapatite chromatography can remove abundant species (e.g., rRNA) (see, e.g., Vandernoot, V.A., cDNA normalization by hydroxyapatite chromatography to enrich transcriptome diversity in RNA-seq applications, Biotechniques, 53(6) 373-80, (2012), the entire contents of which are incorporated herein by reference).

xviii. Immunohistochemistry and Immunofluorescence

In some aspects, immunofluorescence or immunohistochemistry protocols (direct and indirect staining techniques) can be performed as a part of, or in addition to, the exemplary spatial workflows presented herein. For example, tissue sections can be fixed according to methods described herein. The biological sample can be transferred to an array (e.g., capture probe array), wherein analytes (e.g., proteins) are probed using immunofluorescence protocols. For example, the sample can be rehydrated, blocked, and permeabilized (3XSSC, 2% BSA, 0.1% Triton X, 1 U/μl RNAse inhibitor for 10 min at 4° C.) before being stained with fluorescent primary antibodies (1:100 in 3XSSC, 2% BSA, 0.1% Triton X, 1 U/μl RNAse inhibitor for 30 min at 4° C.). The biological sample can be washed, coverslipped (in glycerol+1 U/μl RNAse inhibitor), imaged (e.g., using a confocal microscope or other apparatus capable of fluorescent detection), washed, and processed according to analyte capture or spatial workflows described herein.

In some aspects, an antigen retrieval buffer can improve antibody capture in IF/IHC protocols. An exemplary protocol for antigen retrieval can be preheating the antigen retrieval buffer (e.g., to 95° C.), immersing the biological sample in the heated antigen retrieval buffer for a predetermined time, and then removing the biological sample from the antigen retrieval buffer and washing the biological sample.

In some aspects, optimizing permeabilization can be useful for identifying intracellular analytes. Permeabilization optimization can include selection of permeabilization agents, concentration of permeabilization agents, and permeabilization duration. Tissue permeabilization is discussed elsewhere herein.

In some aspects, blocking an array and/or a biological sample in preparation of labeling the biological sample decreases unspecific binding of the antibodies to the array and/or biological sample (decreases background). Some aspects provide for blocking buffers/blocking solutions that can be applied before and/or during application of the label, wherein the blocking buffer can include a blocking agent, and optionally a surfactant and/or a salt solution. In some aspects, a blocking agent can be bovine serum albumin (BSA), serum, gelatin (e.g., fish gelatin), milk (e.g., non-fat dry milk), casein, polyethylene glycol (PEG), polyvinyl alcohol (PVA), or polyvinylpyrrolidone (PVP), biotin blocking reagent, a peroxidase blocking reagent, levamisole, Carnoy's solution, glycine, lysine, sodium borohydride, pontamine sky blue, Sudan Black, trypan blue, FITC blocking agent, and/or acetic acid. The blocking buffer/blocking solution can be applied to the array and/or biological sample prior to and/or during labeling (e.g., application of fluorophore-conjugated antibodies) to the biological sample.

In some aspects, additional steps or optimizations can be included in performing IF/IHC protocols in conjunction with spatial arrays. Additional steps or optimizations can be included in performing spatially-tagged analyte capture agent workflows discussed herein.

In some aspects, provided herein are methods for spatially detecting an analyte (e.g., detecting the location of an analyte, e.g., a biological analyte) from a biological sample (e.g., an analyte present in a biological sample, such as a tissue section) that include: (a) providing a biological sample on a substrate; (b) staining the biological sample on the substrate, imaging the stained biological sample, and selecting the biological sample or subsection of the biological sample (e.g., region of interest) to subject to analysis; (c) providing an array comprising one or more pluralities of capture probes on a substrate; (d) contacting the biological sample with the array, thereby allowing a capture probe of the one or more pluralities of capture probes to capture the analyte of interest; and (e) analyzing the captured analyte, thereby spatially detecting the analyte of interest. Any variety of staining and imaging techniques as described herein or known in the art can be used in accordance with methods described herein. In some aspects, the staining includes optical labels as described herein, including, but not limited to, fluorescent, radioactive, chemiluminescent, calorimetric, or colorimetric detectable labels. In some aspects, the staining includes a fluorescent antibody directed to a target analyte (e.g., cell surface or intracellular proteins) in the biological sample. In some aspects, the staining includes an immunohistochemistry stain directed to a target analyte (e.g., cell surface or intracellular proteins) in the biological sample. In some aspects, the staining includes a chemical stain such as hematoxylin and eosin (H&E) or periodic acid-schiff (PAS). In some aspects, significant time (e.g., days, months, or years) can elapse between staining and/or imaging the biological sample and performing analysis. In some aspects, reagents for performing analysis are added to the biological sample before, contemporaneously with, or after the array is contacted to the biological sample. In some aspects, step (d) includes placing the array onto the biological sample. In some aspects, the array is a flexible array where the plurality of spatially-barcoded features (e.g., a substrate with capture probes, a bead with capture probes) are attached to a flexible substrate. In some aspects, measures are taken to slow down a reaction (e.g., cooling the temperature of the biological sample or using enzymes that preferentially perform their primary function at lower or higher temperature as compared to their optimal functional temperature) before the array is contacted with the biological sample. In some aspects, step (e) is performed without bringing the biological sample out of contact with the array. In some aspects, step (e) is performed after the biological sample is no longer in contact with the array. In some aspects, the biological sample is tagged with an analyte capture agent before, contemporaneously with, or after staining and/or imaging of the biological sample. In such cases, significant time (e.g., days, months, or years) can elapse between staining and/or imaging and performing analysis. In some aspects, the array is adapted to facilitate biological analyte migration from the stained and/or imaged biological sample onto the array (e.g., using any of the materials or methods described herein). In some aspects, a biological sample is permeabilized before being contacted with an array. In some aspects, the rate of permeabilization is slowed prior to contacting a biological sample with an array (e.g., to limit diffusion of analytes away from their original locations in the biological sample). In some aspects, modulating the rate of permeabilization (e.g., modulating the activity of a permeabilization reagent) can occur by modulating a condition that the biological sample is exposed to (e.g., modulating temperature, pH, and/or light). In some aspects, modulating the rate of permeabilization includes use of external stimuli (e.g., small molecules, enzymes, and/or activating reagents) to modulate the rate of permeabilization. For example, a permeabilization reagent can be provided to a biological sample prior to contact with an array, which permeabilization reagent is inactive until a condition (e.g., temperature, pH, and/or light) is changed or an external stimulus (e.g., a small molecule, an enzyme, and/or an activating reagent) is provided.

In some aspects, provided herein are methods for spatially detecting an analyte (e.g., detecting the location of an analyte, e.g., a biological analyte) from a biological sample (e.g., present in a biological sample such as a tissue section) that include: (a) providing a biological sample on a substrate; (b) staining the biological sample on the substrate, imaging the stained biological sample, and selecting the biological sample or subsection of the biological sample (e.g., a region of interest) to subject to spatial transcriptomic analysis; (c) providing an array comprising one or more pluralities of capture probes on a substrate; (d) contacting the biological sample with the array, thereby allowing a capture probe of the one or more pluralities of capture probes to capture the biological analyte of interest; and (e) analyzing the captured biological analyte, thereby spatially detecting the biological analyte of interest.

### EXAMPLES

### Example 1

### Gene Expression Analysis in Normal and Cancerous Prostate Tissues

In the present example, the spatial gene expression analysis of various different genes was performed in normal and cancerous prostate tissue samples. Human prostate tissue FFPE sample blocks were obtained from Indivumed GmbH tissue biobank, sectioned, and assayed for gene expression. The prostate cancer grade and stage as assessed by a pathologist using hematoxylin and eosin (H&E) staining of the samples are found in Table 1.

TABLE 1
Prostate tissue Sample Score and Grade

| Tissue Type | Gleason Score | Cancer Stage* |
|---|---|---|
| Normal | N/A | N/A |
| Adenocarcinoma - Invasive | 7 | III |
| Acinar Cell Carcinoma | 7 | IV |

*Cancer stage as assessed by a pathologist analyzing a stained prostate tissue sample.

Prostate tissue sample blocks were sectioned using a microtome into 5 μm sections and placed onto array areas on Visium FFPE Gene Expression slides per the manufacturer's instructions (Visium Gene Expression for FFPE—Tissue Preparation Guide Demonstrated Protocol G000408, 10× Genomics, Inc.). Briefly, tissue sections were deparaffinized, stained with hematoxylin and eosin (H&E), and subsequently imaged. The H&E stained tissue sections were viewed at 20× magnification for imaging, and the images obtained were sent to the AGOKO telepathology platform for manual annotation by certified pathologists.

After imaging, tissue samples were decrosslinked and target specific probes added. The target-specific probes hybridized to their respective targets and subsequently ligated together. Following probe ligation, the tissues were permeabilized and the ligated probe products were captured by capture probes affixed to the array areas of the slides following the manufacturer's protocol (Visium Spatial Gene Expression Reagents Kits User Guide CG000407, 10× Genomics, Inc.). The Visium Gene Expression for FFPE included probe pairs that targeted the whole human transcriptome. Once captured, the ligated probe products were extended to include capture probe sequences. The extended products were released from the array for sequencing library preparation and subsequent sequencing on an Illumina NovaSeq sequencer.

Sequencing data was analyzed using Space Ranger pipeline and correlated with the H&E image. Visualization of the correlation was done using the Loupe Browser. The pathologist's annotations were imported into the Loupe Browser as an additional classification category.

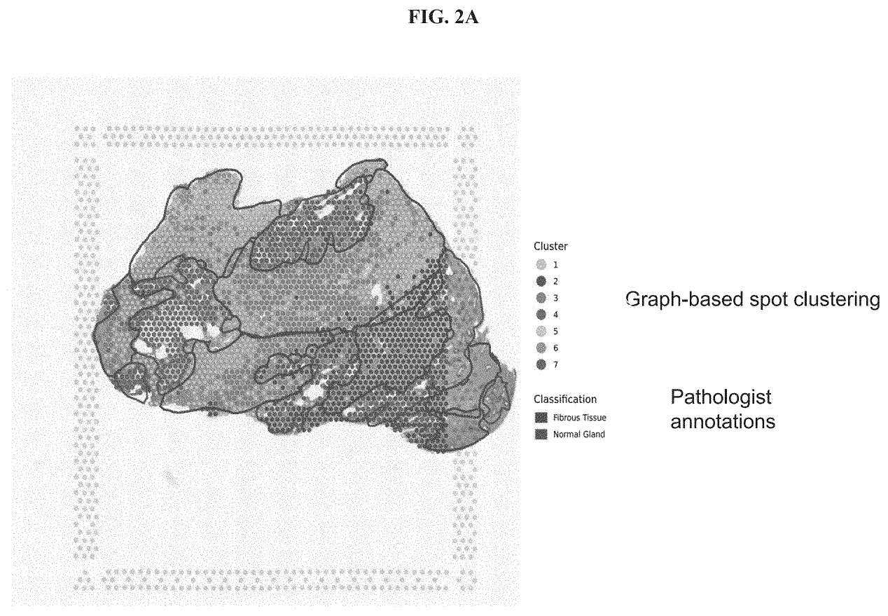

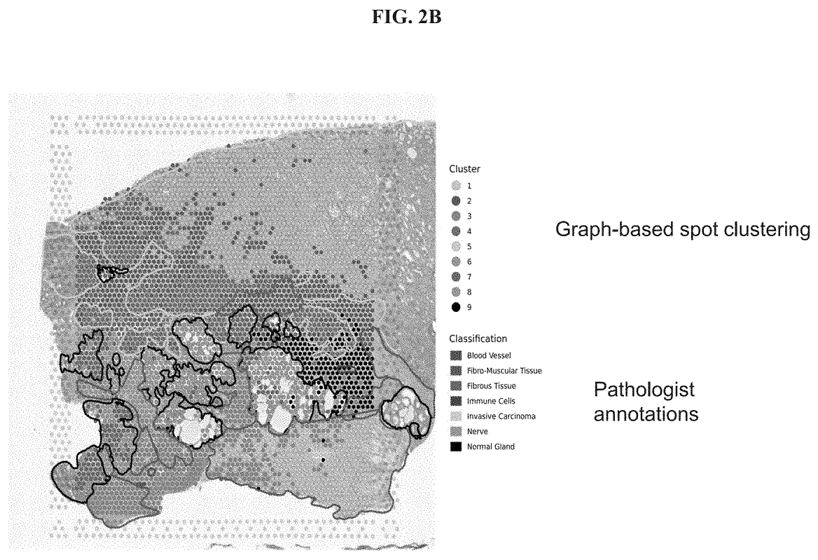

Referring now to FIG. 2A, the pathologist's annotation by H&E staining on normal prostate tissue samples reported two main classification types for tissue (or cells); fibrous tissue and normal gland. Gene expression clustering analysis correlated with the pathologist's annotation while also providing an additional layer of deeper gene expression analysis as demonstrated by the clustering results (FIG. 2A).

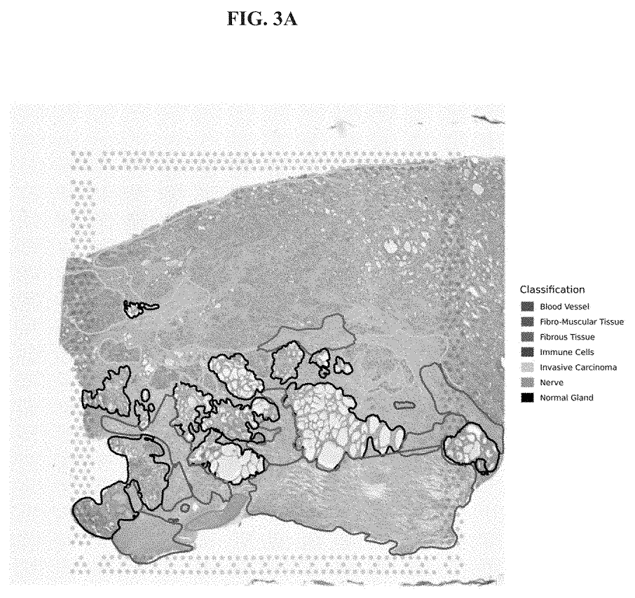

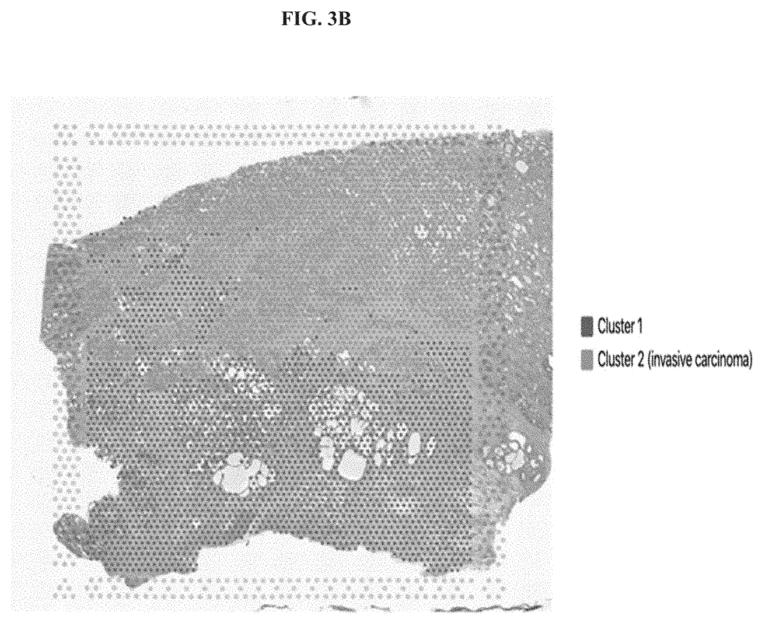

Referring to FIG. 2B, the pathologist's annotation of the prostate adenocarcinoma tissue sample shows segregation of the stage III adenocarcinoma section into seven regions with a large portion annotated as invasive carcinoma (e.g., by H&E staining). The spatial gene expression unbiased clustering (K=2) shown in FIG. 3B shows a demarcation of invasive carcinoma (Cluster 2) from benign prostate parenchyma (Cluster 1). This emarcation correlates well with the pathologist annotation. With graph-based clustering shown in FIG. 2B, nine clusters were automatically identified in the Loupe browser, with Clusters 1 and 2 showing overlap with the pathologist-annotated invasive carcinoma. Both clustering methods identified spatial patterns of gene expression that aligned well with the pathologist's annotation, however gene expression profiling is shown to be more sensitive than a pathologist annotation (e.g., two gene clusters with the invasive carcinoma were identified using gene expression profiling, whereas a pathologist identified one main region).

The same outcome is seen when comparing FIG. 3A with FIG. 3B. When analyzing the gene expression data as two clusters (K=2) as in FIG. 3B, two distinct regions are observed: an invasive carcinoma region and a benign prostate parenchyma region. The use of spatial gene expression profiling provided a more sensitive and accurate mechanism for differentiating invasive carcinoma from benign prostate parenchyma.

Tables 2 and 3 list those genes in the invasive carcinoma and benign prostate parenchyma that are over and under expressed in the invasive carcinoma region and in the benign prostate parenchyma region. These genes can be used to correlate a prostate tissue with the presence or absence, and abundance of gene markers indicative of adenocarcinoma of the prostate and invasive carcinoma therein.

TABLE 2
Adenocarcinoma Cluster 1 Top 50

| | | | Cluster 1 | | | Cluster 2 | |
|---|---|---|---|---|---|---|---|
| | | | Log2 | | | Log2 | |
| | | Cluster 1 | Fold | Cluster 1 | Cluster 2 | Fold | Cluster 2 |
| Feature ID | Gene | Average | Change | P-Value | Average | Change | P-Value |
| ENSG00000160307 | S100B | 1.023026 | 3.818614 | 2.41E−45 | 0.072209 | −3.81861 | 2.41E−45 |
| ENSG00000163810 | TGM4 | 10.82037 | 3.740633 | 2.28E−37 | 0.808934 | −3.74063 | 2.28E−37 |
| ENSG00000198732 | SMOC1 | 1.077785 | 2.98185 | 5.36E−45 | 0.136148 | −2.98185 | 5.36E−45 |
| ENSG00000012223 | LTF | 3.360912 | 2.794181 | 2.01E−31 | 0.484151 | −2.79418 | 2.01E−31 |
| ENSG00000166825 | ANPEP | 1.481713 | 2.786902 | 1.44E−40 | 0.214401 | −2.7869 | 1.44E−40 |
| ENSG00000007908 | SELE | 1.659519 | 2.769761 | 2.24E−31 | 0.24303 | −2.76976 | 2.24E−31 |
| ENSG00000167653 | PSCA | 1.635682 | 2.74513 | 1.85E−38 | 0.243666 | −2.74513 | 1.85E−38 |
| ENSG00000125144 | MT1G | 2.011265 | 2.743674 | 8.30E−40 | 0.29997 | −2.74367 | 8.30E−40 |
| ENSG00000163017 | ACTG2 | 45.64514 | 2.682949 | 2.68E−43 | 7.105447 | −2.68295 | 2.68E−43 |
| ENSG00000101443 | WFDC2 | 3.49491 | 2.641094 | 4.15E−41 | 0.55986 | −2.64109 | 4.15E−41 |
| ENSG00000184557 | SOCS3 | 4.974691 | 2.630478 | 5.61E−41 | 0.80289 | −2.63048 | 5.61E−41 |
| ENSG00000183036 | PCP4 | 6.16908 | 2.624114 | 3.19E−41 | 1.000113 | −2.62411 | 3.19E−41 |
| ENSG00000182253 | SYNM | 8.525648 | 2.623534 | 4.15E−41 | 1.38279 | −2.62353 | 4.15E−41 |
| ENSG00000099860 | GADD45B | 3.659831 | 2.584396 | 3.72E−39 | 0.609802 | −2.5844 | 3.72E−39 |
| ENSG00000041982 | TNC | 1.712989 | 2.563503 | 8.48E−38 | 0.289473 | −2.5635 | 8.48E−38 |
| ENSG00000180447 | GAS1 | 1.075208 | 2.563361 | 9.86E−36 | 0.181636 | −2.56336 | 9.86E−36 |
| ENSG00000130176 | CNN1 | 15.04583 | 2.550932 | 1.57E−39 | 2.566448 | −2.55093 | 1.57E−39 |
| ENSG00000147394 | ZNF185 | 1.337407 | 2.540034 | 6.68E−35 | 0.22967 | −2.54003 | 6.68E−35 |
| ENSG00000175084 | DES | 18.32493 | 2.464395 | 3.44E−37 | 3.319078 | −2.4644 | 3.44E−37 |
| ENSG00000004776 | HSPB6 | 3.464632 | 2.437025 | 1.02E−35 | 0.639385 | −2.43703 | 1.02E−35 |
| ENSG00000263639 | MSMB | 89.50256 | 2.430936 | 1.98E−34 | 16.59221 | −2.43094 | 1.98E−34 |
| ENSG00000065534 | MYLK | 24.64926 | 2.423879 | 3.89E−36 | 4.591804 | −2.42388 | 3.89E−36 |
| ENSG00000211896 | IGHG1 | 2.055716 | 2.414671 | 1.73E−19 | 0.385222 | −2.41467 | 1.73E−19 |
| ENSG00000128591 | FLNC | 2.81139 | 2.408926 | 1.40E−34 | 0.529004 | −2.40893 | 1.40E−34 |
| ENSG00000158859 | ADAMTS4 | 2.074398 | 2.407624 | 1.29E−32 | 0.390629 | −2.40762 | 1.29E−32 |
| ENSG00000101335 | MYL9 | 58.38143 | 2.35359 | 2.40E−34 | 11.41891 | −2.35359 | 2.40E−34 |
| ENSG00000149451 | ADAM33 | 1.068122 | 2.347376 | 6.74E−30 | 0.209629 | −2.34738 | 6.74E−30 |
| ENSG00000186081 | KRT5 | 3.483959 | 2.342599 | 1.01E−31 | 0.686464 | −2.3426 | 1.01E−31 |
| ENSG00000154330 | PGM5 | 2.037678 | 2.336795 | 2.57E−32 | 0.403035 | −2.33679 | 2.57E−32 |
| ENSG00000211957 | IGHV3-35 | 1.275562 | 2.329368 | 1.78E−07 | 0.253527 | −2.32937 | 1.78E−07 |
| ENSG00000100285 | NEFH | 4.405198 | 2.323063 | 7.69E−31 | 0.87987 | −2.32306 | 7.69E−31 |
| ENSG00000149596 | JPH2 | 1.511992 | 2.323033 | 1.41E−31 | 0.301879 | −2.32303 | 1.41E−31 |
| ENSG00000171346 | KRT15 | 6.60071 | 2.32301 | 3.88E−31 | 1.318533 | −2.32301 | 3.88E−31 |
| ENSG00000137699 | TRIM29 | 1.87018 | 2.314412 | 7.64E−31 | 0.375679 | −2.31441 | 7.64E−31 |
| ENSG00000277893 | SRD5A2 | 1.074564 | 2.312977 | 2.03E−29 | 0.215991 | −2.31298 | 2.03E−29 |
| ENSG00000152137 | HSPB8 | 2.411327 | 2.266829 | 9.64E−31 | 0.500693 | −2.26683 | 9.64E−31 |
| ENSG00000187479 | C11orf96 | 4.814279 | 2.260013 | 1.28E−30 | 1.004566 | −2.26001 | 1.28E−30 |
| ENSG00000072195 | SPEG | 1.632461 | 2.251229 | 5.74E−30 | 0.342596 | −2.25123 | 5.74E−30 |
| ENSG00000111275 | ALDH2 | 2.210329 | 2.24772 | 1.04E−30 | 0.465065 | −2.24772 | 1.04E−30 |
| ENSG00000196924 | FLNA | 35.32018 | 2.236392 | 2.65E−31 | 7.492895 | −2.23639 | 2.65E−31 |
| ENSG00000102837 | OLFM4 | 2.759208 | 2.231375 | 1.97E−22 | 0.587217 | −2.23137 | 1.97E−22 |
| ENSG00000278857 | IGKV1D-12 | 2.365587 | 2.227348 | 3.60E−13 | 0.504828 | −2.22735 | 3.60E−13 |
| ENSG00000149591 | TAGLN | 48.31802 | 2.217686 | 7.38E−31 | 10.38413 | −2.21769 | 7.38E−31 |
| ENSG00000198467 | TPM2 | 31.69384 | 2.216972 | 8.98E−31 | 6.814702 | −2.21697 | 8.98E−31 |
| ENSG00000109846 | CRYAB | 2.895139 | 2.214332 | 1.97E−29 | 0.62348 | −2.21433 | 1.97E−29 |
| ENSG00000183963 | SMTN | 8.271825 | 2.212799 | 2.89E−30 | 1.783599 | −2.2128 | 2.89E−30 |
| ENSG00000145936 | KCNMB1 | 1.749066 | 2.210549 | 9.13E−29 | 0.377587 | −2.21055 | 9.13E−29 |
| ENSG00000109099 | PMP22 | 1.012074 | 2.199274 | 1.79E−25 | 0.220127 | −2.19927 | 1.79E−25 |
| ENSG00000107796 | ACTA2 | 51.24344 | 2.182673 | 5.05E−30 | 11.2834 | −2.18267 | 5.05E−30 |
| ENSG00000172403 | SYNPO2 | 9.903642 | 2.176983 | 1.83E−29 | 2.189179 | −2.17698 | 1.83E−29 |

TABLE 3
Adenocarcinoma Cluster 2 Top 50

| | | | Cluster 1 | | | Cluster 2 | |
|---|---|---|---|---|---|---|---|
| | | | Log2 | | | Log2 | |
| | | Cluster 1 | Fold | Cluster 1 | Cluster 2 | Fold | Cluster 2 |
| Feature ID | Gene | Average | Change | P-Value | Average | Change | P-Value |
| ENSG00000153292 | ADGRF1 | 0.957316 | −2.46632 | 8.46E−27 | 5.291947 | 2.466319 | 8.46E−27 |
| ENSG00000144339 | TMEFF2 | 6.926043 | −2.30235 | 1.92E−24 | 34.15526 | 2.302354 | 1.92E−24 |
| ENSG00000096006 | CRISP3 | 1.758085 | −2.29084 | 3.72E−24 | 8.603072 | 2.290842 | 3.72E−24 |
| ENSG00000197674 | OR51C1P | 4.867106 | −2.26009 | 1.05E−23 | 23.30957 | 2.260085 | 1.05E−23 |
| ENSG00000166006 | KCNC2 | 0.869701 | −2.2591 | 1.98E−23 | 4.164593 | 2.259096 | 1.98E−23 |
| ENSG00000146070 | PLA2G7 | 1.462386 | −2.23623 | 3.39E−23 | 6.890728 | 2.236233 | 3.39E−23 |
| ENSG00000159674 | SPON2 | 5.685269 | −2.23263 | 7.45E−23 | 26.71422 | 2.232631 | 7.45E−23 |
| ENSG00000162545 | CAMK2N1 | 3.652745 | −2.22045 | 6.98E−23 | 17.02037 | 2.220452 | 6.98E−23 |
| ENSG00000122585 | NPY | 3.87629 | −2.21066 | 7.94E−23 | 17.93969 | 2.210662 | 7.94E−23 |
| ENSG00000080709 | KCNN2 | 0.758251 | −2.21008 | 1.60E−22 | 3.509939 | 2.210075 | 1.60E−22 |
| ENSG00000167332 | OR51E2 | 6.710872 | −2.19816 | 1.04E−22 | 30.78847 | 2.198165 | 1.04E−22 |
| ENSG00000144891 | AGTR1 | 0.505071 | −2.19438 | 1.05E−21 | 2.313557 | 2.194384 | 1.05E−21 |
| ENSG00000198785 | GRIN3A | 0.521176 | −2.16552 | 2.61E−21 | 2.339959 | 2.165524 | 2.61E−21 |
| ENSG00000242110 | AMACR | 5.86243 | −2.15409 | 6.23E−22 | 26.08692 | 2.154085 | 6.23E−22 |
| ENSG00000131773 | KHDRBS3 | 0.694472 | −2.14547 | 2.03E−21 | 3.074139 | 2.145475 | 2.03E−21 |
| ENSG00000183960 | KCNH8 | 0.433562 | −2.14158 | 8.16E−21 | 1.914975 | 2.141582 | 8.16E−21 |
| ENSG00000132932 | ATP8A2 | 0.266708 | −2.13952 | 2.78E−20 | 1.177296 | 2.139522 | 2.78E−20 |
| ENSG00000196932 | TMEM26 | 0.295054 | −2.13015 | 2.78E−20 | 1.293721 | 2.130153 | 2.78E−20 |
| ENSG00000187398 | LUZP2 | 0.44387 | −2.09474 | 4.15E−20 | 1.897797 | 2.094737 | 4.15E−20 |
| ENSG00000105707 | HPN | 0.794971 | −2.08075 | 2.05E−20 | 3.364248 | 2.080747 | 2.05E−20 |
| ENSG00000188257 | PLA2G2A | 8.937951 | −2.07668 | 2.20E−20 | 37.69351 | 2.076679 | 2.20E−20 |
| ENSG00000139219 | COL2A1 | 0.307294 | −2.07199 | 2.45E−19 | 1.294039 | 2.071992 | 2.45E−19 |
| ENSG00000144406 | UNC80 | 0.35239 | −2.06303 | 2.54E−19 | 1.474403 | 2.063033 | 2.54E−19 |
| ENSG00000137976 | DNASE2B | 0.482523 | −2.04452 | 2.51E−19 | 1.992274 | 2.044522 | 2.51E−19 |
| ENSG00000157554 | ERG | 0.916085 | −2.00378 | 3.17E−19 | 3.675034 | 2.003785 | 3.17E−19 |
| ENSG00000197757 | HOXC6 | 0.258978 | −1.96257 | 1.92E−17 | 1.011247 | 1.962575 | 1.92E−17 |
| ENSG00000152377 | SPOCK1 | 2.503451 | −1.94327 | 1.79E−18 | 9.626724 | 1.943274 | 1.79E−18 |
| ENSG00000162482 | AKR7A3 | 0.300852 | −1.93461 | 3.18E−17 | 1.151848 | 1.934606 | 3.18E−17 |
| ENSG00000175928 | LRRN1 | 0.267997 | −1.90375 | 1.31E−16 | 1.004566 | 1.903749 | 1.31E−16 |
| ENSG00000135052 | GOLM1 | 7.282942 | −1.87142 | 2.01E−17 | 26.64105 | 1.871418 | 2.01E−17 |
| ENSG00000169562 | GJB1 | 0.409082 | −1.85253 | 2.68E−16 | 1.478857 | 1.852532 | 2.68E−16 |
| ENSG00000127418 | FGFRL1 | 1.150582 | −1.81695 | 2.47E−16 | 4.05453 | 1.816946 | 2.47E−16 |
| ENSG00000112964 | GHR | 0.287968 | −1.81076 | 2.59E−15 | 1.011883 | 1.810763 | 2.59E−15 |
| ENSG00000005187 | ACSM3 | 0.517955 | −1.79436 | 1.44E−15 | 1.797913 | 1.794356 | 1.44E−15 |
| ENSG00000131844 | MCCC2 | 3.114819 | −1.7892 | 3.94E−16 | 10.76394 | 1.789201 | 3.94E−16 |
| ENSG00000211695 | TRGV9 | 0.901912 | −1.78666 | 7.73E−16 | 3.112629 | 1.786662 | 7.73E−16 |
| ENSG00000144331 | ZNF385B | 0.479302 | −1.72671 | 1.46E−14 | 1.587648 | 1.726705 | 1.46E−14 |
| ENSG00000146233 | CYP39A1 | 0.726039 | −1.69362 | 1.95E−14 | 2.349502 | 1.693621 | 1.95E−14 |
| ENSG00000166562 | SEC11C | 5.301312 | −1.68997 | 1.01E−14 | 17.10085 | 1.689969 | 1.01E−14 |
| ENSG00000246705 | H2AFJ | 5.695576 | −1.65966 | 2.86E−14 | 17.99058 | 1.659661 | 2.86E−14 |
| ENSG00000196586 | MYO6 | 1.994515 | −1.62579 | 1.06E−13 | 6.154958 | 1.62579 | 1.06E−13 |
| ENSG00000173890 | GPR160 | 0.882586 | −1.61633 | 2.15E−13 | 2.706731 | 1.61633 | 2.15E−13 |
| ENSG00000138696 | BMPR1B | 1.129967 | −1.59867 | 3.11E−13 | 3.422779 | 1.598669 | 3.11E−13 |
| ENSG00000143797 | MBOAT2 | 1.287802 | −1.58718 | 4.35E−13 | 3.869712 | 1.587183 | 4.35E−13 |
| ENSG00000115339 | GALNT3 | 1.060391 | −1.57203 | 7.73E−13 | 3.153346 | 1.572027 | 7.73E−13 |
| ENSG00000196353 | CPNE4 | 1.18537 | −1.57062 | 7.73E−13 | 3.52139 | 1.570624 | 7.73E−13 |
| ENSG00000078124 | ACER3 | 0.421966 | −1.57029 | 2.66E−12 | 1.254277 | 1.570292 | 2.66E−12 |
| ENSG00000054690 | PLEKHH1 | 1.176996 | −1.56354 | 9.74E−13 | 3.479401 | 1.563543 | 9.74E−13 |
| ENSG00000188959 | C9orf152 | 0.537282 | −1.5587 | 2.60E−12 | 1.58383 | 1.558697 | 2.60E−12 |
| ENSG00000160781 | PAQR6 | 0.455466 | −1.54684 | 4.85E−12 | 1.331894 | 1.54684 | 4.85E−12 |

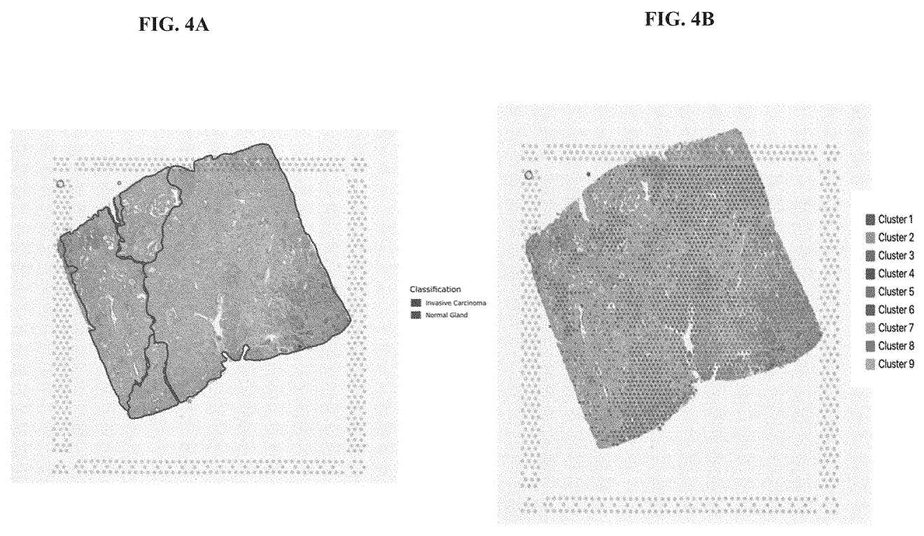

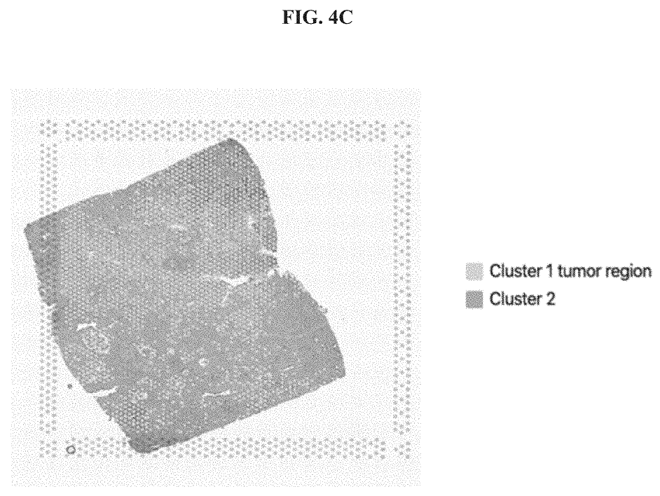

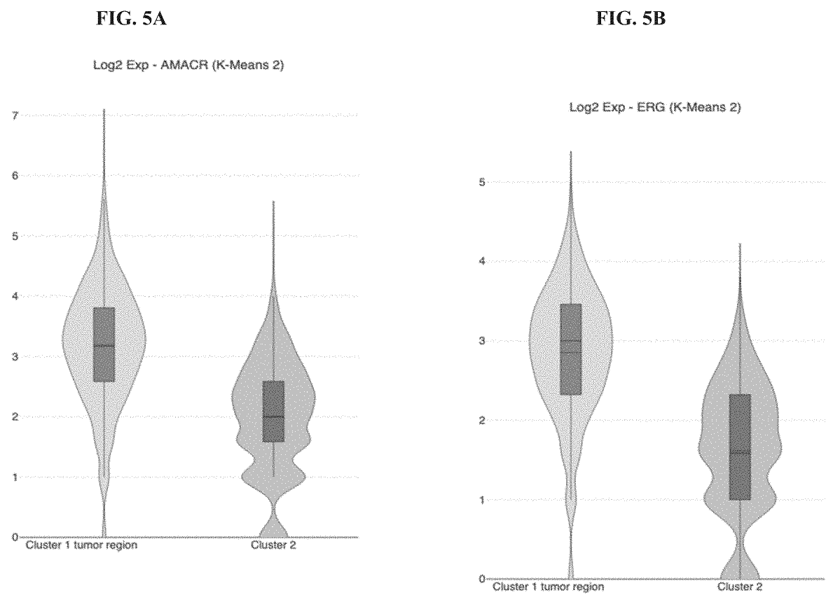

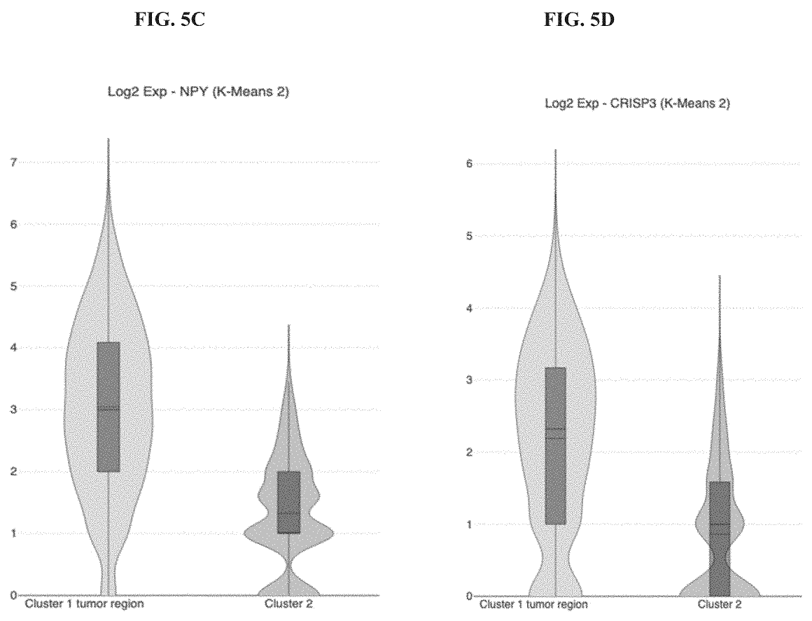

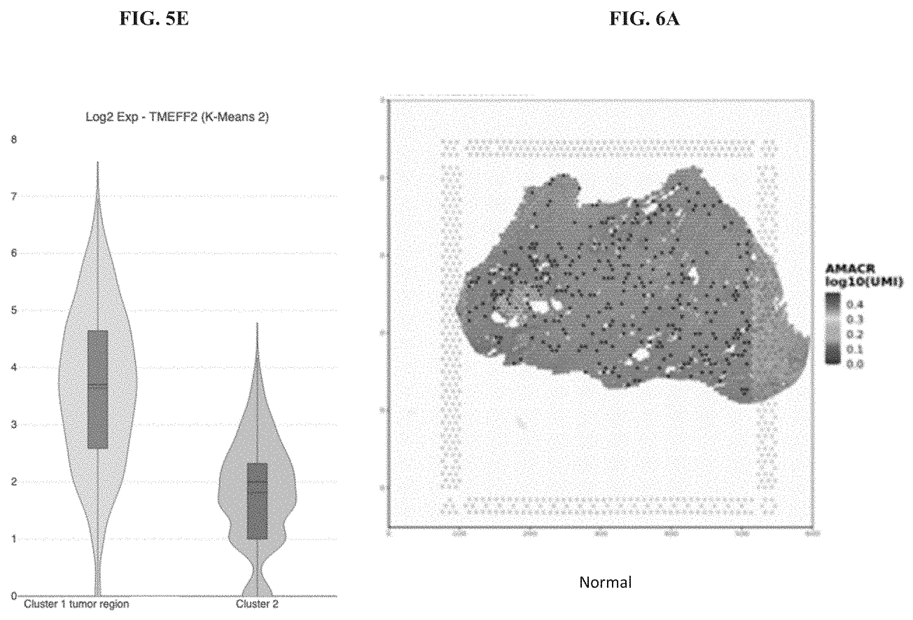

An acinar cell carcinoma prostate cancer was analyzed similar to the adenocarcinoma sample described above. H&E staining was performed on an acinar cell carcinoma prostate sample, followed by a pathologist's annotation of the stained sample. This stained and annotated sample was compared to spatial gene expression clustering data, which included whole transcriptome gene expression analysis graph based clustering (FIG. 4B) and K-means K=2 clustering (FIG. 4C). While the pathologist annotated the acinar cell carcinoma as being almost wholly invasive carcinoma (FIG. 4A), the gene expression clustering analysis using either graph based (FIG. 4B) or K-means (FIG. 4C) clustering (demonstrated that the acinar cell carcinoma was not totally invasive. For instance, several prostate cancer markers were observed in the gene expression clustering analysis of the acinar cell carcinoma sample, as presented in FIG. 5A-FIG. 5F. For example, the gene expression profiles for several known prostate cancer biomarkers: AMACR (FIG. 5B), ERG (FIG. 5C), NPY (FIG. 5D), CRISP3 (FIG. 5E), and TMEFF2 (FIG. 5F) were found to be highly expressed in Cluster 1 (tumor region) as compared to Cluster 2 (non-tumor region).

Tables 4 and 5 lists those genes in the acinar cell carcinoma that are over and under expressed, respectively, and that could be used for determining the presence or absence, and abundance of genes correlated with acinar cell carcinoma in prostate tissue samples.

TABLE 4
Acinar cell carcinoma over expressed genes

| | | | Cluster 1 | | | Cluster 2 | |
|---|---|---|---|---|---|---|---|
| | | | Log2 | | | Log2 | |
| | | Cluster 1 | Fold | Cluster 1 | Cluster 2 | Fold | Cluster 2 |
| Feature ID | Gene | Average | Change | P-Value | Average | Change | P-Value |
| ENSG00000122585 | NPY | 9.101128 | 1.826843 | 7.55E−12 | 2.565825 | −1.82684 | 7.55E−12 |
| ENSG00000144339 | TMEFF2 | 14.03574 | 1.784726 | 1.78E−11 | 4.0747 | −1.78473 | 1.78E−11 |
| ENSG00000180785 | OR51E1 | 2.674987 | 1.826973 | 3.01E−11 | 0.753515 | −1.82697 | 3.01E−11 |
| ENSG00000068078 | FGFR3 | 2.428625 | 1.774848 | 5.12E−10 | 0.709245 | −1.77485 | 5.12E−10 |
| ENSG00000159674 | SPON2 | 32.57876 | 1.548936 | 5.44E−09 | 11.13856 | −1.54894 | 5.44E−09 |
| ENSG00000171126 | KCNG3 | 1.031958 | 1.574589 | 2.02E−08 | 0.345861 | −1.57459 | 2.02E−08 |
| ENSG00000096006 | CRISP3 | 4.355316 | 1.442037 | 1.19E−07 | 1.602949 | −1.44204 | 1.19E−07 |
| ENSG00000197674 | OR51C1P | 2.6119 | 1.420017 | 1.89E−07 | 0.975788 | −1.42002 | 1.89E−07 |
| ENSG00000081181 | ARG2 | 5.29886 | 1.303462 | 1.19E−06 | 2.147103 | −1.30346 | 1.19E−06 |
| ENSG00000143753 | DEGS1 | 9.48886 | 1.281649 | 1.81E−06 | 3.904076 | −1.28165 | 1.81E−06 |
| ENSG00000167332 | OR51E2 | 5.750601 | 1.266015 | 2.83E−06 | 2.391512 | −1.26602 | 2.83E−06 |
| ENSG00000211688 | TRGJP2 | 2.541906 | 1.254396 | 3.88E−06 | 1.065251 | −1.2544 | 3.88E−06 |
| ENSG00000196353 | CPNE4 | 6.922549 | 1.214999 | 5.73E−06 | 2.982703 | −1.215 | 5.73E−06 |
| ENSG00000135052 | GOLM1 | 33.73229 | 1.200312 | 7.06E−06 | 14.68571 | −1.20031 | 7.06E−06 |
| ENSG00000072042 | RDH11 | 21.70336 | 1.180952 | 9.74E−06 | 9.576191 | −1.18095 | 9.74E−06 |
| ENSG00000166743 | ACSM1 | 3.043379 | 1.138295 | 2.77E−05 | 1.382521 | −1.1383 | 2.77E−05 |
| ENSG00000054690 | PLEKHH1 | 3.020815 | 1.105607 | 5.36E−05 | 1.403733 | −1.10561 | 5.36E−05 |
| ENSG00000104154 | SLC30A4 | 8.277311 | 1.089365 | 5.82E−05 | 3.891164 | −1.08936 | 5.82E−05 |
| ENSG00000211689 | TRGC1 | 8.146992 | 1.088519 | 5.82E−05 | 3.832137 | −1.08852 | 5.82E−05 |
| ENSG00000135414 | GDF11 | 1.275558 | 1.117386 | 7.44E−05 | 0.587502 | −1.11739 | 7.44E−05 |
| ENSG00000131773 | KHDRBS3 | 1.533432 | 1.098576 | 9.55E−05 | 0.715701 | −1.09858 | 9.55E−05 |
| ENSG00000152377 | SPOCK1 | 5.722511 | 1.060755 | 9.57E−05 | 2.743828 | −1.06076 | 9.57E−05 |
| ENSG00000130513 | GDF15 | 6.760456 | 1.048392 | 0.000133 | 3.269537 | −1.04839 | 0.000133 |
| ENSG00000127418 | FGFRL1 | 2.861946 | 1.03909 | 0.000178 | 1.392666 | −1.03909 | 0.000178 |
| ENSG00000211695 | TRGV9 | 2.262849 | 1.031563 | 0.000184 | 1.106754 | −1.03156 | 0.000184 |
| ENSG00000175928 | LRRN1 | 1.739732 | 1.032209 | 0.000204 | 0.850356 | −1.03221 | 0.000204 |
| ENSG00000146070 | PLA2G7 | 6.645794 | 1.013175 | 0.000205 | 3.293516 | −1.01317 | 0.000205 |
| ENSG00000158164 | TMSB15A | 1.902285 | 1.01971 | 0.000255 | 0.937974 | −1.01971 | 0.000255 |
| ENSG00000080709 | KCNN2 | 2.867472 | 1.006039 | 0.000262 | 1.427713 | −1.00604 | 0.000262 |
| ENSG00000142515 | KLK3 | 269.0948 | 0.984014 | 0.00029 | 136.1086 | −0.98401 | 0.00029 |
| ENSG00000186205 | MARC1 | 1.299964 | 1.012983 | 0.000364 | 0.643762 | −1.01298 | 0.000364 |
| ENSG00000196090 | PTPRT | 1.220759 | 1.033851 | 0.000364 | 0.595803 | −1.03385 | 0.000364 |
| ENSG00000181751 | C5orf30 | 1.250231 | 1.013558 | 0.000367 | 0.61886 | −1.01356 | 0.000367 |
| ENSG00000158270 | COLEC12 | 2.439677 | 0.968166 | 0.000507 | 1.246943 | −0.96817 | 0.000507 |
| ENSG00000105707 | HPN | 3.904036 | 0.955529 | 0.000526 | 2.01337 | −0.95553 | 0.000526 |
| ENSG00000196586 | MYO6 | 6.119454 | 0.939271 | 0.000692 | 3.192064 | −0.93927 | 0.000692 |
| ENSG00000153292 | ADGRF1 | 3.72997 | 0.936732 | 0.000814 | 1.94881 | −0.93673 | 0.000814 |
| ENSG00000170035 | UBE2E3 | 8.000096 | 0.924956 | 0.000822 | 4.214889 | −0.92496 | 0.000822 |
| ENSG00000113924 | HGD | 1.186223 | 0.963762 | 0.000829 | 0.607793 | −0.96376 | 0.000829 |
| ENSG00000109685 | NSD2 | 2.371985 | 0.928645 | 0.000948 | 1.246021 | −0.92864 | 0.000948 |
| ENSG00000139116 | KIF21A | 2.765704 | 0.920769 | 0.000957 | 1.460916 | −0.92077 | 0.000957 |
| ENSG00000180573 | HIST1H2AC | 3.847856 | 0.916241 | 0.000992 | 2.039195 | −0.91624 | 0.000992 |
| ENSG00000168924 | LETM1 | 2.089704 | 0.923971 | 0.001017 | 1.101221 | −0.92397 | 0.001017 |
| ENSG00000049089 | COL9A2 | 4.455242 | 0.911811 | 0.001021 | 2.368454 | −0.91181 | 0.001021 |
| ENSG00000188848 | BEND4 | 1.078007 | 0.94658 | 0.001021 | 0.558911 | −0.94658 | 0.001021 |
| ENSG00000169562 | GJB1 | 1.599282 | 0.921786 | 0.00104 | 0.8439 | −0.92179 | 0.00104 |
| ENSG00000112379 | ARFGEF3 | 4.068431 | 0.905661 | 0.001079 | 2.172005 | −0.90566 | 0.001079 |
| ENSG00000159182 | PRAC1 | 9.189542 | 0.900591 | 0.001089 | 4.924134 | −0.90059 | 0.001089 |
| ENSG00000117525 | F3 | 9.141651 | 0.892243 | 0.001364 | 4.926901 | −0.89224 | 0.001364 |
| ENSG00000203727 | SAMD5 | 2.154173 | 0.895971 | 0.001433 | 1.157481 | −0.89597 | 0.001433 |

TABLE 5
Acinar Cell Carcinoma under expressed genes

| | | | Cluster 1 | | | Cluster 2 | |
|---|---|---|---|---|---|---|---|
| | | | Log2 | | | Log2 | |
| | | Cluster 1 | Fold | Cluster 1 | Cluster 2 | Fold | Cluster 2 |
| Feature ID | Gene | Average | Change | P-Value | Average | Change | P-Value |
| ENSG00000211895 | IGHA1 | 81.44505 | −1.87743 | 3.19E−15 | 299.3844 | 1.877431 | 3.19E−15 |
| ENSG00000132465 | JCHAIN | 26.41187 | −1.82767 | 8.18E−15 | 93.7965 | 1.827671 | 8.18E−15 |
| ENSG00000105369 | CD79A | 0.422269 | −1.9064 | 8.18E−15 | 1.584503 | 1.906396 | 8.18E−15 |
| ENSG00000211900 | IGHJ6 | 0.493645 | −1.94937 | 1.41E−14 | 1.908229 | 1.949373 | 1.41E−14 |
| ENSG00000211592 | IGKC | 161.4096 | −1.78435 | 1.85E−14 | 556.2556 | 1.784354 | 1.85E−14 |
| ENSG00000134339 | SAA2 | 2.14174 | −1.80218 | 4.55E−13 | 7.473358 | 1.802176 | 4.55E−13 |
| ENSG00000172724 | CCL19 | 0.476607 | −1.94676 | 6.66E−13 | 1.839057 | 1.946757 | 6.66E−13 |
| ENSG00000211675 | IGLC1 | 24.26553 | −1.76271 | 8.51E−13 | 82.38033 | 1.762715 | 8.51E−13 |
| ENSG00000173432 | SAA1 | 0.85467 | −1.76606 | 1.19E−12 | 2.908919 | 1.766059 | 1.19E−12 |
| ENSG00000211893 | IGHG2 | 3.29711 | −2.13649 | 1.29E−12 | 14.50494 | 2.136495 | 1.29E−12 |
| ENSG00000110777 | POU2AF1 | 0.376681 | −1.71814 | 1.90E−12 | 1.240487 | 1.718136 | 1.90E−12 |
| ENSG00000099958 | DERL3 | 0.41306 | −1.7134 | 2.16E−12 | 1.355774 | 1.713402 | 2.16E−12 |
| ENSG00000163220 | S100A9 | 0.559956 | −1.86211 | 2.45E−12 | 2.03735 | 1.86211 | 2.45E−12 |
| ENSG00000211896 | IGHG1 | 53.76407 | −1.65855 | 3.33E−12 | 169.8121 | 1.658555 | 3.33E−12 |
| ENSG00000227507 | LTB | 0.407534 | −1.73281 | 4.77E−12 | 1.355774 | 1.732811 | 4.77E−12 |
| ENSG00000125730 | C3 | 1.021367 | −1.64517 | 4.77E−12 | 3.196675 | 1.645171 | 4.77E−12 |
| ENSG00000170476 | MZB1 | 1.112544 | −1.58576 | 2.05E−11 | 3.341476 | 1.585758 | 2.05E−11 |
| ENSG00000211598 | IGKV4-1 | 3.653529 | −1.70882 | 4.93E−11 | 11.94926 | 1.708822 | 4.93E−11 |
| ENSG00000118849 | RARRES1 | 0.527261 | −1.59055 | 5.26E−11 | 1.589115 | 1.590545 | 5.26E−11 |
| ENSG00000012223 | LTF | 19.44097 | −1.61646 | 5.26E−11 | 59.63838 | 1.616463 | 5.26E−11 |
| ENSG00000211897 | IGHG3 | 8.156202 | −1.69154 | 5.48E−10 | 26.35735 | 1.691539 | 5.48E−10 |
| ENSG00000211904 | IGHJ2 | 0.454043 | −1.74186 | 1.48E−09 | 1.519943 | 1.741862 | 1.48E−09 |
| ENSG00000211673 | IGLV3−1 | 1.845645 | −1.97084 | 1.52E−09 | 7.239095 | 1.970843 | 1.52E−09 |
| ENSG00000090382 | LYZ | 2.097533 | −1.43796 | 1.94E−09 | 5.68595 | 1.43796 | 1.94E−09 |
| ENSG00000130592 | LSP1 | 0.91085 | −1.41223 | 4.03E−09 | 2.425636 | 1.412232 | 4.03E−09 |
| ENSG00000102837 | OLFM4 | 2.155094 | −1.4779 | 1.24E−08 | 6.005987 | 1.477898 | 1.24E−08 |
| ENSG00000275395 | FCGBP | 0.541076 | −1.72579 | 1.32E−08 | 1.791097 | 1.72579 | 1.32E−08 |
| ENSG00000243649 | CFB | 3.713393 | −1.34544 | 3.90E−08 | 9.440614 | 1.345439 | 3.90E−08 |
| ENSG00000162366 | PDZK1IP1 | 0.931111 | −1.35673 | 6.68E−08 | 2.385978 | 1.356734 | 6.68E−08 |
| ENSG00000135480 | KRT7 | 0.637779 | −1.38166 | 7.41E−08 | 1.662898 | 1.381665 | 7.41E−08 |
| ENSG00000102879 | CORO1A | 0.515749 | −1.32512 | 1.19E−07 | 1.293058 | 1.325121 | 1.19E−07 |
| ENSG00000168685 | IL7R | 0.533248 | −1.33744 | 1.31E−07 | 1.348396 | 1.337442 | 1.31E−07 |
| ENSG00000057657 | PRDM1 | 0.55397 | −1.28643 | 2.25E−07 | 1.352085 | 1.286426 | 2.25E−07 |
| ENSG00000051523 | CYBA | 1.213852 | −1.24958 | 2.47E−07 | 2.887706 | 1.249579 | 2.47E−07 |
| ENSG00000211899 | IGHM | 0.850986 | −1.56672 | 6.82E−07 | 2.522477 | 1.566716 | 6.82E−07 |
| ENSG00000148346 | LCN2 | 4.616414 | −1.2936 | 6.89E−07 | 11.3221 | 1.293604 | 6.89E−07 |
| ENSG00000132386 | SERPINF1 | 1.555996 | −1.20858 | 6.95E−07 | 3.597874 | 1.208583 | 6.95E−07 |
| ENSG00000180353 | HCLS1 | 0.446215 | −1.24068 | 8.17E−07 | 1.055106 | 1.240683 | 8.17E−07 |
| ENSG00000143119 | CD53 | 0.425953 | −1.23531 | 1.12E−06 | 1.003457 | 1.235312 | 1.12E−06 |
| ENSG00000081237 | PTPRC | 0.785596 | −1.20959 | 1.12E−06 | 1.817844 | 1.209589 | 1.12E−06 |
| ENSG00000107317 | PTGDS | 4.792321 | −1.16452 | 1.54E−06 | 10.74751 | 1.164525 | 1.54E−06 |
| ENSG00000166741 | NNMT | 1.124056 | −1.18841 | 1.58E−06 | 2.563058 | 1.188414 | 1.58E−06 |
| ENSG00000141753 | IGFBP4 | 1.730983 | −1.1583 | 2.00E−06 | 3.865339 | 1.158299 | 2.00E−06 |
| ENSG00000101443 | WFDC2 | 1.939585 | −1.18506 | 2.00E−06 | 4.41226 | 1.185064 | 2.00E−06 |
| ENSG00000047457 | CP | 0.829343 | −1.20003 | 2.24E−06 | 1.906384 | 1.20003 | 2.24E−06 |
| ENSG00000160255 | ITGB2 | 0.544299 | −1.18976 | 2.29E−06 | 1.242332 | 1.189763 | 2.29E−06 |
| ENSG00000159403 | C1R | 6.045775 | −1.12111 | 3.50E−06 | 13.15654 | 1.121106 | 3.50E−06 |
| ENSG00000120885 | CLU | 4.1527 | −1.12826 | 3.63E−06 | 9.081841 | 1.128256 | 3.63E−06 |
| ENSG00000171346 | KRT15 | 1.200498 | −1.20293 | 4.09E−06 | 2.765041 | 1.20293 | 4.09E−06 |
| ENSG00000115641 | FHL2 | 0.568245 | −1.14473 | 5.11E−06 | 1.257088 | 1.144725 | 5.11E−06 |

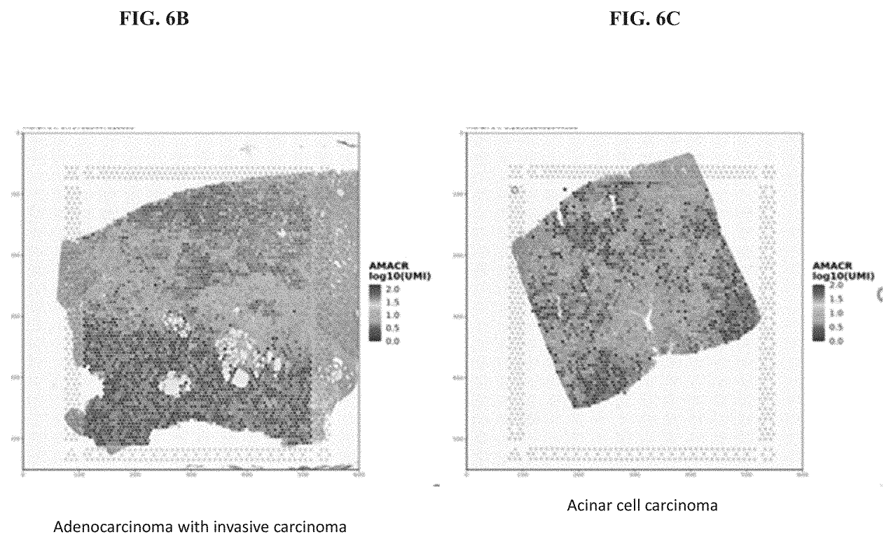

Alpha-methylacyl-CoA, or AMACR, gene expression in a normal, non-cancerous prostate tissue sample was determined and compared to the AMACR gene expression determined for the adenocarcinoma prostate carcinoma sample and acinar cell carcinoma prostate tissue sample described above. AMACR is a gene known to be overexpressed and highly correlated with prostate cancer risk and diagnosis. As presented in FIG. 6A, the normal, non-cancerous prostate cancer sample had a low level of AMACR gene expression. AMACR was highly expressed in areas of the invasive carcinoma region of the adenocarcinoma prostate carcinoma sample (FIG. 6B), indicating a cancerous region of the sample, however, AMACR was not expressed throughout the acinar carcinoma prostate sample (FIG. 6C).

Furthermore, when comparing the invasive region of the adenocarcinoma sample as determined by the pathologist (FIG. 3A) as compared to the AMACR gene expression level, it was shown that the invasive carcinoma of the adenocarcinoma sample, while showing high expression of AMACR in a large part of the region, was not found throughout the region that was determined by a pathologist as being wholly invasive.

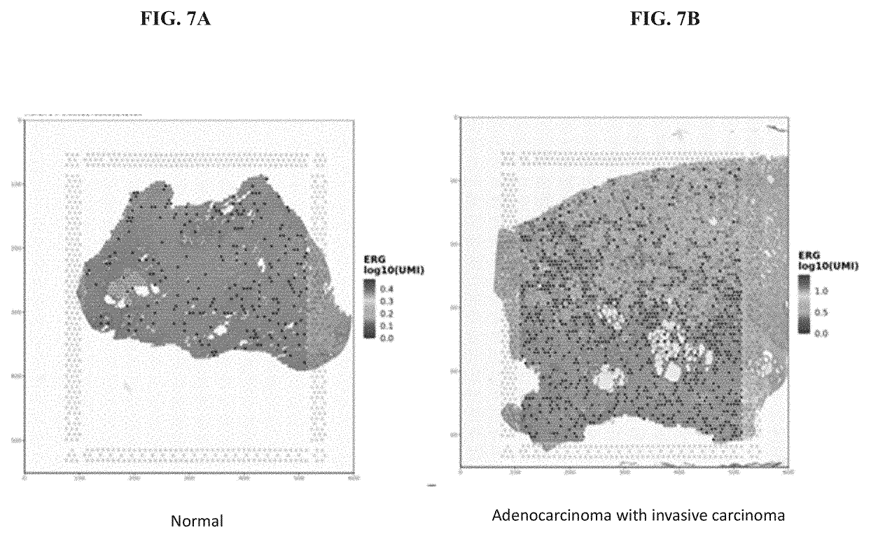

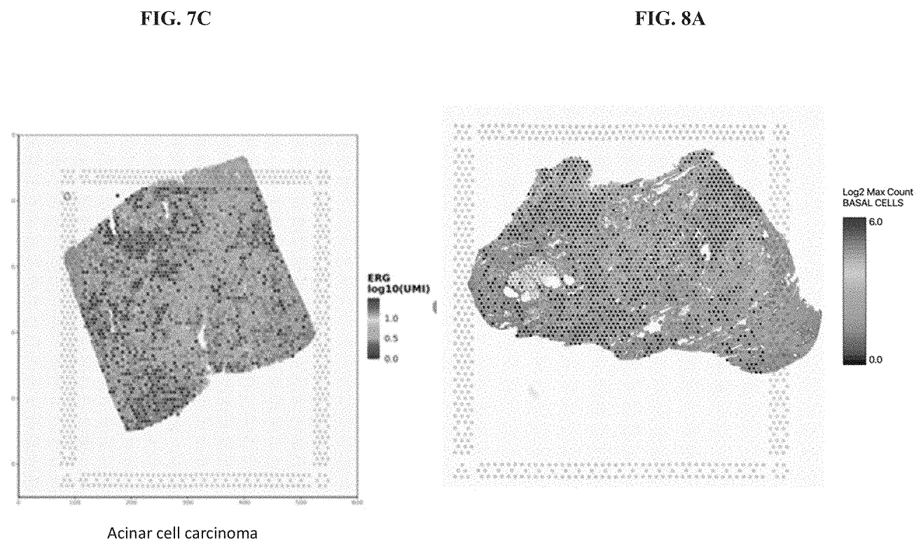

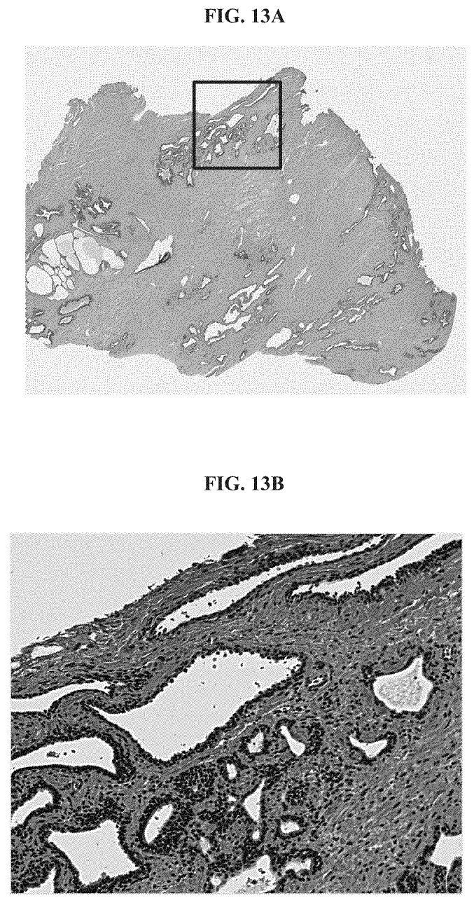

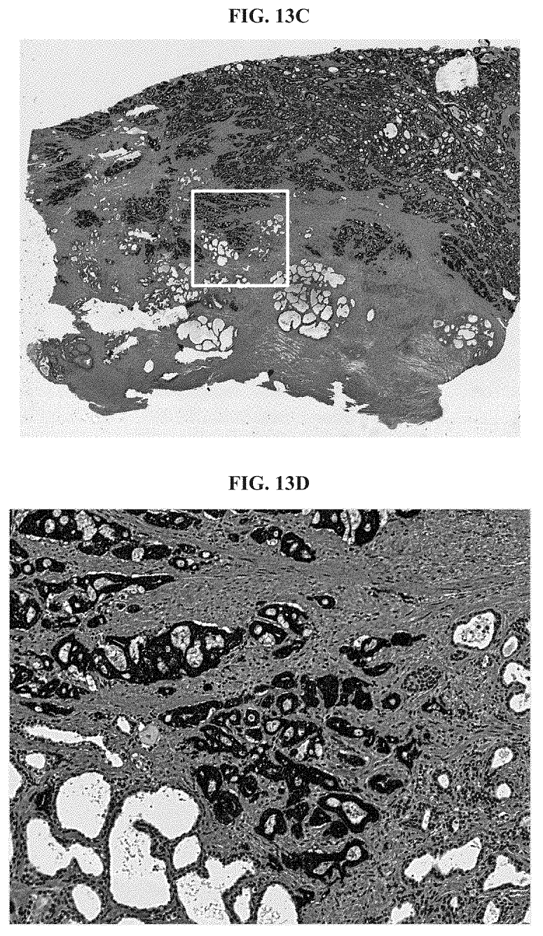

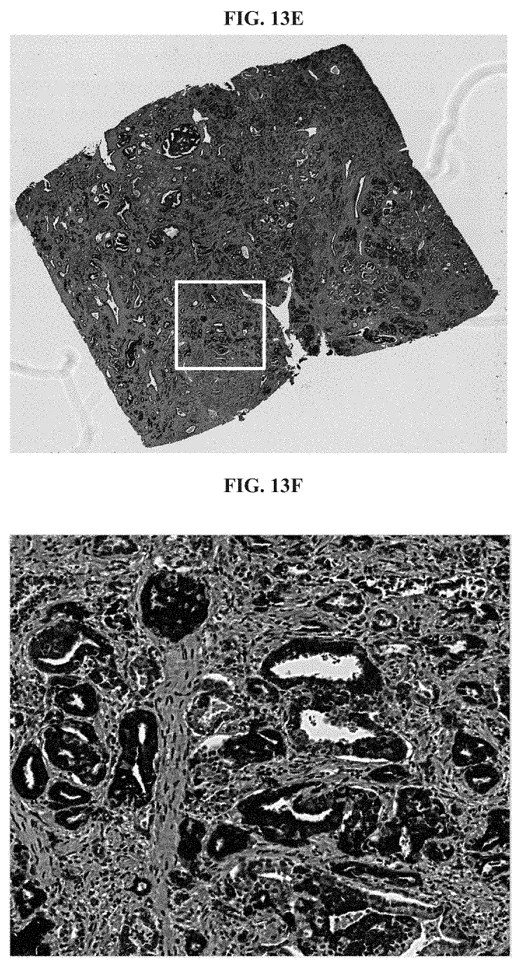

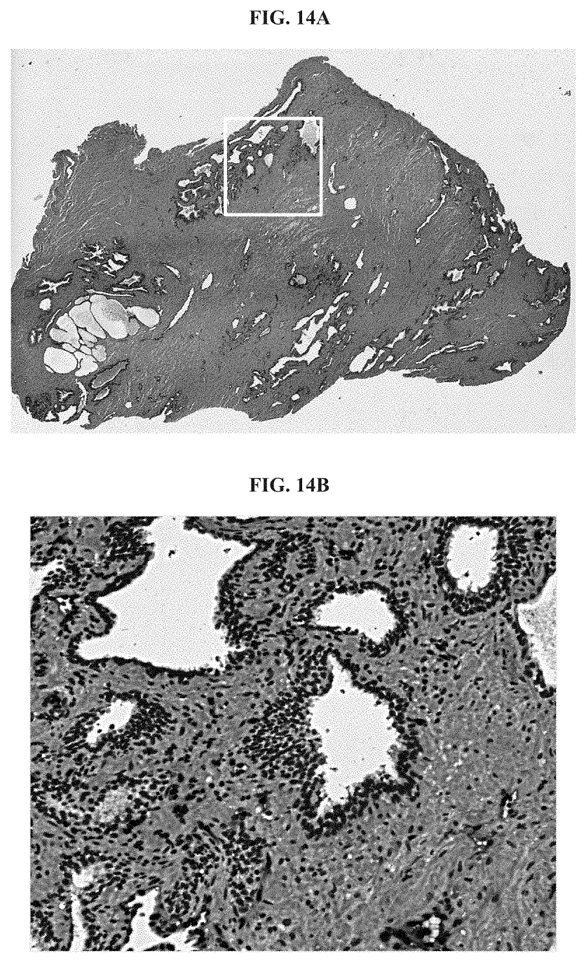

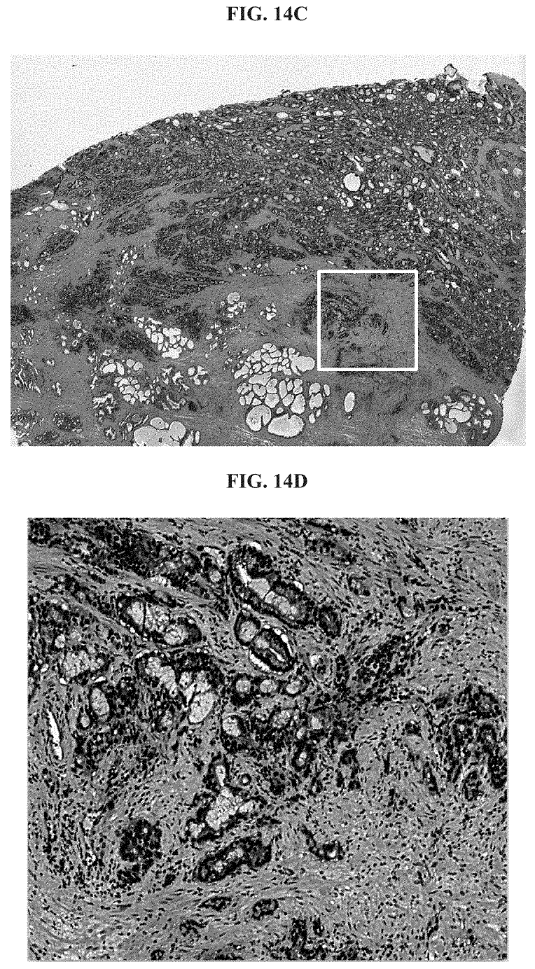

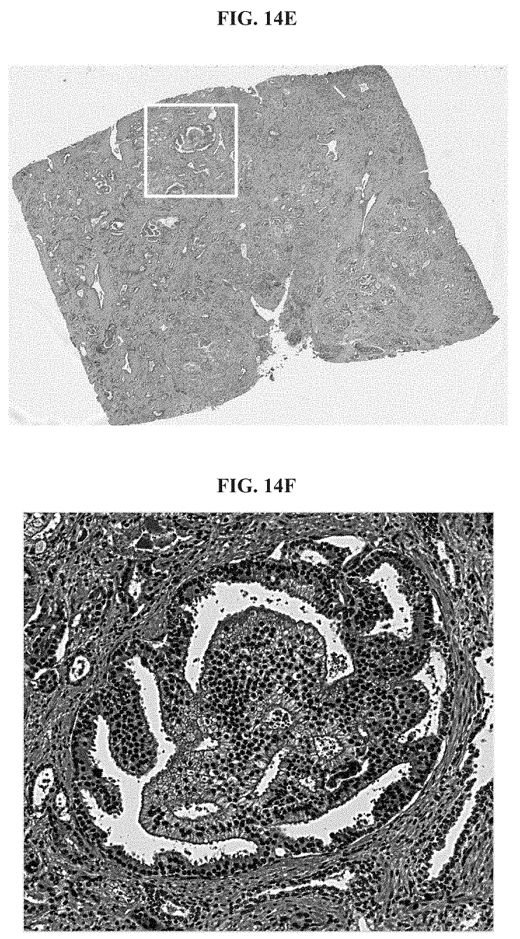

Moreover, results related to ERG gene expression, as presented in FIG. 7A-FIG. 7C, similarly indicated the presence (if any) and degree of invasive carcinoma present in a cancerous prostate tissue sample. For instance, ERG gene expression was low in the normal, non-cancerous prostate tissue sample (FIG. 7A), whereas ERG gene expression was high in the invasive carcinoma of the adenocarcinoma sample. The observations regarding FIG. 7A-FIG. 7C were further supported by IHC analysis as presented in FIG. 13A-FIG. 13F and FIG. 14A-FIG. 14F.

An alternative gene expression analysis sample preparation workflow can be performed on tissues that are fresh, frozen biopsy samples, if desired. These recommendations for protocol modifications are not required; however, it was demonstrated that when using fresh frozen tissue samples, gene expression analysis was sometimes enhanced comparative to that following the established protocol for using FFPE samples as defined in the cited literature.

For fresh frozen tissue samples, 10 μm cryostat sections are used on an array for gene expression spatial analysis. The cryosections are used immediately for downstream process steps or stored at −80° C. The arrayed tissue slides are warmed at 37° C. for 1 min and the tissues are fixed with formaldehyde in PBS for 10 min at room temperature and washed. As the tissues are not FFPE, there are no steps involving deparaffinization and decrosslinking, however it is contemplated that a decrosslinking step may be useful for fresh frozen tissues that are formaldehyde fixed. For the fresh frozen samples, following destaining if H&E is used, the arrays can be washed in TE buffer of pH 9 or SSC buffer.

Following destaining and washing, the protocol for fresh frozen tissues and FFPE as described in the cited literature is the same regardless of sample type.

### Example 2

### Gene Expression Analysis of Basal and Luminal Cells in Normal and Cancerous Prostate Tissues

Gene expression clustering analysis was used to characterize basal and luminal cells in normal, non-cancerous prostate tissue sample and in the adenocarcinoma prostate cancer tissue sample described above. As described below, the characterization revealed changes in basal and luminal cell co-localization in prostate cancer samples as compared to the normal, non-cancerous tissue sample.

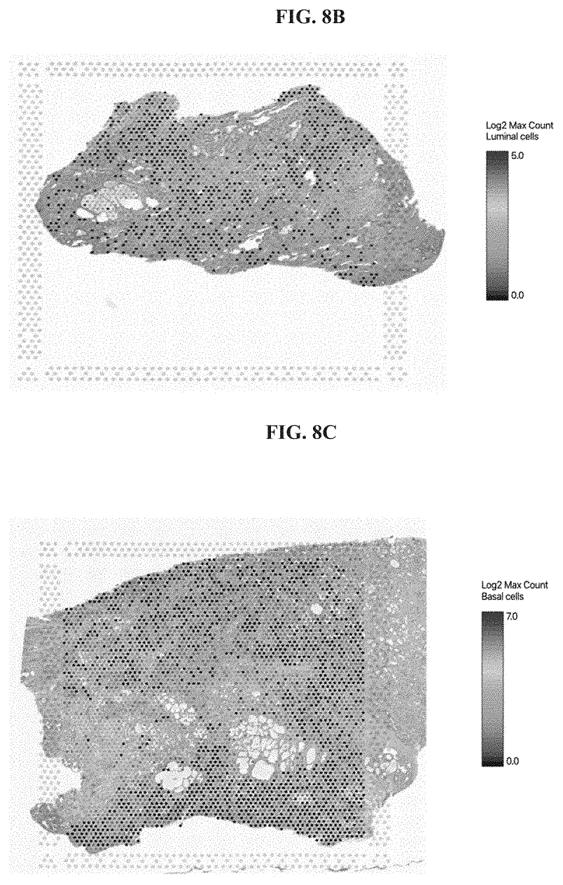

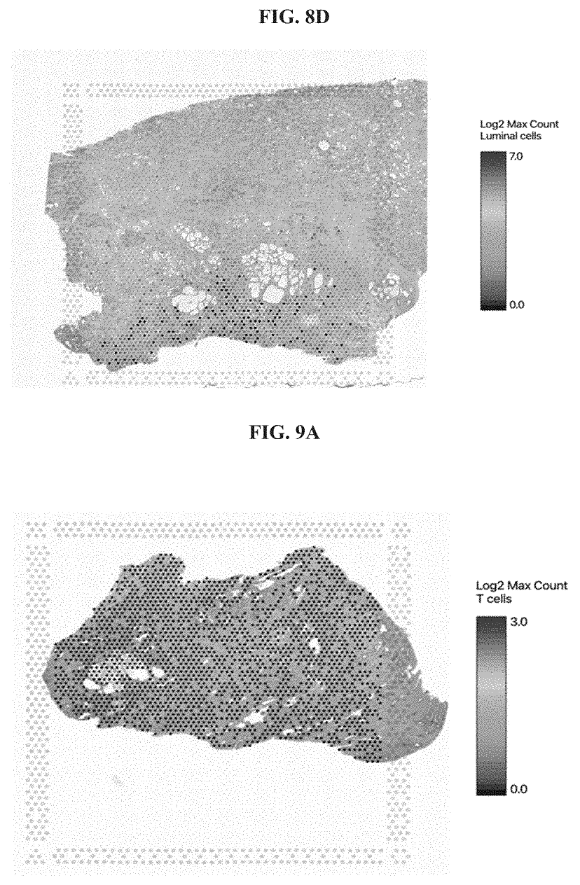

Gene expression of basal cell markers TP63, KRT5, and KRT14; and luminal cell markers CD24, KRT8, and KRT18, was analyzed in normal and adenocarcinoma prostate samples and presented in FIG. 8A-FIG. 8D. As shown in FIG. 8A and FIG. 8C, normal prostate tissue sample demonstrated a co-localization of basal and luminal cells throughout the tissue. Surprisingly, however, the adenocarcinoma prostate sample presented a high degree of disruption of basal and luminal cell co-localization, where the basal cells were mostly excluded from the invasive carcinoma region as opposed to the luminal cells, which were greatly expanded within and localized to the invasive carcinoma region. Interestingly, acinar cell carcinoma samples appears to show more luminal cell expression in Cluster 1 tumor region compared to Cluster 2.

### Example 3

### Gene Expression Analysis of Immune Cells in Normal and Cancerous Prostate Tissues

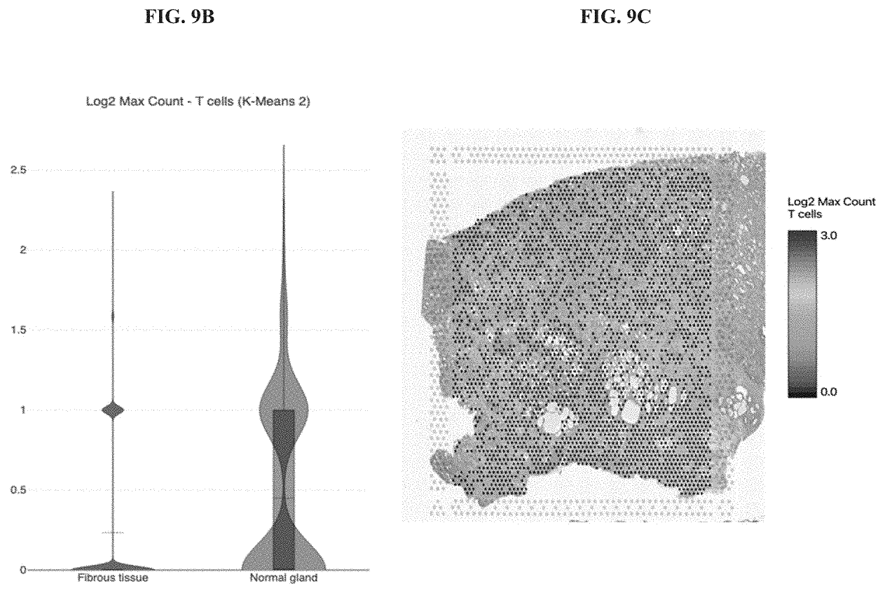

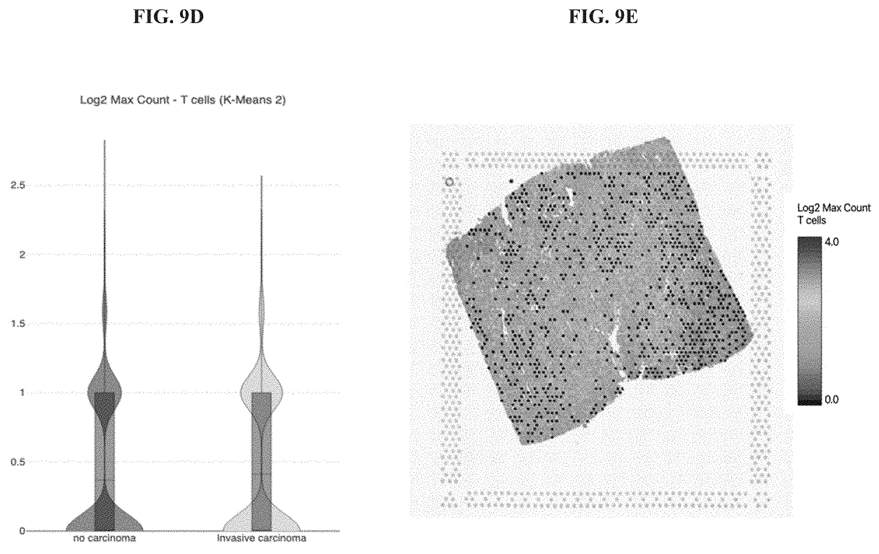

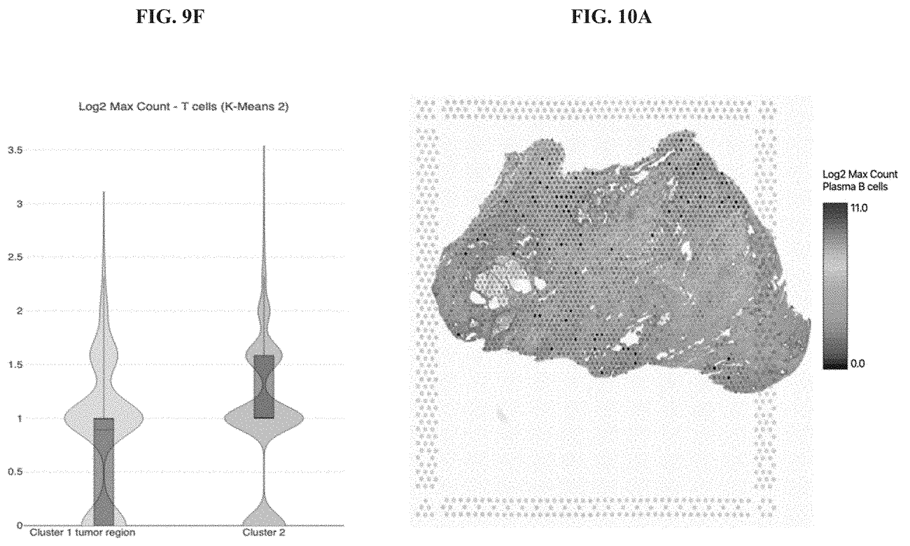

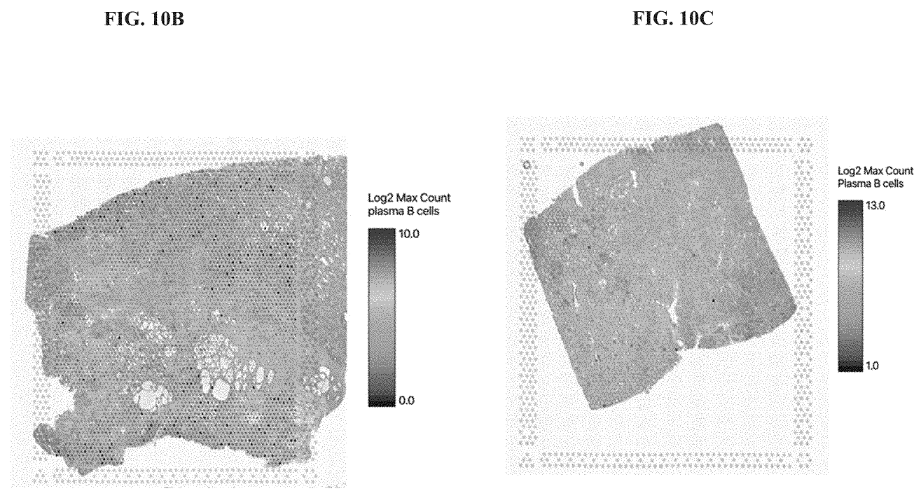

Gene expression analysis of immune cell markers was performed to characterize immune cell gene expression profiles and immune cell localization in a normal, non-cancerous prostate tissue sample, an adenocarcinoma prostate cancer tissue sample and an acinar prostate cancer tissue sample. B cells were found minimally in normal tissues, as presented in FIG. 11A, as compared to the adenocarcinoma prostate cancer tissue sample (FIG. 11B) and the acinar prostate cancer tissue sample (FIG. 10C). B cell markers CD79A and CD79B were upregulated and dispersed in higher abundance throughout the adenocarcinoma tissue sample (FIG. 11B) and the acinar prostate cancer tissue sample (FIG. 10C). Also, T cell markers CD3D, CD3E, CD8A and CD247 were upregulated and dispersed in higher abundance throughout the acinar prostate cancer tissue sample (FIG. 9E).

Surprisingly, markers of plasma B cells, IGHA1, IGHG1, JCHAIN, IGKC, IGLC1, were found up-regulated and in higher abundance around the border of the invasive carcinoma in the peritumoral region of the adenocarcinoma prostate cancer sample (FIG. 10B).

T-cell gene expression analysis was performed and revealed low level T-cell gene expression in the tissue sample and localization to the prostate glands, expression of T cell markers (CD3D, CD3E, CD8A and CD247) were highly upregulated and dispersed throughout the adenocarcinoma prostate sample, including throughout the invasive carcinoma region (FIG. 9A-FIG. 9F).

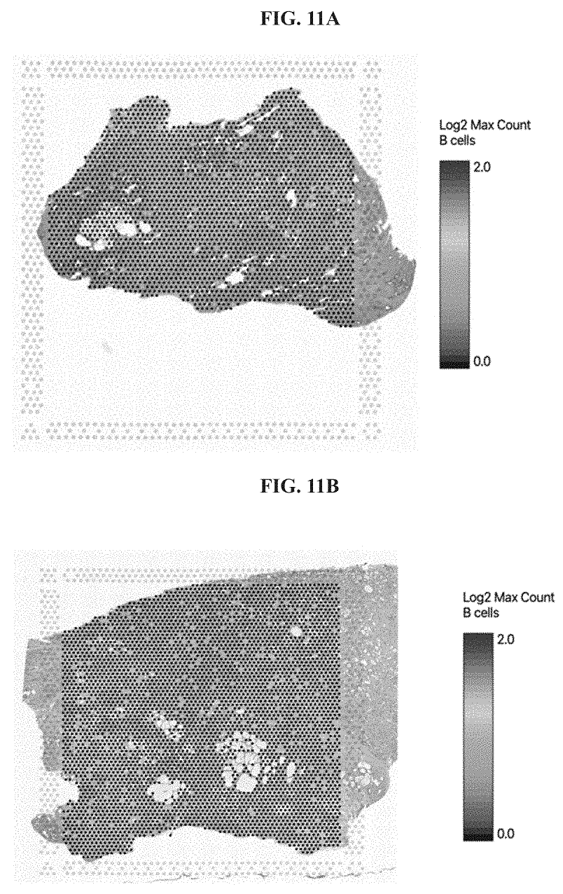

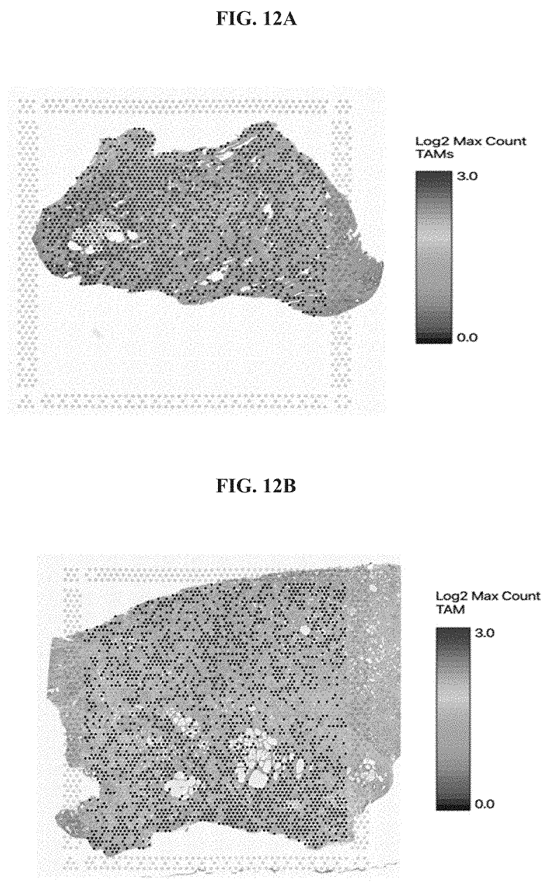

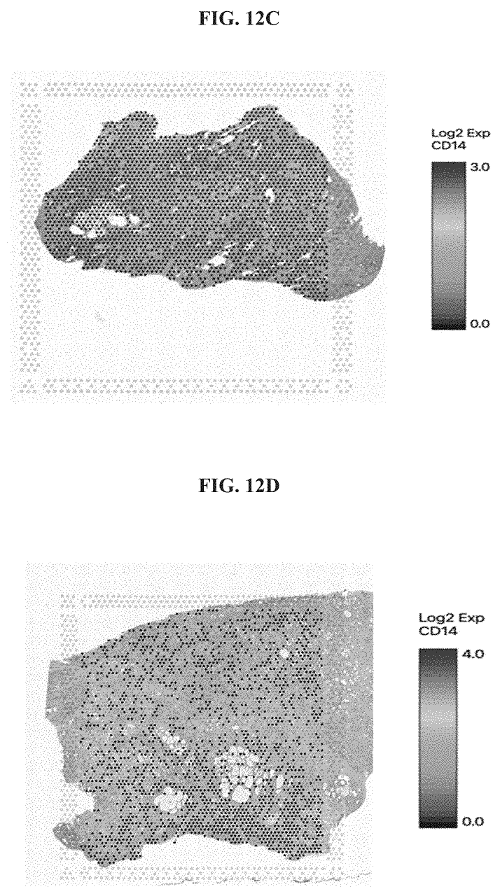

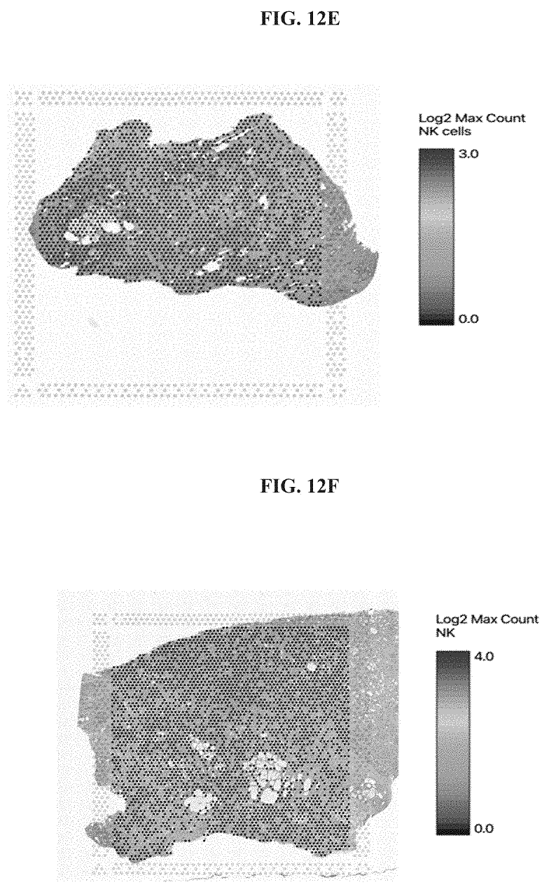

Further immune cell gene expression analysis was performed on the normal, non-cancerous tissue sample and the adenocarcinoma tissue sample, which further analysis is presented FIG. 12A-FIG. 12F. Tumor associated macrophages, or TAM cells, were characterized using gene makers CD163, MSR1 and MRC1, which were analyzed in normal (FIG. 12A) and adenocarcinoma (FIG. 12B) tissue samples. TAMs were observed in high numbers in the tumor microenvironment of solid tumors as demonstrated in FIG. 12B as marker genes for TAMs showed high upregulation throughout the adenocarcinoma tissue sample. A similar trend was observed for monocytes, as measured by gene markers CD14 (FIG. 12C and FIG. 12D), CD16, and FCGR3A (not shown), and natural killer (NK) cells (gene markers NKG7 and NCAM1) (FIG. 12E and FIG. 12F).

### Example 4

### Immunohistochemical Staining and Gene Expression Analysis for AMACR and ERG in Tissue Samples

In the present example, immunohistochemical staining was performed with monoclonal antibodies targeting AMACR and ERG, both known over expressed genes in prostate cancer, and the data was compared against the AMACR and ERG gene expression data from Example 1. Briefly, FFPE tissue sections were baked and dewaxed. The tissue sections were subsequently decrosslinked in a TE pH 9.0 buffer at 95° C. for 30 minutes and cooled to room temperature. Following decrosslinking, the tissues were blocked with a BSA/PBS solution.

Immunohistochemistry was carried out for 30 min at room temperature using either AMACR p504s rabbit monoclonal primary antibody (Roche SP116) or ERG EPR3864 anti-ERG rabbit monoclonal primary antibody, which were both obtained from Ventana (Roche Diagnostics). After incubation with the primary antibodies, immunohistochemistry was performed on the tissue samples using the Leica Novolink Polymer IHC kit per manufacturer's instructions. The tissues were H&E stained, mounted with Permount, and imaged on a Metafer Scanning Platform.

The IHC tissue images for AMACR and ERG were used for comparative analysis against the AMACR and ERG gene expression data from Example 1. As discussed above, AMACR has been implicated as an oncogene and indicator gene for prostate cancer.

As demonstrated by the results of Example 1, AMACR expression was highly upregulated in the invasive carcinoma region in the adenocarcinoma tissue, whereas the acinar cell carcinoma expression was not localized nor was it expressed as highly as that found in the adenocarcinoma tissue (FIG. 6A-FIG. 6C). IHC staining for AMACR was performed, and the images of the stained tissue samples are presented in FIG. 13A-FIG. 13F. As can be observed in FIG. 13A-FIG. 13F, whereas normal prostate tissue presented minimal to no AMACR protein based on the IHC staining (FIG. 13A and FIG. 13B), the adenocarcinoma (FIG. 13C and FIG. 13D) and acinar cell carcinoma (FIG. 13E and FIG. 13F) prostate tissue samples presented highly upregulated and overexpressed AMACR in invasive regions of the cancerous samples. Interestingly, AMACR was not highly upregulated and overexpressed throughout the whole tissue, which was contrary to the pathologist annotation of the acinar cell carcinoma as being wholly invasive.

Similar characteristics were observed when analyzing the samples for ERG levels. ERG, like AMACR, has been implicated as an oncogene, and furthermore ERG has been implicated as a nuclear marker found in prostate cancer. IHC staining for ERG was performed, and the images of the stained tissue samples are presented in FIG. 14A-FIG. 14F. As can be observed in FIG. 14A-FIG. 14F, whereas normal prostate tissue presented minimal to no ERG protein based on the IHC staining (FIG. 14A and FIG. 14B), the adenocarcinoma (FIG. 14C and FIG. 14D) and acinar cell carcinoma (FIG. 14E and FIG. 14F) prostate tissue samples presented highly upregulated and overexpressed ERG in the adenocarcinoma and acinar cell carcinoma prostate tissue samples. Interestingly, the adenocarcinoma shows upregulation of ERG in the invasive carcinoma regions, whereas there is upregulation in certain regions of the acinar carcinoma sample but not throughout the sample.

The invention is not to be limited in scope by the specific aspects described herein. Indeed, various modifications of the invention in addition to those described will become apparent to those skilled in the art from the foregoing description and accompanying figures. Such modifications are intended to fall within the scope of the appended claims.

All references (e.g., publications or patents or patent applications) cited herein are incorporated herein by reference in their entirety and for all purposes to the same extent as if each individual reference (e.g., publication or patent or patent application) was specifically and individually indicated to be incorporated by reference in its entirety for all purposes.

Other aspects are within the following claims.

What is claimed is:

1. A method for determining the presence or absence of prostate cancer in a subject, comprising:
(a) determining the expression level of two or more captured analytes or complements thereof from a biological sample from the subject, wherein the captured analytes or complements thereof were hybridized to capture probes on a spatial gene expression array, wherein the spatial gene expression array comprises a plurality of capture probes, a capture probe of the plurality comprising a spatial barcode and a capture domain, wherein the two or more captured analytes or complements thereof are from one of groups (i), (ii), (iii), or (iv) or a combination thereof:
(i) S100B, TGM4, SMOC1, LTF, ANPEP, SELE, PSCA, MT1G, ACTG2, WFDC2, SOCS3, PCP4, SYNM, GADD45B, TNC, GAS1, CNN1, ZNF185, DES, HSPB6, MSMB, MYLK, IGHG1, FLNC, ADAMTS4, MYL9, ADAM33, KRT5, PGM5, IGHV3-35, NEFH, JPH2, KRT15, TRIM29, SRD5A2, HSPB8, C11orf96, SPEG, ALDH2, FLNA, OLFM4, IGKV1D-12, TAGLN, TPM2, CRYAB, SMTN, KCNMB1, PMP22, ACTA2, and SYNPO2, or a fragment thereof;
(ii) ADGRF1, OR51C1P, KCNC2, PLA2G7, SPON2, CAMK2N1, KCNN2, OR51E2, AGTR1, GRIN3A, KHDRBS3, KCNH8, ATP8A2, TMEM26, LUZP2, HPN, PLA2G2A, COL2A1, UNC80, DNASE2B, HOXC6, SPOCK1, AKR7A3, LRRN1, GOLM1, GJB1, FGFRL1, GHR, ACSM3, MCCC2, TRGV9, ZNF385B, CYP39A1, SEC11C, H2AFJ, MYO6, GPR160, BMPR1B, MBOAT2, GALNT3, CPNE4, ACER3, PLEKHH1, C9orf152, and PAQR6, or a fragment thereof;
(iii)OR51E1, FGFR3, SPON2, KCNG3, OR51C1P, ARG2, DEGS1, OR51E2, TRGJP2, CPNE4, GOLM1, RDH11, ACSM1, PLEKHH1, SLC30A4, TRGC1, GDF11, KHDRBS3, SPOCK1, GDF15, FGFRL1, TRGV9, LRRN1, PLA2G7, TMSB15A, KCNN2, MARC1, PTPRT, C5orf30, COLEC12, HPN, MYO6, ADGRF1, UBE2E3, HGD, NSD2, KIF21A, HIST1H2AC, LETM1, COL9A2, BEND4, GJB1, ARFGEF3, PRAC1, F3, and SAMD5, or a fragment thereof;
(iv)IGHA1, JCHAIN, CD79A, IGHJ6, IGKC, SAA2, CCL19, IGLC1, SAA1, IGHG2, POU2AF1, DERL3, S100A9, IGHG1, LTB, C3, MZB1, IGKV4-1, RARRES1, LTF, IGHG3, IGHJ2, IGLV3-1, LYZ, LSP1, OLFM4, FCGBP, CFB, PDZK1IP1, KRT7, CORO1A, IL7R, PRDM1, CYBA, IGHM, LCN2, SERPINF1, HCLS1, CD53, PTPRC, PTGDS, NNMT, IGFBP4, WFDC2, CP, ITGB2, C1R, CLU, KRT15, and FHL2, or a fragment thereof; and
(b) identifying the presence or absence of prostate cancer in the subject based on the determined expression level of the two or more analytes or complements thereof.

2. The method of claim 1, wherein the captured analytes or complements thereof are obtained by:
(a) providing two or more sets of target analyte probes, wherein a first probe of the set comprises a capture probe binding domain and a second probe of the set comprises a functional sequence, and wherein the first and second probes hybridize adjacent to each other on the target analyte from the biological sample,
(b) ligating the first and second probe, thereby generated a ligation product, and
(c) releasing the ligation product from the target analyte,
thereby allowing the capture probe binding domain of the ligation product to hybridize to the capture domain of the capture probe on the spatial gene expression array.

3. The method of claim 2, wherein the determining the expression level of two or more analytes comprises:
(a) extending the capture probe and the ligation product, thereby generating an extension product that includes the spatial barcode and target analyte sequence, or complements thereof,
(b) releasing the extension product,
(c) amplifying the released extension product to generate a plurality of nucleic acids comprising the spatial barcode and target analyte sequences, or complements thereof, and
(d) sequencing the amplified nucleic acids, thereby determining the expression level of the two or more analytes in the biological sample.

4. The method of claim 3, wherein sequencing comprises sequence by synthesis, sequence by hybridization, sequence by ligation, or nanopore sequencing.

5. The method of claim 1, wherein the determining the expression level of two or more analytes comprises:
(a) determining the expression level of two or more analytes associated with a luminal cell in one or more locations in the biological sample, wherein the one or more analytes is CD24 or a fragment thereof, KRT8 or a fragment thereof, and KRT18 or a fragment thereof, or combinations thereof; and
(b) correlating the presence or absence of prostate cancer in the subject based on the determined expression level of the analytes associated with the luminal cells.

6. The method of claim 1, wherein the determining the expression level of two or more analytes comprises:
(a) determining the expression level of two or more analytes associated with an immune cell in one or more locations in the biological sample, wherein the two or more analytes is CD3D or a fragment thereof, CD3E or a fragment thereof, CD4 or a fragment thereof, CD8A or a fragment thereof, CD247 or a fragment thereof; CD79A or a fragment thereof, CD79B or a fragment thereof, IGHA1 or a fragment thereof, IGHG1 or a fragment thereof, JCHAIN or a fragment thereof, IGKC or a fragment thereof, IGLC1 or a fragment thereof, or combinations thereof, in the biological sample from the subject; and
(b) correlating the presence or absence of prostate cancer in the subject based on the determined expression level of the analytes associated with the immune cells.

7. The method of claim 1, wherein the biological sample was disposed on the spatial gene expression array or on a substrate without the spatial gene expression array.

8. The method of claim 7, wherein the biological sample that was disposed on the substrate without the spatial gene expression array was aligned with a spatial gene expression array, such that at least a portion of the biological sample was aligned with at least a portion of the spatial gene expression array.

9. A method of monitoring a treatment regimen for prostate cancer in a subject, comprising:
(a) determining the gene expression levels of two or more captured analytes from the biological sample from one of groups (i), (ii), (iii), or (iv) or a combination thereof, wherein the expression levels of the two or more analytes had been previously determined for the subject prior to being treated for prostate cancer:
(i) S100B, TGM4, SMOC1, LTF, ANPEP, SELE, PSCA, MT1G, ACTG2, WFDC2, SOCS3, PCP4, SYNM, GADD45B, TNC, GAS1, CNN1, ZNF185, DES, HSPB6, MSMB, MYLK, IGHG1, FLNC, ADAMTS4, MYL9, ADAM33, KRT5, PGM5, IGHV3-35, NEFH, JPH2, KRT15, TRIM29, SRD5A2, HSPB8, C11orf96, SPEG, ALDH2, FLNA, OLFM4, IGKV1D-12, TAGLN, TPM2, CRYAB, SMTN, KCNMB1, PMP22, ACTA2, and SYNPO2, or a fragment thereof;
(ii) ADGRF1, OR51C1P, KCNC2, PLA2G7, SPON2, CAMK2N1, NPY, KCNN2, OR51E2, AGTR1, GRIN3A, KHDRBS3, KCNH8, ATP8A2, TMEM26, LUZP2, HPN, PLA2G2A, COL2A1, UNC80, DNASE2B, HOXC6, SPOCK1, AKR7A3, LRRN1, GOLM1, GJB1, FGFRL1, GHR, ACSM3, MCCC2, TRGV9, ZNF385B, CYP39A1, SEC11C, H2AFJ, MYO6, GPR160, BMPR1B, MBOAT2, GALNT3, CPNE4, ACER3, PLEKHH1, C9orf152, and PAQR6, or a fragment thereof;
(iii) OR51E1, FGFR3, SPON2, KCNG3, OR51C1P, ARG2, DEGS1, OR51E2, TRGJP2, CPNE4, GOLM1, RDH11, ACSM1, PLEKHH1, SLC30A4, TRGC1, GDF11, KHDRBS3, SPOCK1, GDF15, FGFRL1, TRGV9, LRRN1, PLA2G7, TMSB15A, KCNN2, MARC1, PTPRT, C5orf30, COLEC12, HPN, MYO6, ADGRF1, UBE2E3, HGD, NSD2, KIF21A, HIST1H2AC, LETM1, COL9A2, BEND4, GJB1, ARFGEF3, PRAC1, F3, and SAMD5, or a fragment thereof;
(iv) IGHA1, JCHAIN, CD79A, IGHJ6, IGKC, SAA2, CCL19, IGLC1, SAA1, IGHG2, POU2AF1, DERL3, S100A9, IGHG1, LTB, C3, MZB1, IGKV4-1, RARRES1, LTF, IGHG3, IGHJ2, IGLV3-1, LYZ, LSP1, OLFM4, FCGBP, CFB, PDZK1IP1, KRT7, CORO1A, IL7R, PRDM1, CYBA, IGHM, LCN2, SERPINF1, HCLS1, CD53, PTPRC, PTGDS, NNMT, IGFBP4, WFDC2, CP, ITGB2, C1R, CLU, KRT15, and FHL2, or a fragment thereof;
(b) comparing the determined gene expression levels of the two or more analytes before treatment of the subject with the determined gene expression levels of the two or more analytes during treatment of the subject for prostate cancer and based on that comparison, perform one or more of;
(i) administering an additional treatment to the subject based on the comparative difference in the expression levels of the two or more analytes;
(ii) adjusting the treatment based on the comparative difference in the expression levels of the two or more analytes; or
(iii) ceasing the treatment based on the comparative difference in the expression levels of the two or more analytes.

10. The method of claim 9, wherein the determining gene expression levels for comparison comprises:
(a) determining the gene expression levels of two or more analytes associated with a luminal cell in one or more locations in the biological sample, wherein the two or more analytes is CD24 or a fragment thereof, KRT8 or a fragment thereof, and KRT18 or a fragment thereof, or combinations thereof, in the biological sample from the subject; and/or
(b) determining the gene expression levels of two or more analytes associated with a basal cell in one or more locations in the biological sample, wherein the two or more analytes is TP63, KRT5, KRT14, or combinations thereof, in the biological sample from the subject.

11. The method of claim 9, wherein the determining gene expression levels for comparison comprises determining the gene expression levels of two or more analytes associated with an immune cell in two or more locations in the biological sample, wherein the two or more analytes is CD3D or a fragment thereof, CD3E or a fragment thereof, CD4 or a fragment thereof, CD8A or a fragment thereof, CD247 or a fragment thereof; CD79A or a fragment thereof, CD79B or a fragment thereof, IGHAl or a fragment thereof, IGHG1 or a fragment thereof, JCHAIN or a fragment thereof, IGKC or a fragment thereof, IGLC1 or a fragment thereof.

12. The method of claim 9, wherein the determining comprises:
(a) providing two or more sets of target analyte probes to the biological sample, wherein a first probe of the set comprises a capture probe binding domain and a second probe of the set comprises a functional sequence, and wherein the first and second probes hybridize adjacent to each other on the target analyte,
(b) ligating the first and second probe, thereby generated a ligation product,
(c) releasing the ligation product from the target analyte,
(d) permeabilizing the biological sample,
(e) hybridizing the ligation product to the capture probe on the spatial gene expression array,
extending the capture probe and the ligation product, thereby generating an extension product that includes the spatial barcode and target analyte sequence, or complements thereof,
(g) releasing the extension product,
(h) amplifying the released extension product to generate a plurality of nucleic acids comprising the spatial barcode and target analyte sequences, or complements thereof, and
sequencing the amplified nucleic acids, thereby determining the expression levels of the two or more analytes in the biological sample prior to and after the subject is treated for prostate cancer.

13. A method of identifying the presence or absence of invasive prostate cancer in a subject, comprising:
(a) determining the gene expression levels of two or more captured analytes or complements thereof associated with a luminal cell in a biological sample from the subject, wherein the two or more captured analytes or complements thereof associated with a luminal cell were hybridized to capture probes on a spatial gene expression array, wherein the spatial gene expression array comprises a plurality of capture probes, a capture probe of the plurality comprising a spatial barcode and a capture domain, wherein the two or more captured analytes associated with a luminal cell comprise CD24 or a fragment thereof, KRT8 or a fragment thereof, and KRT18 or a fragment thereof, or combinations thereof;
(b) determining the gene expression levels of two or more captured analytes or complements thereof associated with a basal cell in one or more locations in the biological sample, wherein the two or more captured analytes or complements thereof associated with a basal cell were hybridized to capture probes on the spatial gene expression array, wherein the two or more captured analytes associated with a basal cell comprise TP63, KRT5, KRT14, or combinations thereof; and
(c) identifying the presence or absence of invasive prostate cancer in the subject based on the determined gene expression levels of the two or more captured analytes or complements thereof associated with a luminal cell and the two or more captured analytes or complements thereof associated with a basal cell.

14. The method of claim 13, wherein the captured analytes or complements thereof are obtained by:
(a) providing two or more sets of target analyte probes, wherein a first probe of the set comprises a capture probe binding domain and a second probe of the set comprises a functional sequence, and wherein the first and second probes hybridize adjacent to each other on the target analyte from the biological sample,
(b) ligating the first and second probe, thereby generated a ligation product, and
(c) releasing the ligation product from the target analyte, thereby allowing the capture probe binding domain of the ligation product to hybridize to the capture domain of the capture probe on the spatial gene expression array.

15. The method of claim 14, wherein the determining the expression level of two or more analytes comprises:
(a) extending the capture probe and the ligation product, thereby generating an extension product that includes the spatial barcode and target analyte sequence, or complements thereof,
(b) releasing the extension product,
(c) amplifying the released extension product to generate a plurality of nucleic acids comprising the spatial barcode and target analyte sequences, or complements thereof, and
(d) sequencing the amplified nucleic acids, thereby determining the expression level of the two or more analytes in the biological sample.

16. The method according to claim 1, wherein the determining the gene expression levels of two or more analytes comprises assaying serially obtained biological samples from the subject at a plurality of time points and determining the expression levels of the two or more analytes in the serially obtained biological samples from the subject.

17. The method according to claim 1, wherein the determining the gene expression levels of the two or more analytes further comprises comparing the gene expression levels of the two or more analytes with the gene expression levels of the two or more analytes from a reference tissue sample.

18. The method according to claim 1, wherein the prostate cancer is adenocarcinoma, acinar cell carcinoma, ductal adenocarcinoma, transitional cell (or urothelial) cancer, squamous cell cancer, or small cell prostate cancer.

19. The method according to claim 1, further comprising imaging the biological sample.

20. The method of claim 19, wherein the imaging comprises one or more stains comprising hematoxylin and eosin.

21. The method of claim 19, wherein the imaging comprises one or more stains comprising one or more optical labels, wherein the one or more optical labels are selected from the group consisting of: fluorescent, radioactive, chemiluminescent, colorimetric labels, and combination thereof.

22. The method according to claim 1, wherein the biological sample is a tissue section or biopsy, and wherein the tissue section is a formalin fixed paraffin embedded tissue sample, a frozen tissue sample, or a fresh tissue sample.

23. The method of claim 22, wherein the biological sample comprises serial tissue sections or serial biopsies.

24. The method according to claim 1, wherein the two or more analytes are protein analytes, or mRNA analytes, or a combination thereof.

25. The method according to claim 1, wherein the expression level is a modulated expression level, an elevated expression level, or a decreased expression level compared to a reference expression level.

26. A system comprising:
(a) a storage element operable to store a dataset of a plurality of biological samples, wherein the dataset comprises, for each biological sample: analyte data for a plurality of analytes captured at a plurality of spatial locations from a biological sample; image data of the biological sample; and registration data of the imaged data, and wherein at least one biological sample is a reference sample and a second biological sample is a prostate tissue sample suspected of being cancerous;
(b) a computer program capable of linking the analyte data according to the spatial locations determined from a biological sample; and
(c) a processor operable to process the dataset through a machine learning module to determine the presence or absence of prostate cancer in the biological sample.

27. The system of claim 26, wherein the plurality of analytes comprise two or more analytes from one or more of (i), (ii), (iii), or (iv):
(i) S100B, TGM4, SMOC1, LTF, ANPEP, SELE, PSCA, MT1G, ACTG2, WFDC2, SOCS3, PCP4, SYNM, GADD45B, TNC, GAS1, CNN1, ZNF185, DES, HSPB6, MSMB, MYLK, IGHG1, FLNC, ADAMTS4, MYL9, ADAM33, KRT5, PGM5, IGHV3-35, NEFH, JPH2, KRT15, TRIM29, SRD5A2, HSPB8, C11orf96, SPEG, ALDH2, FLNA, OLFM4, IGKV1D-12, TAGLN, TPM2, CRYAB, SMTN, KCNMB1, PMP22, ACTA2, and SYNPO2, or a fragment thereof;
(ii) ADGRF1, OR51C1P, KCNC2, PLA2G7, SPON2, CAMK2N1, KCNN2, OR51E2, AGTR1, GRIN3A, KHDRBS3, KCNH8, ATP8A2, TMEM26, LUZP2, HPN, PLA2G2A, COL2A1, UNC80, DNASE2B, HOXC6, SPOCK1, AKR7A3, LRRN1, GOLM1, GJB1, FGFRL1, GHR, ACSM3, MCCC2, TRGV9, ZNF385B, CYP39A1, SEC11C, H2AFJ, MYO6, GPR160, BMPR1B, MBOAT2, GALNT3, CPNE4, ACER3, PLEKHH1, C9orf152, and PAQR6, or a fragment thereof;
(iii) OR51E1, FGFR3, SPON2, KCNG3, OR51C1P, ARG2, DEGS1, OR51E2, TRGJP2, CPNE4, GOLM1, RDH11, ACSM1, PLEKHH1, SLC30A4, TRGC1, GDF11, KHDRBS3, SPOCK1, GDF15, FGFRL1, TRGV9, LRRN1, PLA2G7, TMSB15A, KCNN2, MARC1, PTPRT, C5orf30, COLEC12, HPN, MYO6, ADGRF1, UBE2E3, HGD, NSD2, KIF21A, HIST1H2AC, LETM1, COL9A2, BEND4, GJB1, ARFGEF3, PRAC1, F3, and SAMD5, or a fragment thereof; or
(iv) IGHA1, JCHAIN, CD79A, IGHJ6, IGKC, SAA2, CCL19, IGLC1, SAA1, IGHG2, POU2AF1, DERL3, S100A9, IGHG1, LTB, C3, MZB1, IGKV4-1, RARRES1, LTF, IGHG3, IGHJ2, IGLV3-1, LYZ, LSP1, OLFM4, FCGBP, CFB, PDZK1IP1, KRT7, CORO1A, IL7R, PRDM1, CYBA, IGHM, LCN2, SERPINF1, HCLS1, CD53, PTPRC, PTGDS, NNMT, IGFBP4, WFDC2, CP, ITGB2, C1R, CLU, KRT15, and FHL2, or a fragment thereof.

28. The system of claim 27, wherein the plurality of analytes comprises at least one of the following sets of analytes:
a) two or more of CD24, KRT8, KRT18, or combinations thereof,
b) two or more of CD3D, CD3E, CD4, CD8A, CD247; CD79A, CD79B, IGHA1, IGHG1, JCHAIN, IGKC, IGLC1, fragments thereof, or combinations thereof,
c) two or more of TP63, KRT5, KRT14, fragments thereof, or combinations thereof,
d) two or more of CD4, Foxp3, IL17RB, CTLA4, FANK1, HAVCR1, CD25, GITR, LAG-3, CD127, fragments thereof, or combinations thereof,
e) two or more of CD4, CD3D, S100A4, IL7R, IFNG, fragments thereof, or combinations thereof,
f) two or more of CD4, IL7R, ICOS, CTLA4, TNFRSF4, TNFRS18, fragments thereof, or combinations thereof,
g) two or more of CD4, CD3D, IL17A, GZMA, S100A4, fragments thereof, or combinations thereof, and
h) two or more of CD8, CD3D, S100A4, IFNG, GZMB, GZMA, IL2RB, fragments thereof, or combinations thereof.
